(19) United States
(12) Patent Application Publication
(10) Pub. No.: US 2026/0083728 A1
(43) Pub. Date: Mar. 26, 2026

(54) COMPOUNDS FOR TREATING INFECTIONS WITH PARASITIC PROTOZOA

(71) Applicants: OHIO STATE INNOVATIION FOUNDATION, Columbus, OH (US); UNIVERSITY OF KANSAS, Lawrence,, KS (US)

(72) Inventors: Karl WERBOVETZ, Worthington, OH (US); Pankaj SHARMA, Sayreville, NJ (US); Chris LA ROSA, Columbus, OH (US); Zhuo WANG, Lawrence, KS (US); Yiru JIN, Lawrence, KS (US); Mei FENG, Lawrence, KS (US)

(21) Appl. No.: 19/111,246

(22) PCT Filed: Sep. 18, 2023
(86) PCT No.: PCT/US2023/033029

§ 371 Date: Mar. 12, 2025

Related U.S. Application Data
(60) Provisional application No. 63/407,387, filed on Sep. 16, 2022.

(51) Int. Cl. A61K 31/496; A61P 33/02; C07D 207/325; C07D 213/36; C07D 231/12; C07D 233/61; C07D 249/08; C07D 295/192

(52) U.S. Cl. CPC A61K 31/496; A61P 33/02; C07D 207/325; C07D 213/36; C07D 231/12; C07D 233/61; C07D 249/08; C07D 295/192

(57) ABSTRACT

This disclosure describes compounds of Formula I and Formula II, pharmaceutically acceptable salts or derivatives thereof, and pharmaceutical compositions thereof, useful in treating infections caused by parasitic protozoa, in particular protozoa of the genera Leishmania or Trypanosoma.

### CROSS-REFERENCE TO RELATED APPLICATIONS

This application claims the benefit of priority to U.S. Provisional Patent Application No. 63/407,387 filed Sep. 16, 2022, the disclosure of which is incorporated herein by reference in its entirety.

### STATEMENT REGARDING FEDERALLY SPONSORED RESEARCH OR DEVELOPMENT

This invention was made with government support under grant/contract number AI139198 awarded by the National Institutes of Health. The government has certain rights in the invention.

### TECHNICAL FIELD

This disclosure relates to compounds for treating medical disorders, and more particularly to compounds useful in treating infections with parasitic protozoa.

### BACKGROUND

Approximately 1 million new cases of leishmaniasis occur each year; the visceral form of this disease is almost always fatal if not treated. It is widely reported that visceral leishmaniasis ranks second to malaria as the leading cause of death due to a parasitic disease (see R. Wamai, J. Kahn, J. McGloin, G. Ziaggi, Visceral leishmaniasis: a global overview, J. Glob. Health Sci., 2 (2020) e3; and S. DebRoy, O. Prosper, A. Mishoe, A. Mubayi, Challenges in modeling complexity of neglected tropical diseases: a review of dynamics of visceral leishmaniasis in resource limited settings, Emerg. Themes Epidemiol., 14 (2017) 10). Despite the severity of this problem, the therapeutic armamentarium available against leishmaniasis has significant weaknesses, and new drug candidates are needed to fill the antileishmanial drug discovery pipeline. Sterol metabolism has been considered as a prime target for antileishmanial agents. One of the most effective current antileishmanial drugs, amphotericin B, exerts its antiparasitic (and antifungal) effects by binding to ergosterol, the primary sterol in Leishmania and fungi, and thus disrupts cellular processes in these pathogens. Inhibitors of 14α-demethylase (CYP51), an enzyme involved in the biosynthesis of ergosterol, are currently used as antifungal drugs. CYP51 inhibitors such as ketoconazole, fluconazole, and posaconazole and have been considered as potential antileishmanial drugs, albeit with variable clinical results (see J. Rashid, K. Wasunna, G. Gachihi, P. Nyakundi, J. Mbugua, G. Kirigi, The efficacy and safety of ketoconazole in visceral leishmaniasis, East Afr. Med. J., 71 (1994) 392-395; S. Sundar, V. Singh, N. Agrawal, D. Gibbs, H. Murray, Treatment of kala-azar with oral fluconazole, Lancet, 348 (1996) 614; and A. Paniz Mondolfi, C. Stavropoulos, T. Gelanew, E. Loucas, A. Perez Alvarez, G. Benaim, B. Polsky, G. Schoenian, E. Sordillo, Successful treatment of Old World cutaneous leishmaniasis caused by Leishmania infantum with posaconazole, Antimicrob. Agents Chemother., 55 (2011) 1774-1776).

In 2011, Verma et al. reported a novel protein in Leishmania donovani, CYP5122A1, with an undefined role in sterol metabolism (see S. Verma, A. Mehta, C. Shaha, CYP5122A1, a novel cytochrome P450 is essential for survival of Leishmania donovani, PLoS One, 6 (2011) e25273). The Zhuo (Michael) Wang group at the University of Kansas later expressed and purified this protein and have been working to establish its biological function, while the Kai Zhang group at Texas Tech University has demonstrated the essentiality of CYP5122A1 in both the mammalian stage and vector stage of Leishmania donovani (see M. Wang, Y. Jin, S. Basu, M. Feng, Y. Ning, I. Munasinghe, A. Joachim, J. Li, R. Madden, H. Burks, P. Gao, C. Perera, K. Werbovetz, K. Zhang, CYP5122A1 encodes an essential sterol C4-methyl oxidase in Leishmania donovani and determines the antileishmanial activity of antifungal azoles. Res. Sq., (2023) doi: 10.21203/rs.3.rs-3185204/v1, preprint). Hybrid compounds prepared in the Werbovetz lab incorporating the features of both antifungal azoles and the arylimidamides, a potent class of in vitro antileishmanial compounds including DB766 and DB2002 that show moderate in vivo antileishmanial efficacy (see M. Wang, X. Zhu, A. Srivastava, Q. Liu, J. Sweat, T. Pandharkar, C. Stephens, E. Riccio, T. Parman, M. Munde, S. Mandal, R. Madhubala, R. Tidwell, W. Wilson, D. Boykin, J. Hall, D. Kyle, K. Werbovetz, Novel arylimidamides for the treatment of visceral leishmaniasis, Antimicrob. Agents Chemother., 54 (2010) 2507-2516; and X. Zhu, A. Farahat, M. Mattamana, A. Joice, T. Pandharkar, E. Holt, M. Banerjee, J. Gragg, L. Hu, A. Kumar, S. Yang, M. Wang, D. Boykin, K. Werbovetz, Synthesis and pharmacological evaluation of mono-arylimidamides as antileishmanial agents Bioorg. Med. Chem. Lett., 26 (2016) 2551-2556), exhibit good in vitro antileishmanial activity, but the most potent compound in this series, AA3-33, displays modest in vivo efficacy (see A. Abdelhameed, M. Feng, A. C. Joice, E. M. Zywot, Y. Jin, C. La Rosa, X. Liao, H. L. Meeds, Y. Kim, J. Li, C. A. McElroy, M. Z. Wang, K. A. Werbovetz, Synthesis and antileishmanial evaluation of arylimidamide-azole hybrids containing a phenoxyalkyl linker, ACS Infect. Dis., 7 (2021) 1901-1922). The activity of the antileishmanial hybrid compounds against L. donovani CYP51 and L. donovani CYP5122A1 is in the high nanomolar range based on the 7-benzyloxy-4-trifluoromethylcoumarin (BFC) fluorogenic inhibition assay developed by the Wang group.

There is a clear need for additional therapeutics that are useful in treating infections with parasitic protozoa such as leishmaniasis.

### SUMMARY

The present disclosure provides compounds and compositions which are useful in treating infections caused by parasitic protozoa, for example, protozoa of the genera Leishmania and Trypanosoma. Methods of making and using said compositions are also provided.

In one aspect, a compound is provided of Formula I

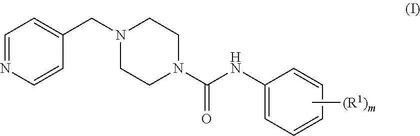

or a pharmaceutically acceptable salt or derivative thereof;
wherein all variables are as defined herein.

In another aspect, a compound is provided of Formula II

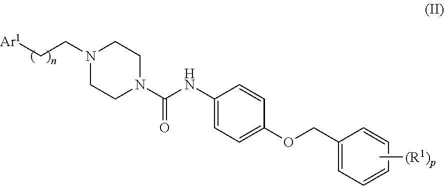

or a pharmaceutically acceptable salt thereof;
wherein all variables are as defined herein.

A pharmaceutical composition is also provided comprising a compound described herein, or a pharmaceutically acceptable salt or derivative thereof, and a pharmaceutically acceptable carrier or excipient.

A method of treating or preventing an infection with a parasitic protozoa in a subject in need thereof comprising administering to the subject a therapeutically effective amount of a compound described herein, or a pharmaceutically acceptable salt or derivative thereof, or a pharmaceutical composition thereof.

In some aspects, the parasitic protozoa expresses CYP51 and/or CYP5122A1.

In some aspects, the parasitic protozoa comprises a Leishmania parasite (e.g., which causes cutaneous leishmaniasis, mucocutaneous leishmaniasis, or visceral leishmaniasis) or a Trypanosoma parasite (e.g., which causes African trypanosomiasis, Chagas disease, nagana, and surra).

The details of one or more aspects of the disclosure are set forth in the description below. Other features, objects, and advantages of the disclosure will be apparent from the description and the claims.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual aspects described and illustrated herein has discrete components and features which may be readily separated from or combined with the features of any of the other several aspects without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a compound”, “an infection”, or “a parasite”, includes, but is not limited to, two or more such compounds, compositions, or parasites, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “therapeutically effective amount” refers to an amount that is sufficient to achieve the desired therapeutic result or to have an effect on undesired symptoms but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. It is generally preferred that a maximum dose of the pharmacological agents of the disclosure (alone or in combination with other therapeutic agents) be used, that is, the highest safe dose according to sound medical judgment. It will be understood by those of ordinary skill in the art however, that a patient may insist upon a lower dose or tolerable dose for medical reasons, psychological reasons or for virtually any other reasons.

A response to a therapeutically effective dose of a disclosed compound or composition can be measured by determining the physiological effects of the treatment or medication, such as the decrease or lack of disease symptoms following administration of the treatment or pharmacological agent. Other assays will be known to one of ordinary skill in the art and can be employed for measuring the level of the response. The amount of a treatment may be varied for example by increasing or decreasing the amount of a disclosed compound and/or pharmaceutical composition, by changing the disclosed compound and/or pharmaceutical composition administered, by changing the route of administration, by changing the dosage timing and so on. Dosage can vary, and can be administered in one or more dose administrations daily, for one or several days. Guidance can be found in the literature for appropriate dosages for given classes of pharmaceutical products.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the terms “optional” or “optionally” means that the subsequently described event or circumstance can or cannot occur, and that the description includes instances where said event or circumstance occurs and instances where it does not.

As used interchangeably herein, “subject,” “individual,” or “patient” can refer to a vertebrate organism, such as a mammal (e.g., human). “Subject” can also refer to a cell, a population of cells, a tissue, an organ, or an organism, preferably to human and constituents thereof.

As used herein, the terms “treating” and “treatment” can refer generally to obtaining a desired pharmacological and/or physiological effect. The effect can be, but does not necessarily have to be, prophylactic in terms of preventing or partially preventing a disease, symptom or condition thereof, such as an infection. The effect can be therapeutic in terms of a partial or complete cure of a disease, condition, symptom or adverse effect attributed to the disease, disorder, or condition. The term “treatment” as used herein can include any treatment of a disorder in a subject, particularly a human and can include any one or more of the following. (a) preventing the disease from occurring in a subject which may be predisposed to the disease but has not yet been diagnosed as having it; (b) inhibiting the disease, i.e., arresting its development; and (c) relieving the disease, i.e., mitigating or ameliorating the disease and/or its symptoms or conditions. The term “treatment” as used herein can refer to both therapeutic treatment alone, prophylactic treatment alone, or both therapeutic and prophylactic treatment. Those in need of treatment (subjects in need thereof) can include those already with the disorder and/or those in which the disorder is to be prevented. As used herein, the term “treating”, can include inhibiting the disease, disorder or condition, e.g., impeding its progress; and relieving the disease, disorder, or condition, e.g., causing regression of the disease, disorder and/or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

As used herein, “dose,” “unit dose,” or “dosage” can refer to physically discrete units suitable for use in a subject, each unit containing a predetermined quantity of a disclosed compound and/or a pharmaceutical composition thereof calculated to produce the desired response or responses in association with its administration.

As used herein, “therapeutic” can refer to treating, healing, and/or ameliorating a disease, disorder, condition, or side effect, or to decreasing in the rate of advancement of a disease, disorder, condition, or side effect.

### Chemical Definitions

Compounds are described using standard nomenclature. Unless defined otherwise, all technical and scientific terms used herein have the same meaning as is commonly understood by one of skill in the art to which this disclosure belongs.

The compounds described herein include enantiomers, mixtures of enantiomers, diastereomers, tautomers, racemates and other isomers, such as rotamers, as if each is specifically described, unless otherwise indicated or otherwise excluded by context. It is to be understood that the compounds provided herein may contain chiral centers. Such chiral centers may be of either the (R-) or (S-) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R-) form is equivalent, for compounds that undergo epimerization in vivo, to administration of the compound in its (S-) form. Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

A dash (“-”) that is not between two letters or symbols is used to indicate a point of attachment for a substituent. For example, —(C═O)NH₂ is attached through the carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable. For example, when the substituent is oxo (i.e., ═O) then two hydrogens on the atom are replaced. For example, a pyridyl group substituted by oxo is a pyridine. Combinations of substituents and/or variables are permissible only if such combinations result in stable compounds or useful synthetic intermediates. A stable active compound refers to a compound that can be isolated and can be formulated into a dosage form with a shelf life of at least one month. A stable manufacturing intermediate or precursor to an active compound is stable if it does not degrade within the period needed for reaction or other use. A stable moiety or substituent group is one that does not degrade, react or fall apart within the period necessary for use. Non-limiting examples of unstable moieties are those that combine heteroatoms in an unstable arrangement, as typically known and identifiable to those of skill in the art.

Any suitable group may be present on a “substituted” or “optionally substituted” position that forms a stable molecule and meets the desired purpose of the disclosure and includes, but is not limited to: alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, orthiol.

The terms for various functional groups as used herein are not intended to be limited to monovalent radicals and may include polyvalent radical groups as appropriate, such as divalent, trivalent, tetravalent, pentavalent, and hexavalent groups, and the like, based on the position and location of such groups in the compounds described herein as would be readily understood by the skilled person.

“Alkyl” is a straight chain or branched saturated aliphatic hydrocarbon group. In certain aspects, the alkyl is C₁-C₂, C₁-C₃, or C₁-C₆ (i.e., the alkyl chain can be 1, 2, 3, 4, 5, or 6 carbons in length). The specified ranges as used herein indicate an alkyl group with length of each member of the range described as an independent species. For example, C₁-C₆alkyl as used herein indicates an alkyl group having from 1, 2, 3, 4, 5, or 6 carbon atoms and is intended to mean that each of these is described as an independent species and C₁-C₄alkyl as used herein indicates an alkyl group having from 1, 2, 3, or 4 carbon atoms and is intended to mean that each of these is described as an independent species. When C₀-Cₙalkyl is used herein in conjunction with another group, for example (C₃-C₇cycloalkyl)C₀-C₄alkyl, or —C₀-C₄(C₃-C₇cycloalkyl), the indicated group, in this case cycloalkyl, is either directly bound by a single covalent bond (C₀alkyl), or attached by an alkyl chain, in this case 1, 2, 3, or 4 carbon atoms. Alkyls can also be attached via other groups such as heteroatoms, as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In one aspect, the alkyl group is optionally substituted as described herein.

“Cycloalkyl” is a saturated mono- or multi-cyclic hydrocarbon ring system. When composed of two or more rings, the rings may be joined together in a fused or bridged fashion. Non-limiting examples of typical cycloalkyl groups include cyclopropyl, cyclobutyl, cyclopentyl, cyclohexyl, and cycloheptyl. In one aspect, the cycloalkyl group is optionally substituted as described herein.

“Alkenyl” is a straight or branched chain aliphatic hydrocarbon group having one or more carbon-carbon double bonds, each of which is independently either cis or trans, that may occur at a stable point along the chain. Non-limiting examples include C₂-C₄alkenyl and C₂-C₆alkenyl (i.e., having 2, 3, 4, 5, or 6 carbons). The specified ranges as used herein indicate an alkenyl group having each member of the range described as an independent species, as described above for the alkyl moiety. Examples of alkenyl include, but are not limited to, ethenyl and propenyl. In one aspect, the alkenyl group is optionally substituted as described herein.

“Alkynyl” is a straight or branched chain aliphatic hydrocarbon group having one or more carbon-carbon triple bonds that may occur at any stable point along the chain, for example, C₂-C₄alkynyl or C₂-C₆alkynyl (i.e., having 2, 3, 4, 5, or 6 carbons). The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, and 5-hexynyl. In one aspect, the alkynyl group is optionally substituted as described herein.

“Alkoxy” is an alkyl group as defined above covalently bound through an oxygen bridge (—O—). Examples of alkoxy include, but are not limited to, methoxy, ethoxy, n-propoxy, isopropoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, and 3-methylpentoxy. Similarly, an “alkylthio” or “thioalkyl” group is an alkyl group as defined above with the indicated number of carbon atoms covalently bound through a sulfur bridge (—S—). In one aspect, the alkoxy group is optionally substituted as described herein.

“Alkanoyl” is an alkyl group as defined above covalently bound through a carbonyl (C═O) bridge. The carbonyl carbon is included in the number of carbons, for example C₂alkanoyl is a CH₃(C═O)— group. In one aspect, the alkanoyl group is optionally substituted as described herein.

“Halo” or “halogen” indicates, independently, any of fluoro, chloro, bromo or iodo.

“Aryl” indicates an aromatic group containing only carbon in the aromatic ring or rings. In one aspect, the aryl group contains 1 to 3 separate or fused rings and is 6 to 14 or 18 ring atoms, without heteroatoms as ring members. When indicated, such aryl groups may be further substituted with carbon or non-carbon atoms or groups. Such substitution may include fusion to a 4- to 7- or 5- to 7-membered saturated or partially unsaturated cyclic group that optionally contains 1, 2, or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In one aspect, aryl groups are pendant. An example of a pendant ring is a phenyl group substituted with a phenyl group. In one aspect, the aryl group is optionally substituted as described herein.

The term “heterocycle” refers to saturated and partially saturated heteroatom-containing ring radicals, where the heteroatoms may be selected from N, O, and S. The term heterocycle includes monocyclic 3-12 members rings, as well as bicyclic 5-16 membered ring systems (which can include fused, bridged, or spiro bicyclic ring systems). It does not include rings containing —O—O—, —O—S—, and —S—S— portions. Examples of saturated heterocycle groups including saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4- to 6-membered monocyclic groups containing 1 to 2 oxygen atoms and 1 to 3 nitrogen atoms [e.g., morpholinyl]; and saturated 3- to 6-membered heteromonocyclic groups containing 1 to 2 sulfur atoms and 1 to 3 nitrogen atoms [e.g., thiazolidinyl]. Examples of partially saturated heterocycle radicals include, but are not limited, dihydrothienyl, dihydropyranyl, dihydrofuryl, and dihydrothiazolyl. Examples of partially saturated and saturated heterocycle groups include, but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3,-dihydro-1H-benzo[d]isothazol-6-yl, dihydropyranyl, dihydrofuryl, and dihydrothiazolyl. Bicyclic heterocycle includes groups wherein the heterocyclic radical is fused with an aryl radical wherein the point of attachment is the heterocycle ring. Bicyclic heterocycle also includes heterocyclic radicals that are fused with a carbocyclic radical. Representative examples include, but are not limited to, partially unsaturated condensed heterocyclic groups containing 1 to 5 nitrogen atoms, for example indoline and isoindoline, partially unsaturated condensed heterocyclic groups containing 1 to 2 oxygen atoms and 1 to 3 nitrogen atoms, partially unsaturated condensed heterocyclic groups containing 1 to 2 sulfur atoms and 1 to 3 nitrogen atoms, and saturated condensed heterocyclic groups containing 1 to 2 oxygen or sulfur atoms.

“Heteroaryl” refers to a stable monocyclic, bicyclic, or multicyclic aromatic ring which contains from 1 to 4, or in some aspects 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 4, or in some aspects from 1 to 3 or from 1 to 2, heteroatoms selected from N, O, S, B, or P, with remaining ring atoms being carbon. In one aspect, the only heteroatom is nitrogen. In one aspect, the only heteroatom is oxygen. In one aspect, the only heteroatom is sulfur. Monocyclic heteroaryl groups typically have from 5 to 6 ring atoms. In some aspects, bicyclic heteroaryl groups are 8- to 10-membered heteroaryl groups, that is groups containing 8 or 10 ring atoms in which one 5-, 6-, or 7-membered aromatic ring which contains from 1 to 4 heteroatoms selected from N, O, S, B, or P is fused to a second aromatic or non-aromatic ring, wherein the point of attachment is an aromatic ring. When the total number of S and O atoms in the heteroaryl ring exceeds 1, these heteroatoms are not adjacent to one another within the ring. In one aspect, the total number of S and O atoms in the heteroaryl ring is not more than 2. In another aspect, the total number of S and O atoms in the heteroaryl ring is not more than 1. Examples of heteroaryl groups include, but are not limited to, pyridinyl, imidazolyl, imidazopyridinyl, pyrimidinyl, pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, triazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl.

A “pharmaceutically acceptable salt” is a derivative of the disclosed compound in which the parent compound is modified by making inorganic and organic, pharmaceutically acceptable, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent compound formed, for example, from inorganic or organic salts. Example of such salts include, but are not limited to, those derived from inorganic acids such as hydrochloric, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using a different acid that produced the same counterion. Lists of additional suitable salts may be found, e.g., in Remington's Pharmaceutical Sciences, 17^{th} ed., Mack Publishing Company, Easton, PA., p. 1418 (1985).

As used herein, the term “derivative” refers to a compound having a structure derived from the structure of a parent compound (e.g., a compound disclosed herein) and whose structure is sufficiently similar to those disclosed herein and based upon that similarity would be expected by one skilled in the art to exhibit the same or similar activities and utilities as the claimed compounds, or to induce, as a precursor, the same or similar activities and utilities as the claimed compound. Exemplary derivatives include, but are not limited to, salts, esters, amides, salts of esters or amides, and N-oxides of a parent compound.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers

### Compounds

The present disclosure provides compounds which are useful in treating infections caused by parasitic protozoa, for example, parasite of the genera Leishmania and Trypanosoma.

In one aspect, a compound is provided of Formula I

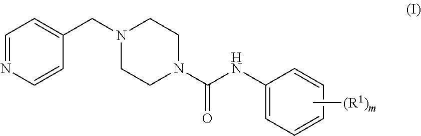

or a pharmaceutically acceptable salt or derivative thereof;
wherein:
R¹ is selected from halo, cyano, azido, C₁-C₆ alkyl, C₁-C₆ haloalkyl, C₂-C₆ alkenyl, C₂-C₆ alkynyl, (C₃-C₆ cycloalkyl)-(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{x}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;
m is 0, 1, 2, 3, 4, or 5;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y} each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol;
with the proviso that

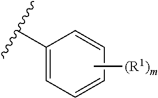

cannot be 3,5-dimethylphenyl.

In some aspects of Formula I, m is 0. In some aspects of Formula I, m is 1. In some aspects of Formula I, m is 2. In some aspects of Formula I, m is 3. In some aspects of Formula I, m is 4. In some aspects of Formula I, m is 5.

In some aspects of Formula I, R¹ is independently selected at each occurrence from halo, C₁-C₆ alkyl, C₁-C₆ alkoxy, C₁-C₆ haloalkyl, and C₁-C₆ haloalkoxy. In some aspects of Formula I, R¹ is independently selected at each occurrence from methyl, ethyl, propyl, isopropyl, butyl, tert-butyl, methoxy, ethoxy, propoxy, isopropoxy, tert-butoxy, fluoro, chloro, bromo, trifluoromethyl, and trifluoromethoxy.

In some aspects of Formula I,

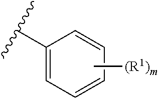

is selected from:

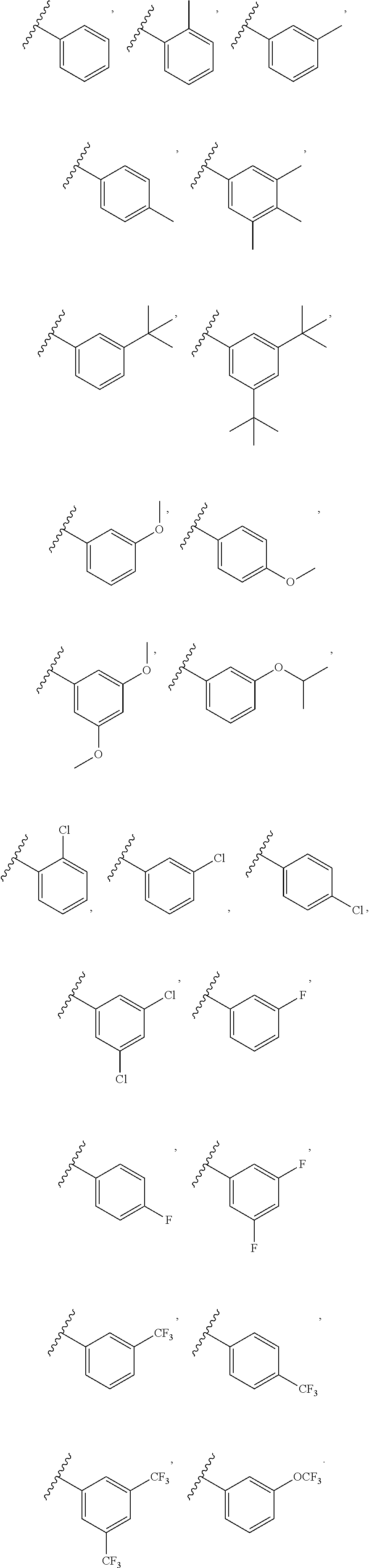

In some aspects of Formula I,
m is 0, 1, 2, 3, 4, or 5; and

R¹ is independently selected at each occurrence from halo, C₁-C₆ alkyl, C₁-C₆ alkoxy, C₁-C₆ haloalkyl, and C₁-C₆ haloalkoxy.

In some aspects of Formula I,
m is 0, 1, 2, 3, 4, or 5; and
R¹ is independently selected at each occurrence from methyl, ethyl, propyl, isopropyl, butyl, tert-butyl, methoxy, ethoxy, propoxy, isopropoxy, tert-butoxy, fluoro, chloro, bromo, trifluoromethyl, and trifluoromethoxy.

In some aspects, a compound is provided selected from:

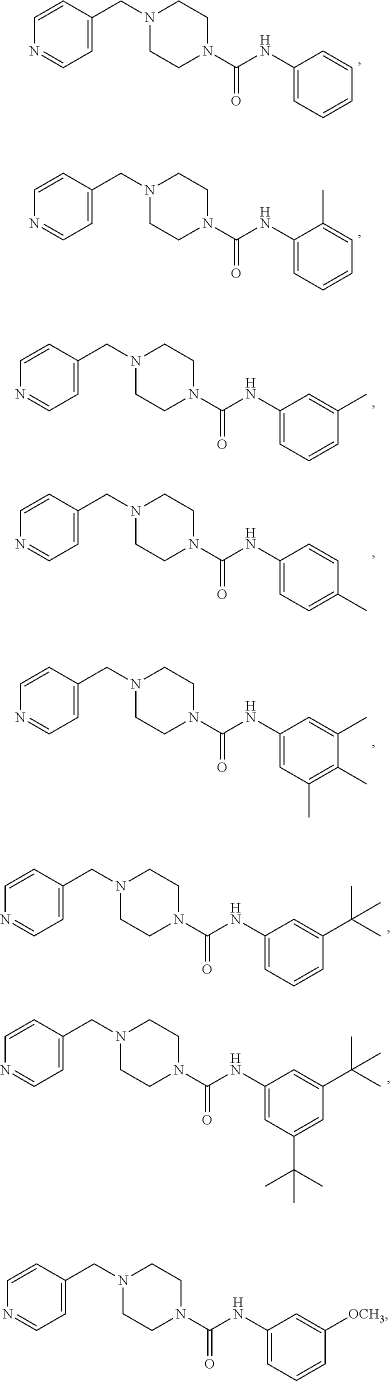

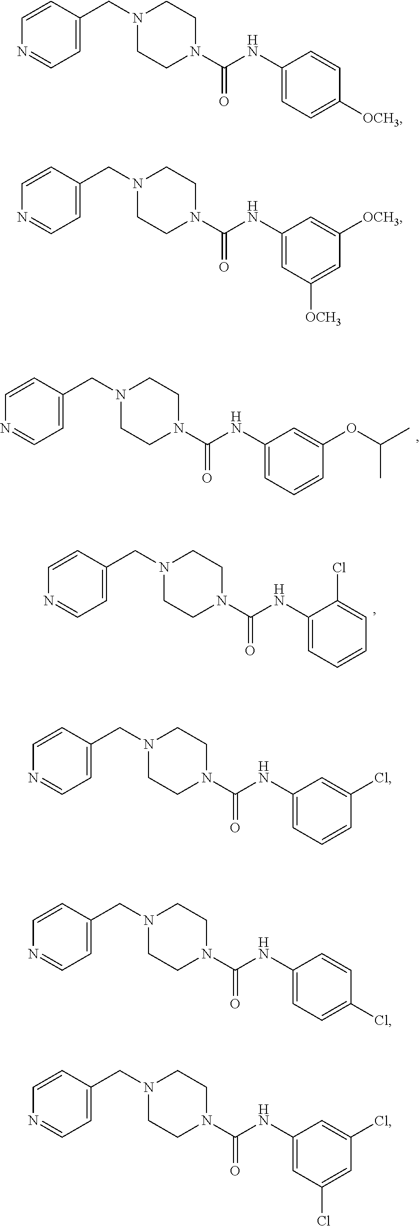

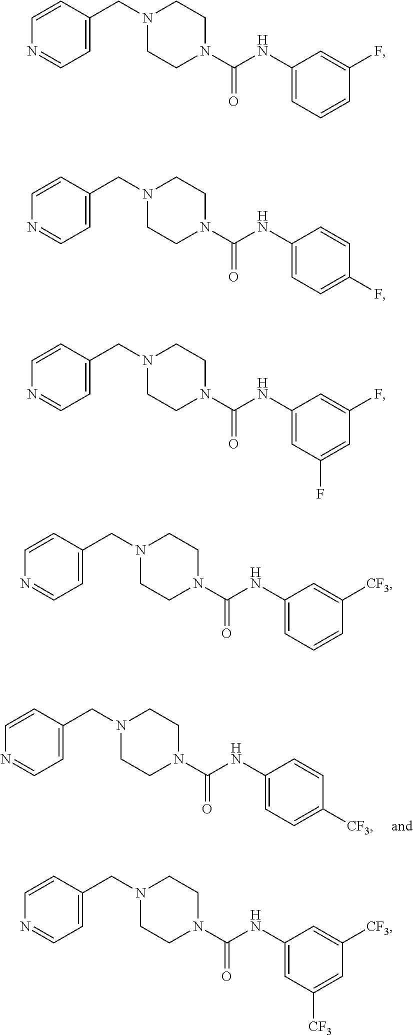 [SMILES: O=C(Nc1cccc(F)c1)N1CCN(Cc2ccncc2)CC1 ; O=C(Nc1ccc(F)cc1)N1CCN(Cc2ccncc2)CC1 ; O=C(Nc1cc(F)cc(F)c1)N1CCN(Cc2ccncc2)CC1 ; O=C(Nc1cccc(C(F)(F)F)c1)N1CCN(Cc2ccncc2)CC1 ; O=C(Nc1ccc(C(F)(F)F)cc1)N1CCN(Cc2ccncc2)CC1 ; O=C(Nc1cc(C(F)(F)F)cc(C(F)(F)F)c1)N1CCN(Cc2ccncc2)CC1]

or a pharmaceutically acceptable salt or derivative thereof.

In another aspect, a compound is provided of Formula II

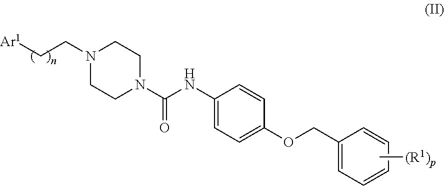

or a pharmaceutically acceptable salt thereof;
wherein:
R¹ is selected from halo, cyano, azido, C₁-C₆ alkyl, C₁-C₆ haloalkyl, C₂-C₆ alkenyl, C₂-C₆ alkynyl, (C₃-C₆ cycloalkyl)-(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{x}S(O)—(C₀-C₃ alkyl)-, and R^{z}S(O)₂—(C₀-C₃ alkyl)-;
n is 0, 1, or 2;
or, in an alternative aspect, n is 3;
p is 1, 2, 3, 4, or 5;
Ar¹ is 6- to 10-membered monocyclic or bicyclic aryl or 5- to 10-membered monocyclic or bicyclic heteroaryl, wherein Ar¹ is optionally substituted with one or more Z groups as allowed by valency;
Z is selected from hydrogen, halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{x}S(O)₂—O—(C₀-C₃ alkyl)-, R^{x}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;
R^{x} and R^{y} are independently selected at each occurrence from hydrogen, C₁-C₆alkyl, C₁-C₆haloalkyl, C₂—C(alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

In some aspects of Formula II, Ar¹ is phenyl optionally substituted with one or more (for example 1, 2, 3, 4, or 5) Z groups. In some aspects of Formula II, Ar¹ is 5- to 6-membered monocyclic heteroaryl optionally substituted with one or more (for example, 1, 2, 3, or 4) Z groups.

In some aspects of Formula II, Ar¹ is selected from:

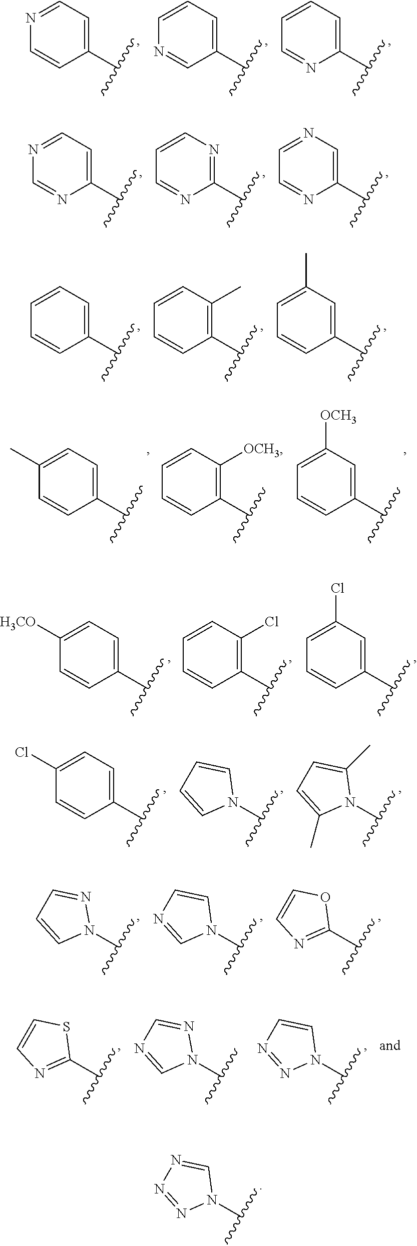

In some aspects of Formula II, n is 0. In some aspects of Formula II, n is 1. In some aspects of Formula II, n is 2. In some alternative aspects of Formula II, n is 3.

In some aspects of Formula II, p is 1. In some aspects of Formula II, p is 2. In some aspects of Formula II, p is 3. In some aspects of Formula II, p is 4. In some aspects of Formula II, p is 5.

In some aspects of Formula II, R¹ is independently selected at each occurrence from halo, C₁-C₆ alkyl, C₁-C₆ alkoxy, C₁-C₆ haloalkyl, and C₁-C₆ haloalkoxy. In some aspects of Formula II, R¹ is independently selected at each occurrence from methyl, ethyl, propyl, isopropyl, butyl, tert-butyl, methoxy, ethoxy, propoxy, isopropoxy, tert-butoxy, fluoro, chloro, bromo, trifluoromethyl, and trifluoromethoxy.

In some aspects of Formula II,

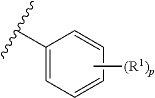

is selected from:

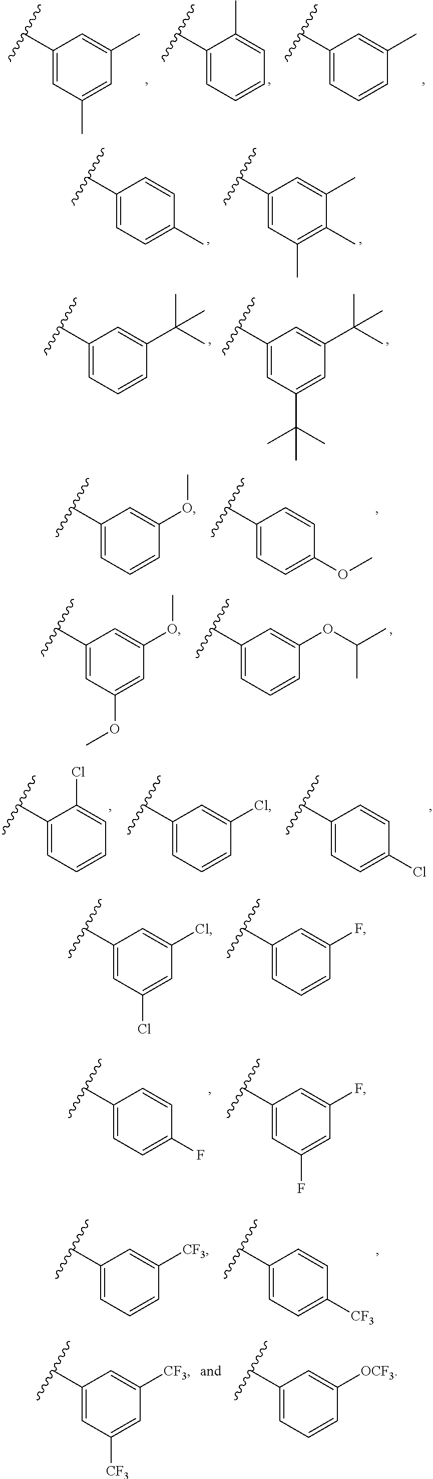

In some aspects of Formula II,
Ar¹ is phenyl optionally substituted with 1, 2, 3, 4, or 5 Z groups;
n is 0, 1, or 2;
p is 1, 2, 3, 4, or 5; and
R¹ is independently selected at each occurrence from halo, C₁-C₆ alkyl, C₁-C₆ alkoxy, C₁-C₆ haloalkyl, and C₁-C₆ haloalkoxy.

In some aspects of Formula II,
Ar¹ is phenyl optionally substituted with 1, 2, 3, 4, or 5 Z groups;
n is 0, 1, or 2;
p is 1, 2, 3, 4, or 5; and
R¹ is independently selected at each occurrence from methyl, ethyl, propyl, isopropyl, butyl, tert-butyl, methoxy, ethoxy, propoxy, isopropoxy, tert-butoxy, fluoro, chloro, bromo, trifluoromethyl, and trifluoromethoxy.

In some aspects of Formula II,
Ar¹ is 5- to 6-membered monocyclic heteroaryl optionally substituted with 1, 2, 3, or 4 Z groups;
n is 0, 1, or 2;
p is 1, 2, 3, 4, or 5; and
R¹ is independently selected at each occurrence from halo, C₁-C₆ alkyl, C₁-C₆ alkoxy, C₁-C₆ haloalkyl, and C₁-C₆ haloalkoxy.

In some aspects of Formula II,
Ar¹ is 5- to 6-membered monocyclic heteroaryl optionally substituted with 1, 2, 3, or 4 Z groups;
n is 0, 1, or 2;
p is 1, 2, 3, 4, or 5; and
R¹ is independently selected at each occurrence from methyl, ethyl, propyl, isopropyl, butyl, tert-butyl, methoxy, ethoxy, propoxy, isopropoxy, tert-butoxy, fluoro, chloro, bromo, trifluoromethyl, and trifluoromethoxy.

In some aspects of Formula II,
Ar¹ is selected from

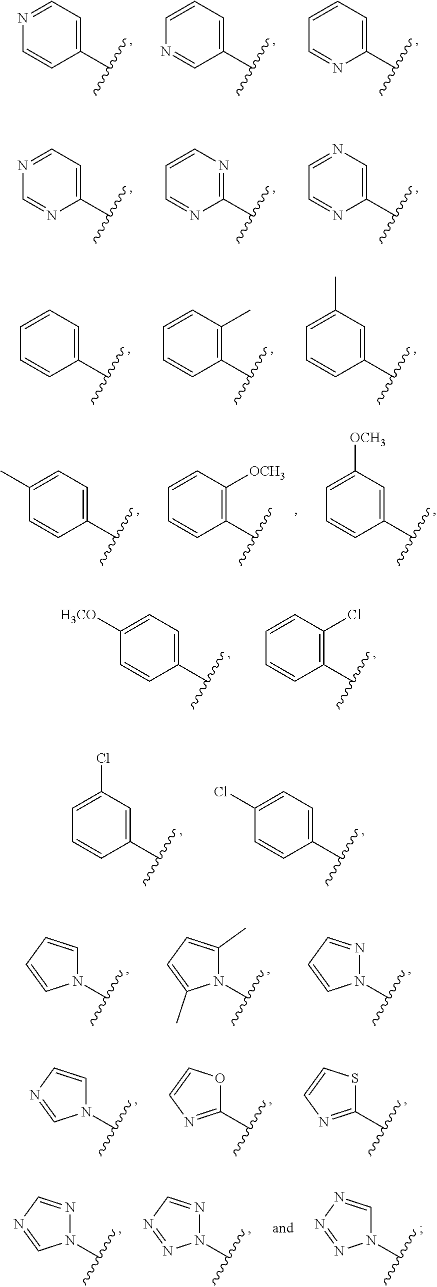

n is 0, 1, or 2;
p is 1, 2, 3, 4, or 5; and
R¹ is independently selected at each occurrence from halo, C₁-C₆ alkyl, C₁-C₆ alkoxy, C₁-C₆ haloalkyl, and C₁-C₆ haloalkoxy.

In some aspects of Formula II,
Ar¹ is selected from

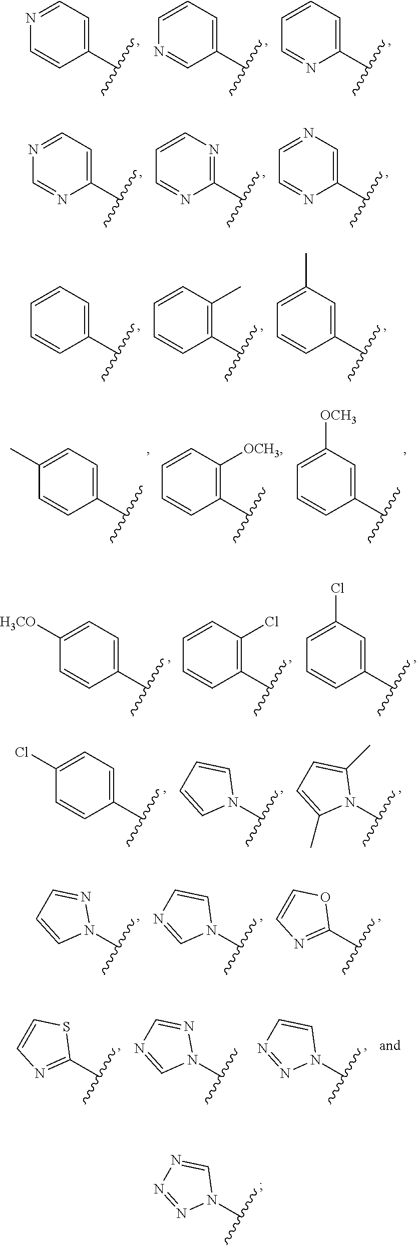

n is 0, 1, or 2;
p is 1, 2, 3, 4, or 5; and
R¹ is independently selected at each occurrence from methyl, ethyl, propyl, isopropyl, butyl, tert-butyl, methoxy, ethoxy, propoxy, isopropoxy, tert-butoxy, fluoro, chloro, bromo, trifluoromethyl, and trifluoromethoxy.

In some aspects, a compound is provided selected from.

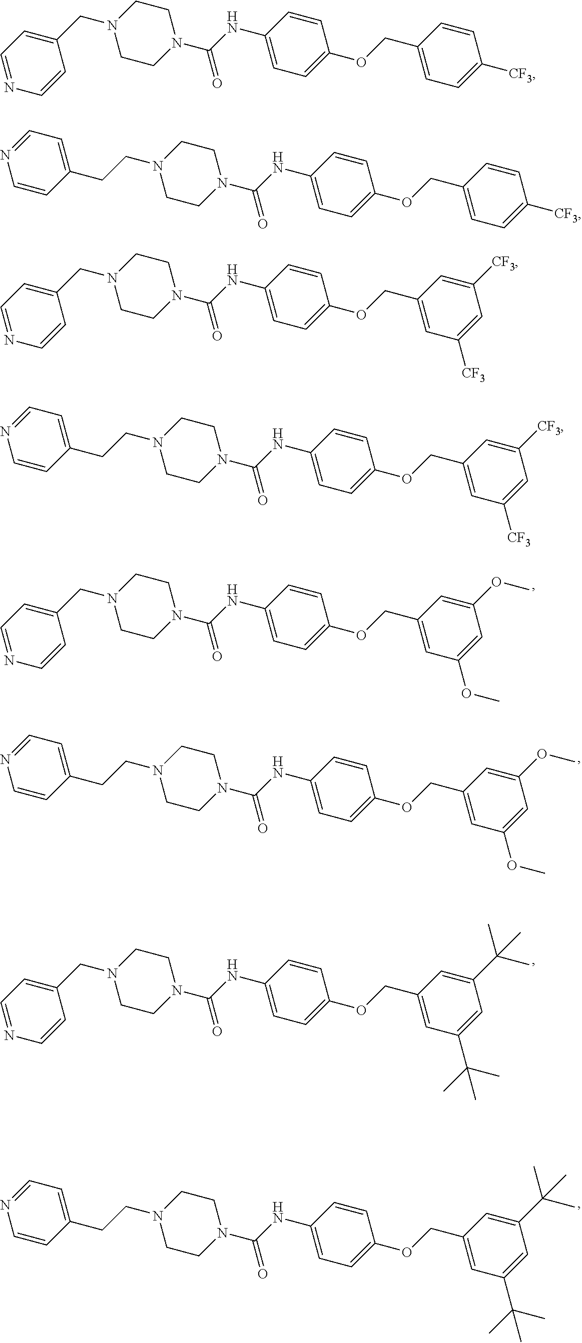

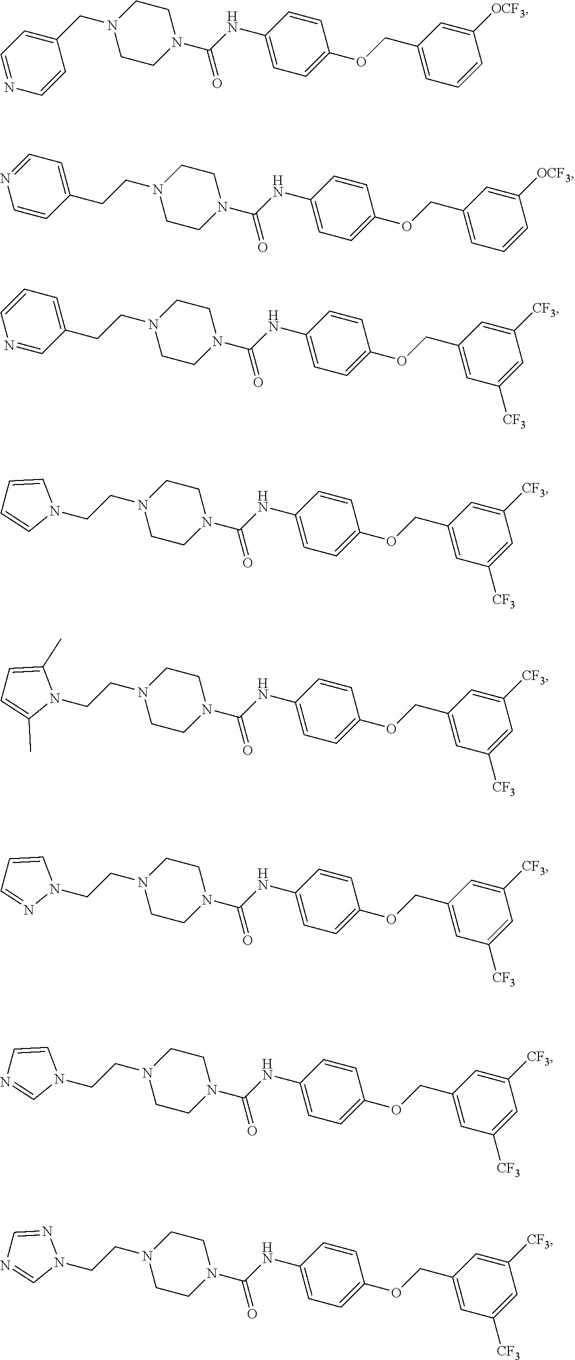

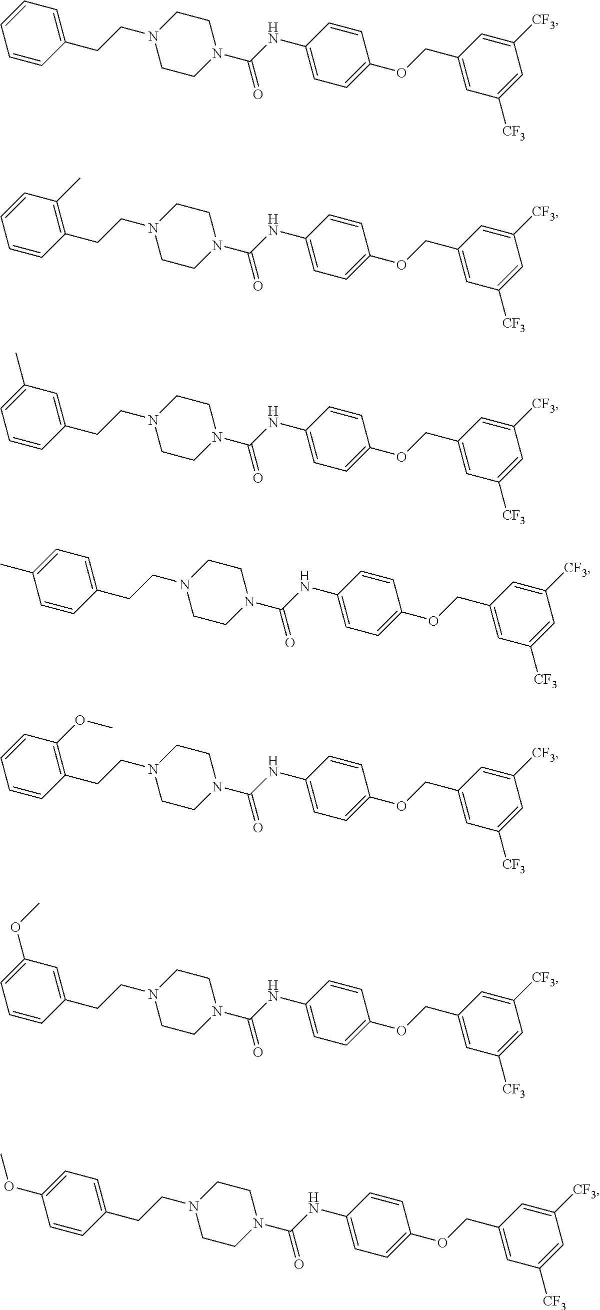

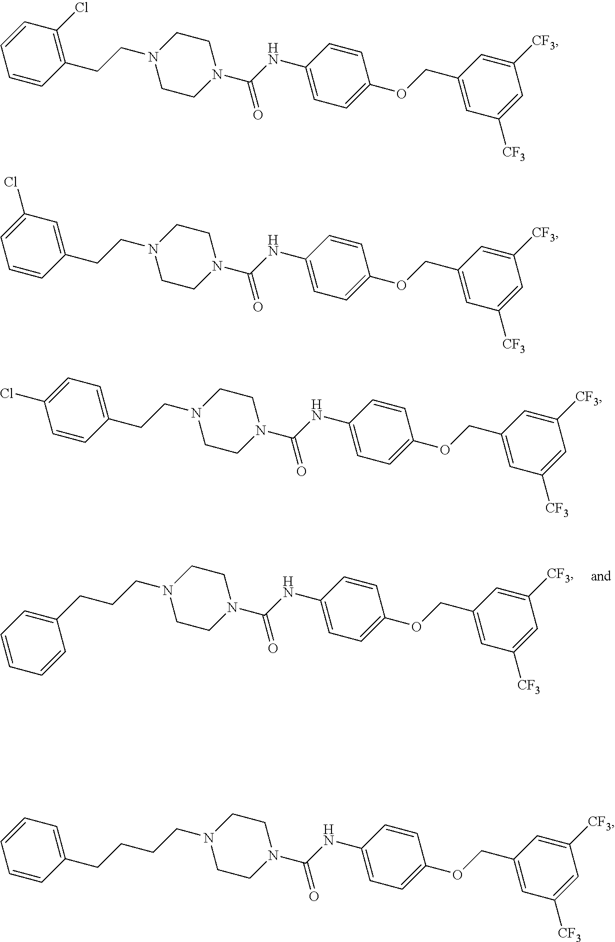

or a pharmaceutically acceptable salt or derivative thereof.

The present disclosure also includes compounds described herein with at least one desired isotopic substitution of an atom, at an amount above the natural abundance of the isotope, i.e., enriched.

Examples of isotopes that can be incorporated into compounds of the present disclosure include isotopes of hydrogen, carbon, nitrogen, oxygen, phosphorous, fluorine, and chlorine, such as ²H, ³H, ¹C, ¹³C, ¹⁵N, ¹⁷O, ¹⁸O, ¹⁸F, ³¹P, ³²P, ³⁵S, ³⁶Cl, and ¹²⁵I, respectively. In one aspect, isotopically labeled compounds can be used in metabolic studies (with ¹⁴C), reaction kinetic studies (with, for example ²H or ³H), detection or imaging techniques, such as positron emission tomography (PET) or single-photon emission computed tomography (SPECT) including drug and substrate tissue distribution assays, or in radioactive treatment of patients. In particular, an ¹⁸F labeled compound may be particularly desirable for PET or SPECT studies. Isotopically labeled compounds of this disclosure and prodrugs thereof can generally be prepared by carrying out the procedures disclosed herein by substituting a readily available isotopically labeled reagent for a non-isotopically labeled reagent.

By way of general example and without limitation, isotopes of hydrogen, for example deuterium (²H) and tritium (³H) may optionally be used anywhere in described structures that achieves the desired result. Alternatively or in addition, isotopes of carbon, e.g., ¹³C and ¹⁴C, may be used. In one aspect, the isotopic substitution is replacing hydrogen with a deuterium at one or more locations on the molecule to improve the performance of the molecule as a drug, for example, the pharmacodynamics, pharmacokinetics, biodistribution, half-life, stability, AUC, Tₘₐₓ, Cₘₐₓ, etc. For example, the deuterium can be bound to carbon in allocation of bond breakage during metabolism (an alpha-deuterium kinetic isotope effect) or next to or near the site of bond breakage (a beta-deuterium kinetic isotope effect).

Isotopic substitutions, for example deuterium substitutions, can be partial or complete. Partial deuterium substitution means that at least one hydrogen is substituted with deuterium. In certain aspects, the isotope is 80, 85, 90, 95, or 99% or more enriched in an isotope at any location of interest. In some aspects, deuterium is 80, 85, 90, 95, or 99% enriched at a desired location. Unless otherwise stated, the enrichment at any point is above natural abundance, and in an aspect is enough to alter a detectable property of the compounds as a drug in a human.

The compounds of the present disclosure may form a solvate with solvents (including water). Therefore, in one aspect, this disclosure includes a solvated form of the active compound. The term “solvate” refers to a molecular complex of a compound of the present invention (including a salt thereof) with one or more solvent molecules. Non-limiting examples of solvents are water, ethanol, dimethyl sulfoxide, acetone and other common organic solvents. The term “hydrate” refers to a molecular complex comprising a disclosed compound and water. Pharmaceutically acceptable solvates in accordance with this disclosure include those wherein the solvent of crystallization may be isotopically substituted, e.g., D₂O, d₆-acetone, or d₆-DMSO. A solvate can be in a liquid or solid form.

A“prodrug” as used herein means a compound which when administered to a host in vivo is converted into a parent drug. As used herein, the term “parent drug” means any of the presently described compounds herein. Prodrugs can be used to achieve any desired effect, including to enhance properties of the parent drug or to improve the pharmaceutic or pharmacokinetic properties of the parent, including to increase the half-life of the drug in vivo. Prodrug strategies provide choices in modulating the conditions for in vivo generation of the parent drug. Non-limiting examples of prodrug strategies include covalent attachment of removable groups, or removable portions of groups, for example, but not limited to, acylating, phosphorylation, phosphonylation, phosphoramidate derivatives, amidation, reduction, oxidation, esterification, alkylation, other carboxy derivatives, sulfoxy or sulfone derivatives, carbonylation, or anhydrides, among others. In certain aspects, the prodrug renders the parent compound more lipophilic. In certain aspects, a prodrug can be provided that has several prodrug moieties in a linear, branched, or cyclic manner. For example, non-limiting aspects include the use of a divalent linker moiety such as a dicarboxylic acid, amino acid, diamine, hydroxycarboxylic acid, hydroxyamine, di-hydroxy compound, or other compound that has at least two functional groups that can link the parent compound with another prodrug moiety, and is typically biodegradable in vivo. In some aspects, 2, 3, 4, or 5 prodrug biodegradable moieties are covalently bound in a sequence, branched, or cyclic fashion to the parent compound. Non-limiting examples of prodrugs according to the present disclosure are formed with: a carboxylic acid on the parent drug and a hydroxylated prodrug moiety to form an ester; a carboxylic acid on the parent drug and an amine prodrug to form an amide; an amino on the parent drug and a carboxylic acid prodrug moiety to form an amide; an amino on the parent drug and a sulfonic acid to form a sulfonamide; a sulfonic acid on the parent drug and an amino on the prodrug moiety to form a sulfonamide; a hydroxyl group on the parent drug and a carboxylic acid on the prodrug moiety to form an ester; a hydroxyl on the parent drug and a hydroxylated prodrug moiety to form an ester; a phosphonate on the parent drug and a hydroxylated prodrug moiety to form a phosphonate ester; a phosphoric acid on the parent drug and a hydroxylated prodrug moiety to form a phosphate ester; a hydroxyl on the parent drug and a phosphonate on the prodrug to form a phosphonate ester; a hydroxyl on the parent drug and a phosphoric acid prodrug moiety to form a phosphate ester; a carboxylic acid on the parent drug and a prodrug of the structure HO—(CH₂)₂—O—(C₂₋₂₄ alkyl) to form an ester; a carboxylic acid on the parent drug and a prodrug of the structure HO—(CH₂)₂—S—(C₂₋₂₄ alkyl) to form a thioester; a hydroxyl on the parent drug and a prodrug of the structure HO—(CH₂)₂—O—(C₂₋₂₄ alkyl) to form an ether; a hydroxyl on the parent drug and a prodrug of the structure HO—(CH₂)₂—O—(C₂₋₂₄ alkyl) to form an thioether, and a carboxylic acid, oxime, hydrazide, hydrazine, amine or hydroxyl on the parent compound and a prodrug moiety that is a biodegradable polymer or oligomer including but not limited to polylactic acid, polylactide-co-glycolide, polyglycolide, polyethylene glycol, polyanhydride, polyester, polyamide, or a peptide.

In some aspects, a prodrug is provided by attaching a natural or non-natural amino acid to an appropriate functional moiety on the parent compound, for example, oxygen, nitrogen, or sulfur, and typically oxygen or nitrogen, usually in a manner such that the amino acid is cleaved in vivo to provide the parent drug. The amino acid can be used alone or covalently linked (straight, branched or cyclic) to one or more other prodrug moieties to modify the parent drug to achieve the desired performance, such as increased half-life, lipophilicity, or other drug delivery or pharmacokinetic properties. The amino acid can be any compound with an amino group and a carboxylic acid, which includes an aliphatic amino acid, alkyl amino acid, aromatic amino acid, heteroaliphatic amino acid, heteroalkyl amino acid, heterocyclic amino acid, or heteroaryl amino acid.

### Pharmaceutical Compositions

The compounds described herein can be administered by any suitable method and technique presently or prospectively known to those skilled in the art. For example, the active components described herein can be formulated in a physiologically- or pharmaceutically-acceptable form and administered by any suitable route known in the art including, for example, oral and parenteral routes of administering. As used herein, the term “parenteral” includes subcutaneous, intradermal, intravenous, intramuscular, intraperitoneal, and intrasternal administration, such as by injection. Administration of the active components of their compositions can be a single administration, or at continuous and distinct intervals as can be readily determined by a person skilled in the art.

Compositions, as described herein, comprising an active compound and a pharmaceutically acceptable carrier or excipient of some sort may be useful in a variety of medical and non-medical applications. For example, pharmaceutical compositions comprising an active compound and an excipient may be useful for the treatment or prevention of an infection with a parasitic protozoa in a subject in need thereof.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Excipients” include any and all solvents, diluents or other liquid vehicles, dispersion or suspension aids, surface active agents, isotonic agents, thickening or emulsifying agents, preservatives, solid binders, lubricants and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes: oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition or cosmetic composition, the choice of the excipient will depend on the route of administration, the agent being delivered, time course of delivery of the agent, etc., and can be administered to humans and/or to animals, orally, rectally, parenterally, intracisternally, intravaginally, intranasally, intraperitoneally, topically (as by powders, creams, ointments, or drops), buccally, or as an oral or nasal spray. In some aspects, the active compounds disclosed herein are administered topically.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabinogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain aspects, the preservative is an anti-oxidant. In other aspects, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Additionally, the composition may further comprise a polymer. Exemplary polymers contemplated herein include, but are not limited to, cellulosic polymers and copolymers, for example, cellulose ethers such as methylcellulose (MC), hydroxyethylcellulose (HEC), hydroxypropyl cellulose (HPC), hydroxypropyl methyl cellulose (HPMC), methylhydroxyethylcellulose (MHEC), methylhydroxypropylcellulose (MHPC), carboxymethyl cellulose (CMC) and its various salts, including, e.g., the sodium salt, hydroxyethylcarboxymethylcellulose (HECMC) and its various salts, carboxymethylhydroxyethylcellulose (CMHEC) and its various salts, other polysaccharides and polysaccharide derivatives such as starch, dextran, dextran derivatives, chitosan, and alginic acid and its various salts, carageenan, various gums, including xanthan gum, guar gum, gum arabic, gum karaya, gum ghatti, konjac and gum tragacanth, glycosaminoglycans and proteoglycans such as hyaluronic acid and its salts, proteins such as gelatin, collagen, albumin, and fibrin, other polymers, for example, polyhydroxyacids such as polylactide, polyglycolide, polyl(lactide-co-glycolide) and poly(.epsilon.-caprolactone-co-glycolide)-, carboxyvinyl polymers and their salts (e.g., carbomer), polyvinylpyrrolidone (PVP), polyacrylic acid and its salts, polyacrylamide, polyacrylic acid/acrylamide copolymer, polyalkylene oxides such as polyethylene oxide, polypropylene oxide, poly(ethylene oxide-propylene oxide), and a Pluronic polymer, polyoxy ethylene (polyethylene glycol), polyanhydrides, polyvinylalchol, polyethyleneamine and polypyrridine, polyethylene glycol (PEG) polymers, such as PEGylated lipids (e.g., PEG-stearate, 1,2-Distearoyl-sn-glycero-3-Phosphoethanolamine-N-[Methoxy(Polyethylene glycol)-1000], 1,2-Distearoyl-sn-glycero-3-Phosphoethanolamine-N-[Methoxy(Polyethylene glycol)-2000], and 1,2-Distearoyl-sn-glycero-3-Phosphoethanolamine-N-[Methoxy(Polyethylene glycol)-5000]), copolymers and salts thereof.

Additionally, the composition may further comprise an emulsifying agent. Exemplary emulsifying agents include, but are not limited to, a polyethylene glycol (PEG), a polypropylene glycol, a polyvinyl alcohol, a poly-N-vinyl pyrrolidone and copolymers thereof, poloxamer nonionic surfactants, neutral water-soluble polysaccharides (e.g., dextran, Ficoll, celluloses), non-cationic poly(meth)acrylates, non-cationic polyacrylates, such as poly (meth) acrylic acid, and esters amide and hydroxy alkyl amides thereof, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. In certain aspects, the emulsifying agent is cholesterol.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be a injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain aspects, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Compositions for rectal or vaginal administration may be in the form of suppositories which can be prepared by mixing the particles with suitable non-irritating excipients or carriers such as cocoa butter, polyethylene glycol, or a suppository wax which are solid at ambient temperature but liquid at body temperature and therefore melt in the rectum or vaginal cavity and release the particles.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Tablets, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

### Methods of Use

The compounds and compositions disclosed herein may also find use in treating infections caused by parasitic protozoa, for example infections caused by a Leishmania or Trypanosoma parasite.

Thus, in one aspect, a method is provided of treating an infection with a parasitic protozoa in a subject in need thereof comprising administering to the subject a therapeutically effective amount of a compound described herein, or a pharmaceutically acceptable salt or derivative thereof, or a pharmaceutical composition thereof.

In another aspect, a method is provided of preventing an infection with a parasitic protozoa in a subject in need thereof comprising administering to the subject a therapeutically effective amount of a compound described herein, or a pharmaceutically acceptable salt or derivative thereof, or a pharmaceutical composition thereof.

In some aspects, the parasitic protozoa expresses CYP51 and/or CYP5122A1.

In some aspects, the parasitic protozoa comprises a Leishmania parasite.

In some aspects, the Leishmania parasite comprises L. aethiopica, L. amaronensis, L. arabica, L. archibaldi, L. aristedesi, L. viannia, L. braziliensis, L. chagasi, L. colombiensis, L. deanei, L. donovani, L. enriettii, L. equatorensis, L. forattinii, L. garnhami, L. gerbili, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tropica, L. turanica, or L. venezuelensis.

In some aspects, the infection comprises cutaneous leishmaniasis, mucocutaneous leishmaniasis, or visceral leishmaniasis. Leishmaniasis is a neglected tropical disease that presents a wide array of clinical manifestations. Leishmaniasis is caused by parasites of the genus Leishmania. It is typically spread through the bite of phlebotomine sandflies of the genera Phlebotomurs and Lutzomyia and occurs most frequently in the tropics and sub-tropics of Africa, Asia, the Americas, and southern Europe. The symptoms of leishmaniasis are skin sores which erupt weeks to months after the person is bitten by infected sand flies. Leishmania may be divided into three types. Cutaneous leishmaniasis is the most common form, which causes an open sore at the bite sites which heals in a few months to a year and a half, leaving an unpleasant-looking scar. Diffuse cutaneous leishmaniasis produces widespread lesions which resemble leprosy and may not heal on its own. Mucocutaneous leishmaniasis causes both skin and mucosal ulcers with damage primarily of the nose and mouth. Visceral leishmaniasis is the most serious form and is generally fatal if untreated, involving migration of the parasite to internal organs such as the liver, spleen, and bone marrow.

In some aspects, the parasitic protozoa comprises a Trypanosoma parasite.

In some aspects, the Trypanosoma parasite comprises T. ambystomae, T. avium, T. boissoni, T. brucei, T. cruzi, T. congolense, T. equimim, T equiperdum, T. evansi, T. everetti, T. hosei, T. irwini, T. lewisi, T. melophagium, T. paddae, T. parroti, T. percae, T. rangeli, T. rotatorium, T. rugosae, T. sergenti, T. simiae, T. simpercae, T. suis, T. theileri, T. triglae, or T. vivax.

In some aspects, the infection comprises African trypanosomiasis, Chagas disease, nagana, and surra.

African trypanosomiasis, also known African sleeping sickness or simply sleeping sickness, is a parasite infection of humans and other animals caused by T. brucei transmitted by the bite of an infected tsetse fly. The first stage of the disease is characterized by fever, headaches, itchiness, and joint pains, beginning one to three weeks after the bite. Weeks to months later, the second stage begins with confusion, poor coordination, numbness, and trouble sleeping. If the disease is not treated quickly it can lead to death.

Chagas disease, known as American trypanosomiasis, is a tropical parasitic disease caused by T. cruzi that spreads to humans and other mammals by the bit of insects in the subfamily Triatominae, known as “kissing bugs.” The symptoms change over the course of the infection. In the early stage, symptoms are typically either not present or mild, and may include fever, swollen lymph nodes, headaches, or swelling at the site of the bite. After four to eight weeks, untreated individuals enter the chronic phase of disease, which in most cases does not result in further symptoms. Up to 45% of people with chronic infections develop heart disease 10-30 years after the initial illness, which can lead to heart failure. Digestive complications, including an enlarged esophagus or an enlarged colon, may also occur in up to 21% of people, and up to 10/o of people may experience nerve damage.

Nagana, also known as animal trypanosomiasis, is a disease of vertebrates caused by trypanosomes of several species in the genus Trypanosoma, such as T. brucei. Trypanosoma vivax causes nagana mainly in West Africa, although it has spread to South America. The trypanosomes are transmitted by tsetse flies. The incubation period ranges from 4 days to approximately 8 weeks. The infection leads to significant weight loss and anemia. Various symptoms are observed, including fever, edema, adenitis, dermatitis, and nervous disorders. In some animals, the disease is fatal unless treated.

Surra is a disease of vertebrate animals caused by protozoan trypanosomes, particularly T. evansi, which infect the blood of the vertebrate host, causing fever, weakness, and lethargy which lead to weight loss and anemia. An acute form of the disease, which is generally fatal unless treated, occurs in horses, donkeys, mules, cattle, buffalo, deer, camels, llamas, dogs, and cats. This form is caused by T. evansi and is transmitted by horse-flies and the vampire bat (Desmodus rotundus). This form occurs in South America, Northern Africa, and the Middle East. A chronic form of the disease, which is milder but persistent, occurs in pigs, sheep, and goats. This form is caused by T. suis and is transmitted by tsetse flies and occurs only in Africa.

In some aspects, the subject is a human, dog, cat, cow, horse, sheep, pig, bird, amphibian, or fish.

The active ingredient may be administered in such amounts, time, and route deemed necessary in order to achieve the desired result. The exact amount of the active ingredient will vary from subject to subject, depending on the species, age, and general condition of the subject, the severity of the medical disorder, the particular active ingredient, its mode of administration, its mode of activity, and the like. The active ingredient, whether the active compound itself, or the active compound in combination with an agent, is preferably formulated in dosage unit form for ease of administration and uniformity of dosage. It will be understood, however, that the total daily usage of the active ingredient will be decided by the attending physician within the scope of sound medical judgment. The specific therapeutically effective dose level for any particular subject will depend upon a variety of factors including the disorder being treated and the severity of the disorder; the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The active ingredient may be administered by any route. In some aspects, the active ingredient is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, enteral, sublingual; by intratracheal instillation, bronchial instillation, and/or inhalation, and/or as an oral spray, nasal spray, and/or aerosol. In general, the most appropriate route of administration will depend upon a variety of factors including the nature of the active ingredient (e.g., its stability in the environment of the gastrointestinal tract), the condition of the subject (e.g., whether the subject is able to tolerate oral administration), etc.

The exact amount of an active ingredient required to achieve a therapeutically or prophylactically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the active agents and pharmaceutical compositions disclosed herein can be determined by comparing their in vitro activity, and in vivo activity in animal models. Methods for the extrapolation of effective dosages in mice, and other animals, to humans are known to the art.

To provide for the administration of such dosages for the desired therapeutic treatment, in some aspects, pharmaceutical compositions disclosed herein can comprise between 0.1% and 45%, and especially, 1 and 15%, by weight of the total of one or more of the compounds based on the weight of the total composition including carriers and/or diluents. Illustratively, dosage levels of the administered active ingredients can be: intravenous 0.01 to about 20 mg/kg; intraperitoneal, 0.01 to about 100 mg/kg; subcutaneous, 0.01 to about 100 mg/kg; intramuscular, 0.01 to about 100 mg/kg; orally 0.01 to about 200 mg/kg, and preferably about 1 to 100 mg/kg; intranasally, 0.01 to about 20 mg/kg; and aerosol, 0.01 to about 20 mg/kg of animal (body) weight.

### Kits

Kits for practicing the methods described herein are further provided. By “kit” is intended any manufacture (e.g., a package or a container) comprising at least one reagent, e.g., any one of the compounds described herein. The kit can be promoted, distributed, or sold as a unit for performing the methods described herein. Additionally, the kits can contain a package insert describing the kit and methods for its use. Any or all of the kit reagents can be provided within containers that protect them from the external environment, such as in sealed containers or pouches.

Also disclosed are kits that comprise a composition comprising a compound disclosed herein in one or more containers. The disclosed kits can optionally include pharmaceutically acceptable carriers and/or diluents. In one aspect, a kit includes one or more other components, adjuncts, or adjuvants as described herein. In another aspect, a kit includes one or more antiparasitic agents, such as those agents described herein. In one aspect, a kit includes instructions or packaging materials that describe how to administer a compound or composition of the kit. Containers of the kit can be of any suitable material, e.g., glass, plastic, metal, etc., and of any suitable size, shape, or configuration. In one aspect, a compound and/or agent disclosed herein is provided in the kit as a solid, such as a tablet, pill, or powder form. In another aspect, a compound and/or agent disclosed herein is provided in the kit as a liquid or solution. In one aspect, the kit comprises an ampoule or syringe containing a compound and/or agent disclosed herein in liquid or solution form.

In view of the described compounds, compositions, and methods, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.
Aspect 1. A compound of Formula I

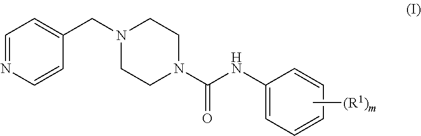

or a pharmaceutically acceptable salt or derivative thereof;
wherein:
R¹ is selected from halo, cyano, azido, C₁-C₆ alkyl, C₁-C₆ haloalkyl, C₂-C₆ alkenyl, C₂-C₆ alkynyl, (C₃-C₆ cycloalkyl)-(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N)C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{x}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;
m is 0, 1, 2, 3, 4, or 5;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, and (5- to 10-membered monocyclic or bicyclic heteroaryl)-(C₀-C₀ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃—C-cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol;
with the proviso that

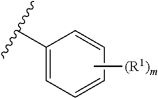

cannot be 3,5-dimethylphenyl.
Aspect 2. A compound of aspect 1, or a pharmaceutically acceptable salt or derivative thereof, wherein m is 0.
Aspect 3. A compound of aspect 1, or a pharmaceutically acceptable salt or derivative thereof, wherein m is 1.
Aspect 4. A compound of aspect 1, or a pharmaceutically acceptable salt or derivative thereof, wherein m is 2.
Aspect 5. A compound of aspect 1, or a pharmaceutically acceptable salt or derivative thereof, wherein m is 3.
Aspect 6. A compound of aspect 1, or a pharmaceutically acceptable salt or derivative thereof, wherein m is 4.
Aspect 7. A compound of aspect 1, or a pharmaceutically acceptable salt or derivative thereof, wherein m is 5.
Aspect 8. A compound of any one of aspects 1-7, or a pharmaceutically acceptable salt or derivative thereof, wherein R¹ is independently selected at each occurrence from halo, C₁-C₆ alkyl, C₁-C₆ alkoxy, C₁-C₆ haloalkyl, and C₁-C₆ haloalkoxy.
Aspect 9. A compound of any one of aspects 1-7, or a pharmaceutically acceptable salt or derivative thereof, wherein R¹ is independently selected at each occurrence from methyl, ethyl, propyl, isopropyl, butyl, tert-butyl, methoxy, ethoxy, propoxy, isopropoxy, tert-butoxy, fluoro, chloro, bromo, trifluoromethyl, and trifluoromethoxy.
Aspect 10. A compound of aspect 1, or a pharmaceutically acceptable salt or derivative thereof, wherein

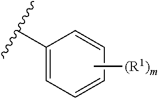

is selected from:

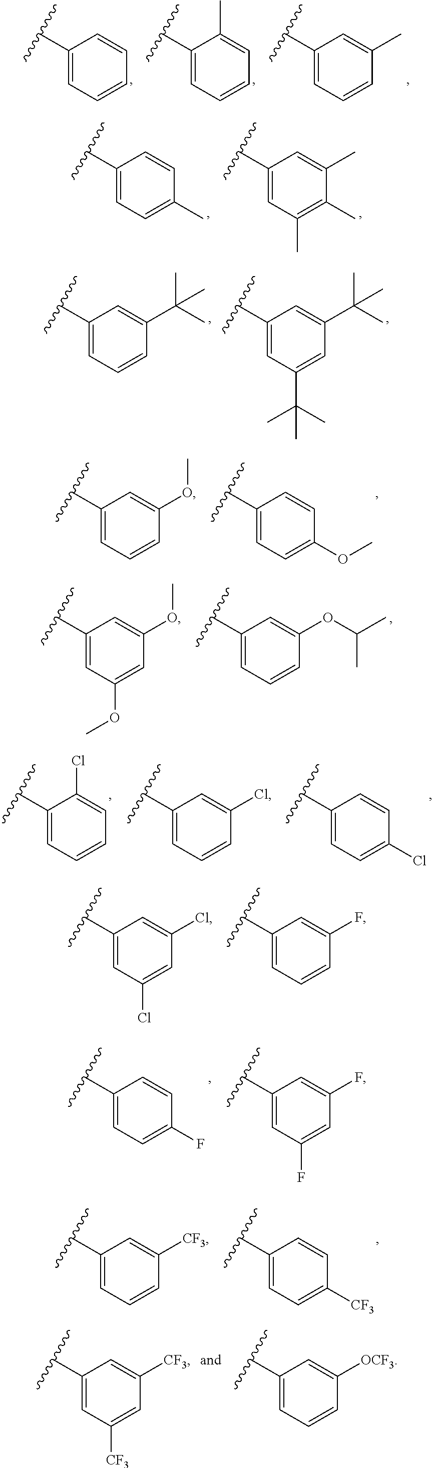

Aspect 11. A compound selected from:

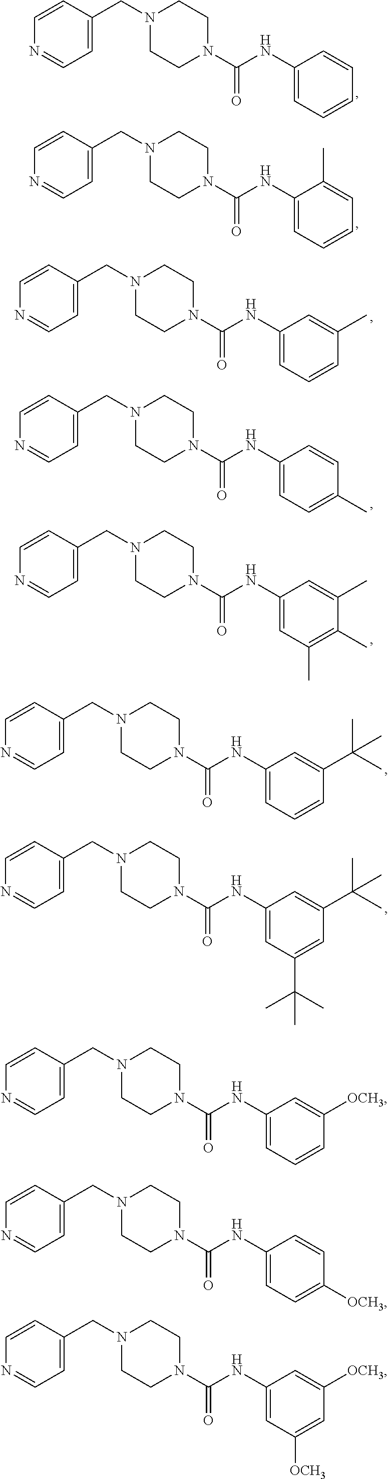

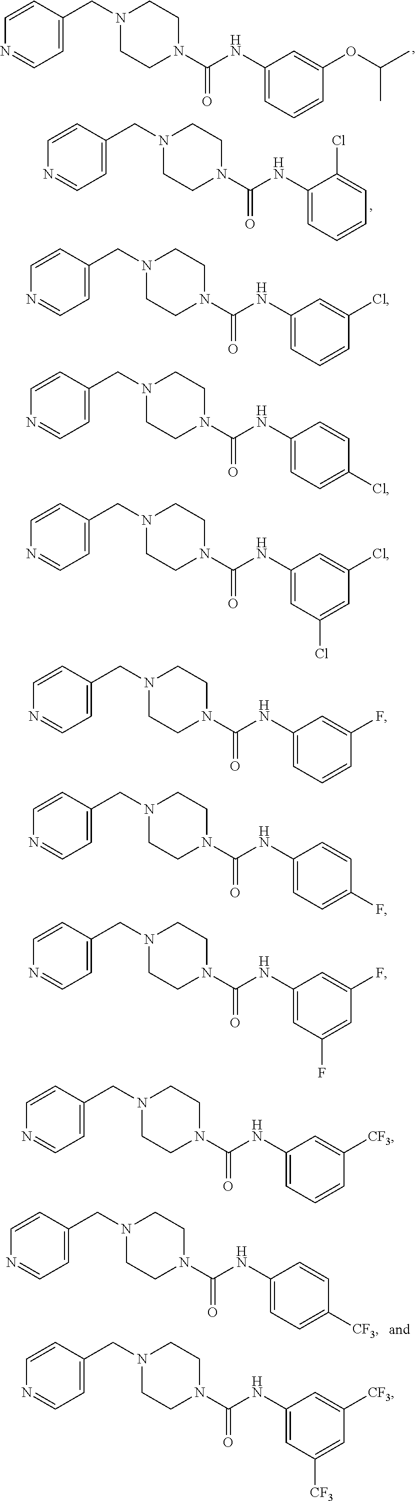

or a pharmaceutically acceptable salt or derivative thereof.
Aspect 12. A compound of Formula II

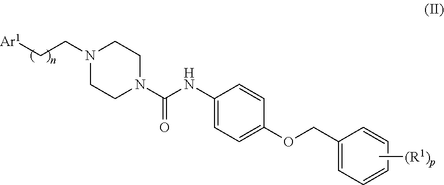

or a pharmaceutically acceptable salt thereof;
wherein:
R¹ is selected from halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;
n is 0, 1, or 2;
p is 1, 2, 3, 4, or 5;
Ar¹ is 6- to 10-membered monocyclic or bicyclic aryl or 5- to 10-membered monocyclic or bicyclic heteroaryl, wherein Ar¹ is optionally substituted with one or more Z groups as allowed by valency;
Z is selected from hydrogen, halo, cyano, azido, C₁-C₆ alkyl, C₁-C₆ haloalkyl, C₂-C₆ alkenyl, C₂-C₆ alkynyl, (C₃-C₆ cycloalkyl)(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{x}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{x}S(O)—(C₀-C₃ alkyl)-, and R^{x}S(O)₂—(C₀-C₃ alkyl)-;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, and (5- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.
Aspect 13. A compound of aspect 12, or a pharmaceutically acceptable salt or derivative thereof, wherein Ar¹ is phenyl optionally substituted with one or more (for example 1, 2, 3, 4, or 5) Z groups.
Aspect 14. A compound of aspect 12, or a pharmaceutically acceptable salt or derivative thereof, wherein Ar¹ is 5- to 6-membered monocyclic heteroaryl optionally substituted with one or more (for example, 1, 2, 3, or 4) Z groups.
Aspect 15. A compound of aspect 12, or a pharmaceutically acceptable salt or derivative thereof, wherein Ar¹ is selected from:

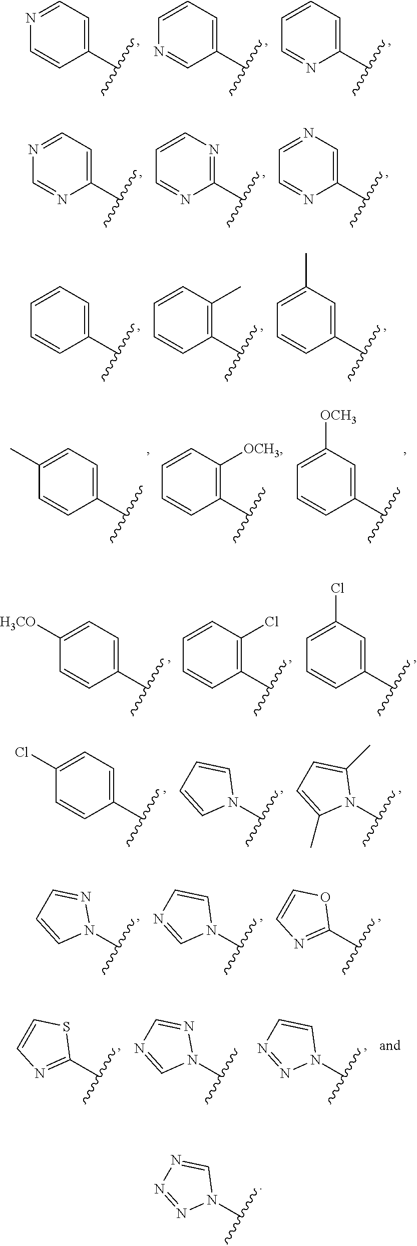

Aspect 16. A compound of any one of aspects 12-15, or a pharmaceutically acceptable salt or derivative thereof, wherein n is 0.
Aspect 17. A compound of any one of aspects 12-15, or a pharmaceutically acceptable salt or derivative thereof, wherein n is 1.
Aspect 18. A compound of any one of aspects 12-15, or a pharmaceutically acceptable salt or derivative thereof, wherein n is 2.
Aspect 19. A compound of any one of aspects 12-18, or a pharmaceutically acceptable salt or derivative thereof, wherein p is 1.
Aspect 20. A compound of any one of aspects 12-18, or a pharmaceutically acceptable salt or derivative thereof, wherein p is 2.
Aspect 21. A compound of any one of aspects 12-18, or a pharmaceutically acceptable salt or derivative thereof, wherein p is 3.
Aspect 22. A compound of any one of aspects 12-18, or a pharmaceutically acceptable salt or derivative thereof, wherein p is 4.
Aspect 23. A compound of any one of aspects 12-18, or a pharmaceutically acceptable salt or derivative thereof, wherein p is 5.
Aspect 24. A compound of any one of aspects 12-23, or a pharmaceutically acceptable salt or derivative thereof, wherein R¹ is independently selected at each occurrence from halo, C₁-C₆ alkyl, C₁-C₆ alkoxy, C₁-C₆ haloalkyl, and C₁-C₆ haloalkoxy.
Aspect 25. A compound of any one of aspects 12-23, or a pharmaceutically acceptable salt or derivative thereof, wherein R¹ is independently selected at each occurrence from methyl, ethyl, propyl, isopropyl, butyl, tert-butyl, methoxy, ethoxy, propoxy, isopropoxy, tert-butoxy, fluoro, chloro, bromo, trifluoromethyl, and trifluoromethoxy.
Aspect 26. A compound of any one of aspects 12-18, or a pharmaceutically acceptable salt or derivative thereof, wherein

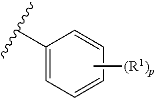

is selected from:

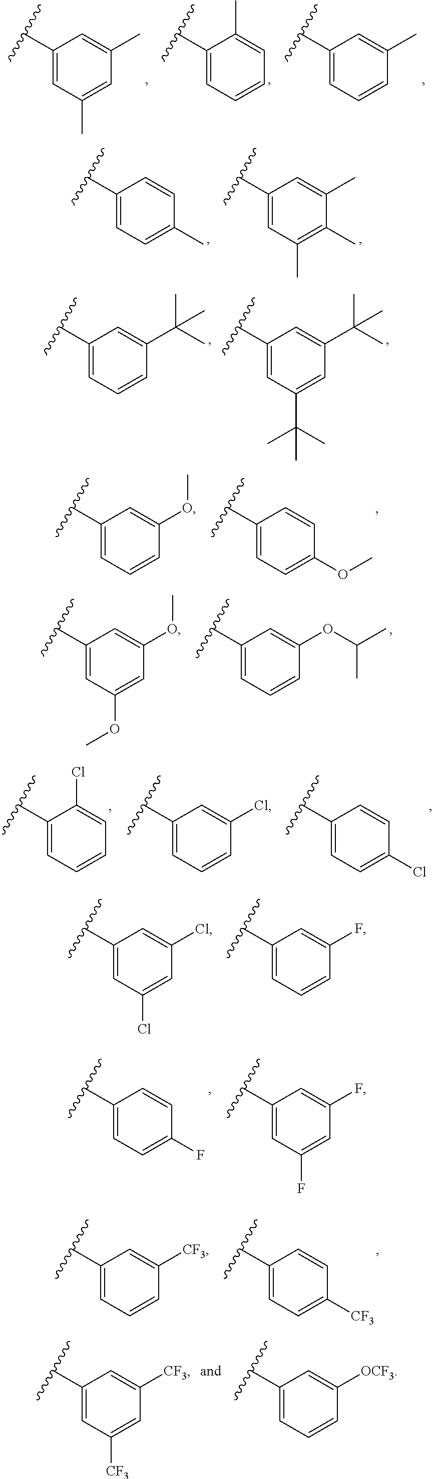

Aspect 27. A compound selected from:

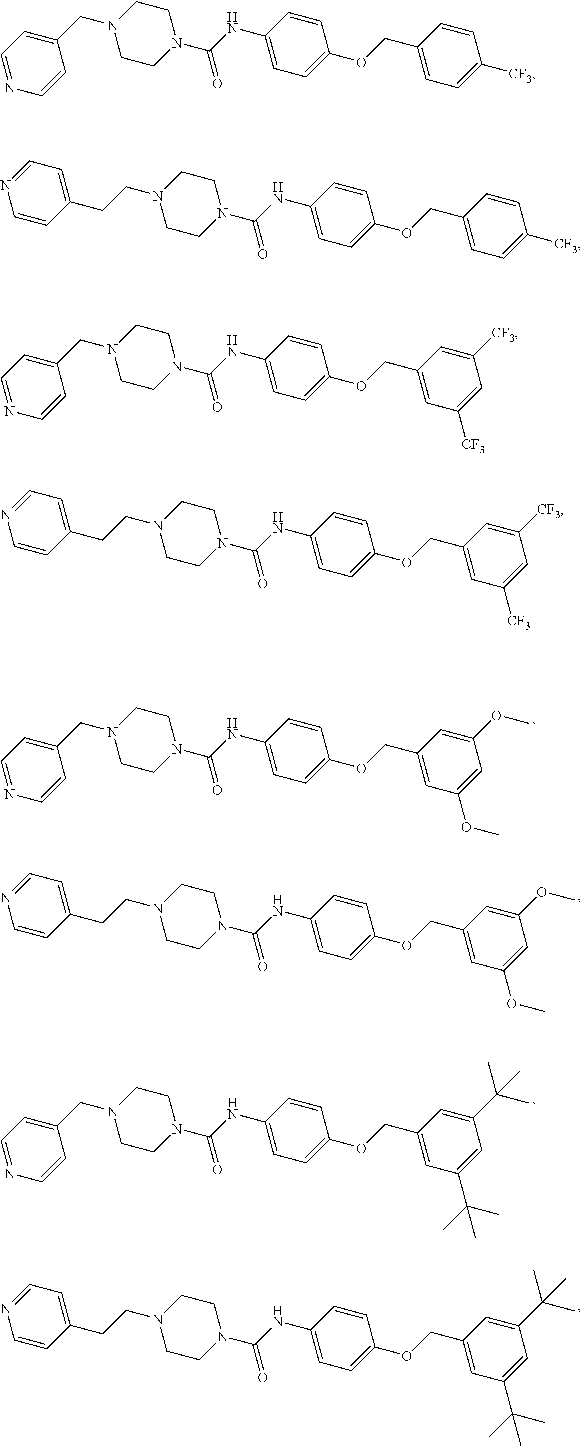

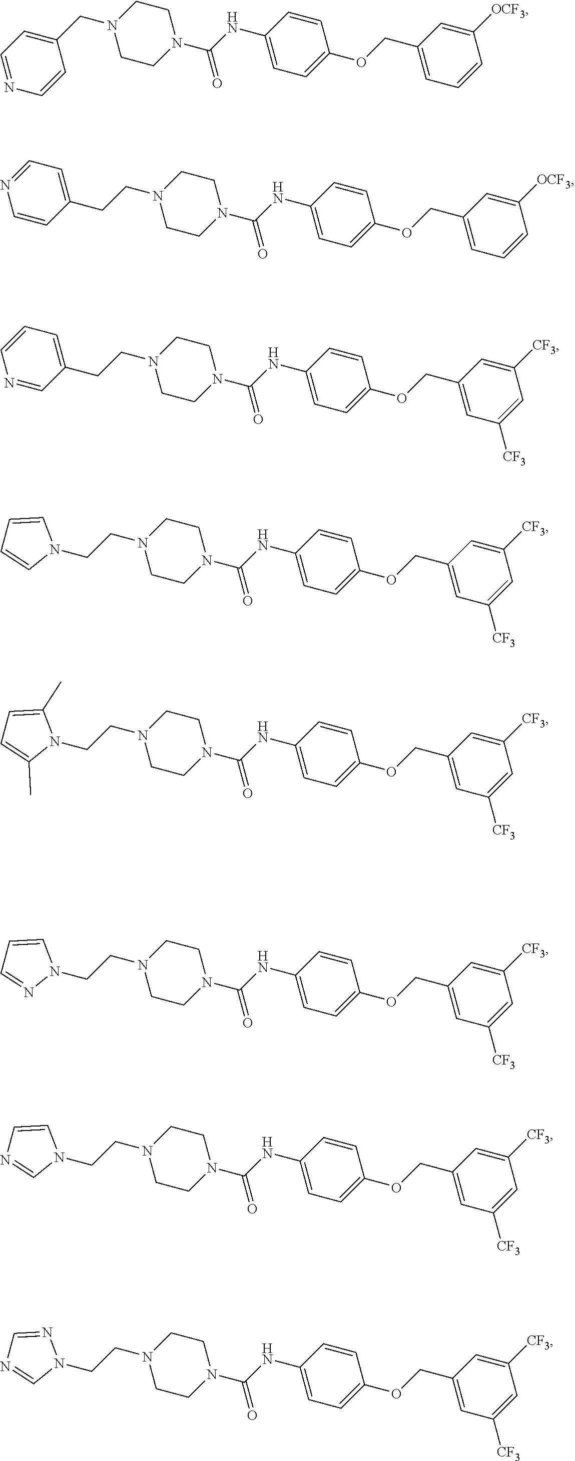

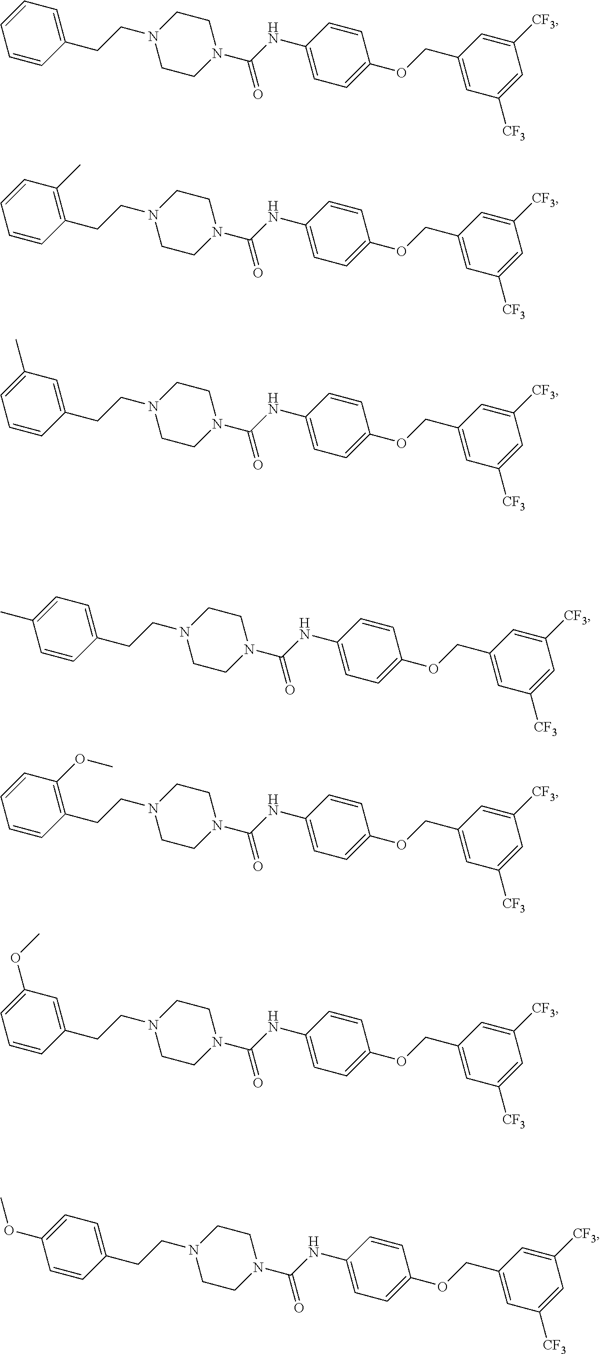

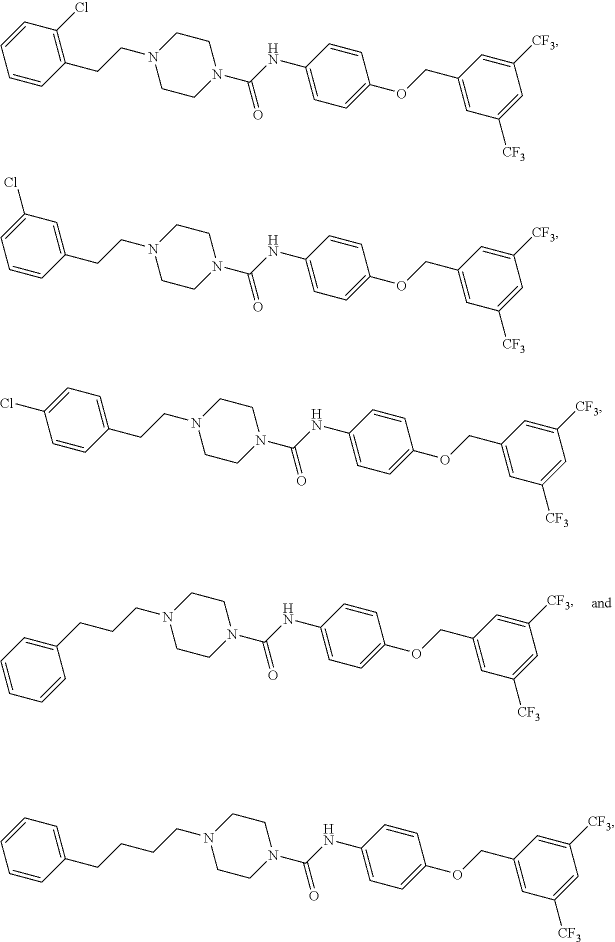

or a pharmaceutically acceptable salt or derivative thereof.
Aspect 28. A pharmaceutical composition comprising a compound of any one of aspects 1-27, or a pharmaceutically acceptable salt or derivative thereof, and a pharmaceutically acceptable carrier or excipient.
Aspect 29. A method of treating an infection with a parasitic protozoa in a subject in need thereof comprising administering to the subject a therapeutically effective amount of a compound of any one of aspects 1-27, or a pharmaceutically acceptable salt or derivative thereof, or a pharmaceutical composition of aspect 28.
Aspect 30. A method of preventing an infection with a parasitic protozoa in a subject in need thereof comprising administering to the subject a therapeutically effective amount of a compound of any one of aspects 1-27, or a pharmaceutically acceptable salt or derivative thereof, or a pharmaceutical composition of aspect 28.
Aspect 31. The method of aspect 28 or 29, wherein the parasitic protozoa expresses CYP51 and/or CYP5122A1.
Aspect 32. The method of any one of aspects 28-31, wherein the parasitic protozoa comprises a Leishmania parasite.
Aspect 33. The method of aspect 32, wherein the Leishmania parasite comprises L. aethiopica, L. amazonensis, L. arabica, L. archibaldi, L. aristedesi, L. viannia, L. braziliensis, L. chagasi, L. colombiensis, L. deanei, L. donovani, L. enriettii, L. equatorensis, L. forattinii, L. garnhami, L. gerbili, L. guyanensis, L. herreri, L. hertigi, L. infantum, L. killicki, L. lainsoni, L. major, L. Mexicana, L. naiffi, L. panamensis, L. peruiana, L. pifanoi, L. shawi, L. tarentolae, L. tropica, L. turanica, or L. venezuelensis.
Aspect 34. The method of aspect 32 or 33, wherein the infection comprises cutaneous leishmaniasis.
Aspect 35. The method of aspect 32 or 33, wherein the infection comprises mucocutaneous leishmaniasis.
Aspect 36. The method of aspect 32 or 33, wherein the infection comprises visceral leishmaniasis.
Aspect 37. The method of any one of aspects 28-31, wherein the parasitic protozoa comprises a Trypanosoma parasite.
Aspect 38. The method of aspect 37, wherein the Trypanosoma parasite comprises T. ambystomae, T avium, T. boissoni, T brucei, T. cruzi, T. congolenrse, T. equinum, T. equiperdum, T evansi, T everetti, T. hosei, T. irwini, T. lewisi, T. melophagium, T. paddae, T. parroti, T. percae, T. rangeli, T. rolatorium, T. rugosae, T. sergenti, T. simiae, T. sinipercae, T. suis, T theileri, T. triglae, or T. vivax.
Aspect 39. The method of aspect 37 or 38, wherein the infection comprises African trypanosomiasis.
Aspect 40. The method of aspect 37 or 38, wherein the infection comprises Chagas disease.
Aspect 41. The method of aspect 37 or 38, wherein the infection comprises nagana.
Aspect 42. The method of aspect 37 or 38, wherein the infection comprises surra.
Aspect 43. The method of any one of aspects 28-42, wherein the subject is a human, dog, cat, cow, horse, sheep, pig, bird, amphibian, or fish.
Aspect 44. The method of any one of aspects 28-42, wherein the subject is a human.
Aspect 45. A compound of any one of aspects 1-27, or a pharmaceutically acceptable salt or derivative thereof, or a pharmaceutical composition of aspect 28, for use in treating an infection with a parasitic protozoa in a subject in need thereof.
Aspect 46. A compound of any one of aspects 1-27, or a pharmaceutically acceptable salt or derivative thereof, or a pharmaceutical composition of aspect 28, for use in treating an infection with a parasitic protozoa in a subject in need thereof.
Aspect 47. The compound or composition of aspect 45 or 46, wherein the parasitic protozoa expresses CYP51 and/or CYP5122A1.
Aspect 48. The compound or composition of any one of aspects 45-47, wherein the parasitic protozoa comprises a Leishmania parasite.
Aspect 49. The compound or composition of aspect 48, wherein the Leishmania parasite comprises L. aethiopica, L. amazonensis, L. arabica, L. archibaldi, L. aristedesi, L. viannia, L. braziliensis, L. chagasi, L. colombiensis, L. deantei, L. donovani, L. enriettii, L. equatorensis, L. foraltinii, L. garnhami, L. gerhili, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. larentolae, L. tropica, L. turanica, or L. venezuelensis.
Aspect 50. The compound or composition of aspect 48 or 49, wherein the infection comprises cutaneous leishmaniasis.
Aspect 51. The compound or composition of aspect 48 or 49, wherein the infection comprises mucocutaneous leishmaniasis.
Aspect 52. The compound or composition of aspect 48 or 49, wherein the infection comprises visceral leishmaniasis.
Aspect 53. The compound or composition of any one of aspects 45-47, wherein the parasitic protozoa comprises a Trypanosoma parasite.
Aspect 54. The compound or composition of aspect 53, wherein the Trypanosoma parasite comprises T. ambystomae, T. avium, T. boissoni, T. brucei, T. cruzi, T. congolense, T. equinum, 7 equiperdum, T. evansi, T everetti, T. hosei, T. irwini, T. lewisi, T melophagium, T. paddae, T parroti, T. percae, T. rangeli, T. rotatorium, T. rugosae, T. sergenti, T. simiae, T. sinipercae, T. suis, T. theileri, T. triglae, or T. vivax.
Aspect 55. The compound or composition of aspect 53 or 54, wherein the infection comprises African trypanosomiasis.
Aspect 56. The compound or composition of aspect 53 or 54, wherein the infection comprises Chagas disease.
Aspect 57. The compound or composition of aspect 53 or 54, wherein the infection comprises nagana.
Aspect 58. The compound or composition of aspect 53 or 54, wherein the infection comprises surra.
Aspect 59. The compound or composition of any one of aspects 45-58, wherein the subject is a human, dog, cat, cow, horse, sheep, pig, bird, amphibian, or fish.
Aspect 60. The compound or composition of any one of aspects 45-58, wherein the subject is a human.
Aspect 61. Use of a compound of any one of aspects 1-27, or a pharmaceutically acceptable salt or derivative thereof, in the manufacture of a medicament for treating an infection with a parasitic protozoa in a subject in need thereof.
Aspect 62. Use of a compound of any one of aspects 1-27, or a pharmaceutically acceptable salt or derivative thereof, in the manufacture of a medicament for treating an infection with a parasitic protozoa in a subject in need thereof.
Aspect 63. The use of aspect 61 or 62, wherein the parasitic protozoa expresses CYP51 and/or CYP5122A1.
Aspect 64. The use of any one of aspects 61-63, wherein the parasitic protozoa comprises a Leishmania parasite.
Aspect 65. The use of aspect 64, wherein the Leishmania parasite comprises L. aethiopica, L. amazonensis, L. arabica, L. archibaldi, L. aristedesi, L. viannia, L. braziliensis, L. chagasi, L. colombiensis, L. deanei, L. donovani, L. enriettii, L. equatorensis, L. forattinii, L. garnhami, L. gerbili, L. guyanensis, L. herreri, L. hertigi, L. infantum, L. killicki, L. lainsoni, L. major, L. Mexicana, L. naiffi, L. panamensis, L. peruviana, L. pifanoi, L. shawi, L. tarentolae, L. tropica, L. turanica, or L. venezuelensis.
Aspect 66. The use of aspect 64 or 65, wherein the infection comprises cutaneous leishmaniasis.
Aspect 67. The use of aspect 64 or 65, wherein the infection comprises mucocutaneous leishmaniasis.
Aspect 68. The use of aspect 64 or 65, wherein the infection comprises visceral leishmaniasis.
Aspect 69. The use of any one of aspects 61-63, wherein the parasitic protozoa comprises a Trypanosoma parasite.
Aspect 70. The use of aspect 69, wherein the Trypanosoma parasite comprises T. ambystomae, T. avium, T. boissoni, T brucei, T. cruzi, T. congolense, T. equinum, T. equiperdum, T evansi, T everetti, T. hosei, T. irwini, T. lewisi, T. melophagium, T. paddae, T. parroi, T. percae, T. rangeli, T. rolalorium, T. rugosae, T. sergenti, T. simiae, T. sinipercae, T. suis, T. theileri, T. triglae, or T. vivax.
Aspect 71. The use of aspect 69 or 70, wherein the infection comprises African trypanosomiasis.
Aspect 72. The use of aspect 69 or 70, wherein the infection comprises Chagas disease.
Aspect 73. The use of aspect 69 or 70, wherein the infection comprises nagana.
Aspect 74. The use of aspect 69 or 70, wherein the infection comprises surra.
Aspect 75. The use of any one of aspects 61-74, wherein the subject is a human, dog, cat, cow, horse, sheep, pig, bird, amphibian, or fish.
Aspect 76. The use of any one of aspects 61-74, wherein the subject is a human.

A number of aspects of the disclosure have been described. Nevertheless, it will be understood that various modifications may be made without departing from the spirit and scope of the invention. Accordingly, other aspects are within the scope of the following claims.

By way of non-limiting illustration, examples of certain aspects of the present disclosure are given below.

### EXAMPLES

The following examples are put forth so as to provide those of ordinary skill in the art with a complete disclosure and description of how the compounds, compositions, a and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in degrees Celsius or is at ambient temperature, and pressure is at or near atmospheric pressure.

### Example 1. Pyridin-4-ylalkyl)piperazine-1-carboxamides and related compounds as Leishmania CYP51 and CYP5122A1 inhibitors

CYP5122A1, an enzyme involved in sterol biosynthesis in Leishmania, was recently characterized as a sterol C4-methyl oxidase. Screening of a library of compounds against CYP5122A1 and CYP51 from Leishmania resulted in the identification of two classes of N-phenyl-4-(pyridin-4-ylmethyl)piperazine-1-carboxamides as inhibitors of these enzymes. Analogs of screening hit N-(3,5-dimethylphenyl)-4-(pyridin-4-ylmethyl)piperazine-1-carboxamide (4a) were generally strong inhibitors of CYP51 but were less potent against CYP5122A1 and typically displayed weak inhibition of L. donovani promastigote growth. N-(4-(benzyloxy)phenyl)-4-(2-(pyridin-4-yl)alkyl)piperazine-1-carboxamides based on hit 18a were stronger inhibitors of both CYP5122A1 and L. donovani promastigote proliferation but also remained selective for inhibition of CYP51. Two compounds in this series, N-(4-((3,5-bis(trifluoromethyl)benzyl)oxy)phenyl)-4-(2-(pyridin-4-yl)ethyl)piperazine-1-carboxamide (18e) and N-(4-((3,5-di-tert-butylbenzyl)oxy)phenyl)-4-(2-(pyridin-4-yl)ethyl)piperazine-1-carboxamide (18i) showed selectivity for L. donovani compared to J774 macrophages and were effective against intracellular L. donovani with EC₅₀ values in the low micromolar range. Replacement of the 4-pyridyl ring present in 18e with imidazole resulted in a compound (4-(2-(1H-imidazol-1-yl)ethyl)-N-(4-((3,5-bis(trifluoromethyl)benzyl)oxy)phenyl)piperazine-1-carboxamide, 18p) with approximately fourfold selectivity for CYP5122A1 over CYP51 and inhibited both enzymes with IC₅₀ values≤1 μM, although selective potency against L. donovani promastigotes was lost.

### Introduction

Cytochrome P450 51 (CYP51), otherwise known as 14α-demethylase or ERG11 in fungi, is an enzyme that removes the methyl group at the 14 position of the tetracyclic steroid core as part of the sterol biosynthesis pathway. Inhibition of CYP51 family enzymes has long been known as a therapeutic strategy. In the treatment of fungal infections, drugs that inhibit CYP51 interfere with the formation of ergosterol, the end product of sterol biosynthesis in these organisms. Interruption of ergosterol biosynthesis in fungi alters the sterol composition of the membrane, leading to changes in permeability. Ergosterol is proposed to play a hormone-like role in yeast,¹ so inhibition of its biosynthesis may also interfere with other essential roles of ergosterol in these organisms. Imidazole- and triazole-based CYP51 inhibitors are important drugs for the treatment of both topical and systemic fungal infections.² These azole-based antifungal drugs are all believed to bind at the CYP51 active site through the coordination of iron at the heme prosthetic group through the lone pair of electrons present at the N3 atom of imidazole-based CYP51 inhibitors or the N4 atom of triazole-based CYP51 inhibitors.³⁻⁴ Given that CYP51 also carries out lanosterol demethylation in cholesterol biosynthesis in mammals and that mammals have a panel of related CYP enzymes that are critical for xenobiotic metabolism and other metabolic processes, the success of the azoles as antimicrobial drugs relies on their selective action on the fungal enzyme.

Since ergosterol and related sterols are also the end products of sterol biosynthesis in Leishmania and Trypanosoma spp, CYP51 has also been of interest as a target for treatment of the diseases caused by these kinetoplastid parasites. Posaconazole, a triazole-containing antifungal CYP51 inhibitor, was repurposed for use against Chagas disease based on the potency of this drug against T. cruzi in vitro and its outstanding efficacy in murine Chagas disease.⁵⁻⁶ Although activity was observed in posaconazole-treated patients presenting with chronic Chagas disease, significantly more patients in the posaconazole groups experienced treatment failure during follow-up compared to those in the benznidazole group.⁷ Azole antifungal drugs have been used for treating various types of cutaneous leishmaniasis⁸⁻⁹ and have also been evaluated in the past for efficacy against visceral leishmaniasis in humans,¹⁰⁻¹¹ but no azoles have gained approval for leishmaniasis treatment. Other candidate azoles that interact with kinetoplastid CYP51 have also been the subject of antiparasitic drug discovery efforts.¹²⁻¹⁷

In 2011, Verma et al. reported a CYP5122A1 protein from Leishmania donovani that is 22% identical to CYP51 in that organism.¹⁸ CYP5122A1 was required for the survival of these parasites and was shown to play a role in ergosterol biosynthesis. Parasites in which one CYP5122A1 allele was replaced with a selectable marker exhibited ergosterol levels that were 3.5-fold lower than in wild-type L. donovani promastigotes, while complementation with episomally expressed CYP5122A1 resulted in increased levels of ergosterol compared to the half knockout parasites.¹⁸ Recently, Wang et al. showed that CYP5122A1 is essential for both promastigotes and amastigotes of L. donovani and identified CYP5122A1 as a sterol C4-methyl oxidase.¹⁹ Stepwise oxidation of the C4 methyl groups is required for 4-demethylation in sterol biosynthesis.²⁰⁻²¹ Moreover, antifungal azole drugs such as posaconazole and clotrimazole exhibited dual inhibition of recombinant L. donovani CYP51 and CYP5122A1, while fluconazole and voriconazole displayed selective activity against L. donovani CYP51.¹⁹ Interestingly, the azole drugs that act as dual CYP51/CYP5122A1 inhibitors display strong in vitro antileishmanial activity, while fluconazole and voriconazole exhibit weak in vitro antileishmanial potency.¹⁹ Taken together, these data suggest that inhibitors of Leishmania CYP5122A1 warrant investigation as chemical probes and that compounds displaying activity against CYP5122A1 or as dual inhibitors of CYP51 and CYP5122A1 are of interest for antileishmanial drug discovery efforts.

A screen of recombinant L. donovani CYP51 and CYP5122A1 enzymes for inhibition by small molecules identified Maybridge compounds HTS06234 (4a) and HTS05515 (18a) as hit compounds. Compounds 4a and 18a were then used as starting points for the synthesis of improved inhibitors displaying in vitro antileishmanial activity. Differences in the selectivity of the target compounds for CYP5122A1 compared to CYP51 were also observed. The synthesis, characterization, and biological activity of these novel Leishmania CYP inhibitors is summarized here.

### Results and Discussion

Synthesis of analogs of compound 4a. Commercial hit compound 4a was resynthesized by reaction of commercially available 1-(pyridin-4-ylmethyl)piperazine (1) with 1-isocyanato-3,5-dimethylbenzene (3a) as shown in Scheme 1. Likewise, reaction between 1 and aryl isocyanates 3b-v yielded target compounds 4b-4v. Many of the aryl isocyanates used for the preparation of the target compounds were commercially available. Aryl isocyanates that could not be purchased were prepared from the corresponding arylamine compounds 2f-h, 21 via reaction with triphosgene.²²

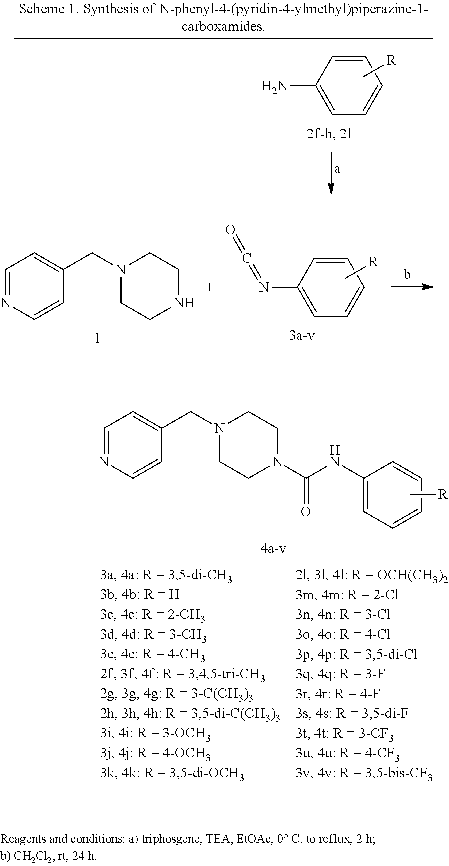

Synthesis of analogs of compound 18a. To prepare target compounds inspired by hit compound 18a, it was envisioned that reaction between 1-arylalkylpiperazines and 1-(benzyloxy)-4-isocyanatophenyl intermediates would provide the desired target molecules in an approach like that shown in Scheme 1. The synthesis of the 1-arylalkylpiperazines that could not be purchased is provided in Scheme 2. Reaction between 4-vinylpyridine (5) and Cbz-protected piperazine 6 in acetic acid/ethanol followed by palladium-catalyzed removal of the Cbz group afforded 1-(2-(pyridin-4-yl)ethyl)piperazine (7). 1-2-(pyridin-3-yl)ethyl)piperazine (10) was synthesized in two steps starting from 2-(pyridin-3-yl)ethan-1-ol (8). Reaction with tosyl chloride in the presence of triethylamine in DCM at room temperature yielded 2-(pyridin-3-yl)ethyl 4-methylbenzenesulfonate (9). Replacement of the tosyl group with piperazine in the presence of potassium carbonate in THF at reflux afforded intermediate 10. 1-Phenethyl piperazines 12a-j and 1-(2-(1H-pyrazol-1-yl)ethyl)piperazine (12k) were synthesized through reaction of the corresponding 2-bromoethylaryl precursors 11a-k with piperazine in the presence of potassium carbonate in THF at reflux. Other 1-arylalkylpiperazines used to synthesize target compounds 18c-ac were obtained commercially.

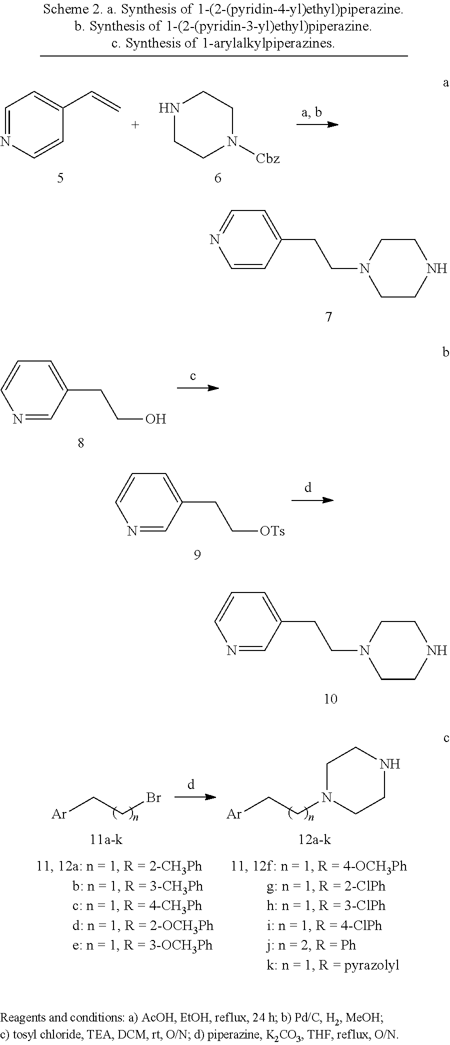

1-(Benzyloxy)-4-isocyanatophenyl intermediates needed to prepare the target PG-7C compounds were synthesized as outlined in Scheme 3. Reaction between p-nitrophenol (13) and benzyl bromides 14α-e yielded 1-(benzyloxy)-4-nitrobenzenes 15a-e, which were reduced to the corresponding 4-(benzyloxy)anilines 16a-e in the presence of zinc and ammonium chloride in methanol. Isocyanates 17a-e were derived from anilines 16a-e by reaction with triphosgene in the presence of triethylamine.

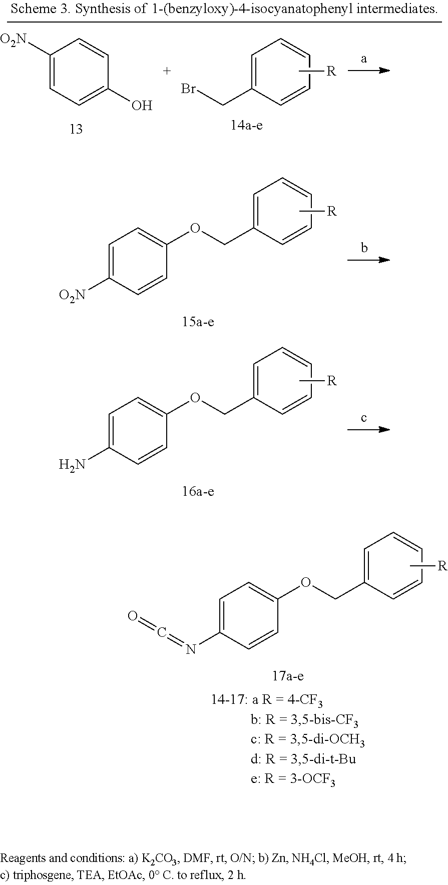

Scheme 4 shows the synthesis of target compound 18b. Isocyanate 17a was first converted to the corresponding piperazine derivative 17f by reaction of 17a with Boc-protected piperazine followed by deprotection and basic workup. Piperazine 17f then reacted with isonicotinaldehyde under reductive amination conditions to provide 18b.

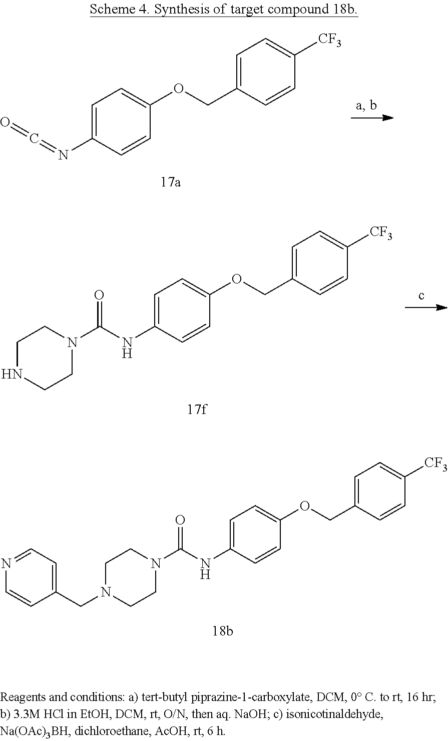 [SMILES: O=C=Nc1ccc(OCc2ccc(C(F)(F)F)cc2)cc1 ; O=C(Nc1ccc(OCc2ccc(C(F)(F)F)cc2)cc1)N1CCNCC1 ; O=C(Nc1ccc(OCc2ccc(C(F)(F)F)cc2)cc1)N1CCN(Cc2ccncc2)CC1]

Target compounds 18c-ac were synthesized by reaction of isocyanates 17a-e with 1-arylalkylpiperazines in DCM at room temperature as shown in Scheme 5. The original hit compound 18a was not resynthesized.

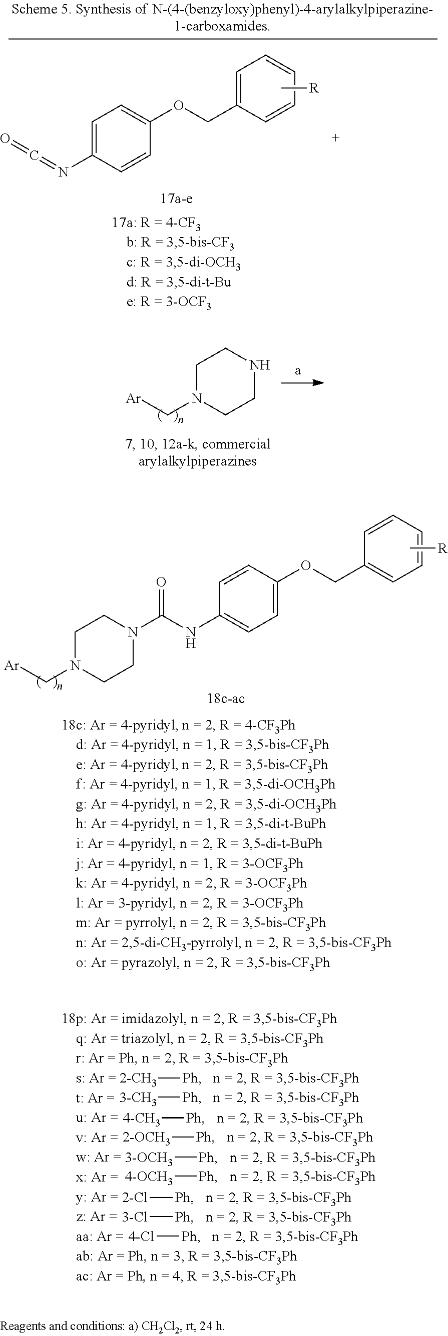

Evaluation of analogs of compound 4a. All the N-phenyl-4-(pyridin-4-ylmethyl)-piperazine-1-carboxamide target molecules based on hit compound 4a were inhibitors of recombinant Leishmania donovani CYP51, with IC₅₀ values ranging from 0.57 to 85 μM (see Table 1). The most potent inhibitors of L. donovani CYP51 possess substituents at the 3-position of the terminal phenyl (“C”) ring. For example, for the molecules substituted with a single methyl group, the 3-methyl derivative 4d was 7.1-fold more potent than the 2-methyl derivative 4c and 2.3-fold more potent than the 4-methyl derivative 4e. Similarly, for the molecules possessing a single chlorine substitution on the C ring, the 3-Cl analog 4n was 4.3-fold more potent than the 2-Cl congener 4m and 5.7-fold more potent than the 4-Cl derivative 4o. While the 2-substituted derivative was not prepared in the methoxy, fluoro, or trifluoromethyl series, the 3-substituted analog was more potent against CYP51 than the 4-substituted derivative in each case. When the C ring contained the same substituent at both the 3 and 5 positions, potency was similar to the 3-substituted derivative in the methyl series (compare 4a with 4d), higher potency was observed for the monosubstituted congener when the substituent was t-butyl (compare 4g with 4h), and higher potency was noted for the disubstituted derivative when the substituents were methoxy (compare 4i with 4k), chlorine (compare 4n with 4p), fluorine (compare 4q with 4s), or trifluoromethyl (compare 4t with 4v). Given that the most potent derivatives possessed 3,5-di-Cl (4p) and 3,5-bis-CF₃ (4v) substituents on the C ring, electron withdrawing, lipophilic groups at these positions appear to be favored for CYP51 activity in this series. In terms of activity against L. donovani CYP5122A1, these 3-ring analogs show lower potency overall, with IC₅₀ values ranging from 9.6 to >100 μM. In contrast to CYP51, where 20 of the 22 analogs exhibited IC₅₀ values≤30 μM, only six of these target compounds possessed IC₅₀ values≤30 μM. All but two of these compounds with the highest potency against CYP5122A1 possess a 3,5-disubstituted phenyl ring, with the dimethyl (4a), di-t-butyl (4h) and bis-trifluoromethyl (4v) derivatives exhibiting the greatest potency (all with IC₅₀ values ˜10 μM). Given the potency of both the di-t-butyl (4h) and di-trifluoromethyl (4v) derivatives against CYP5122A1, lipophilicity may be more important that electron-withdrawing character for C ring substituents to enhance inhibition of this enzyme.

Thirteen of the twenty-two target compounds display negligible effects on the proliferation of L. donovani LV82 promastigotes (EC₅₀>100 μM). While some of these compounds show activity against CYP51, none are potent inhibitors of CYP5122A1 (for the thirteen compounds with negligible influence on L. donovani proliferation, the lowest IC₅₀ against CYP5122A1 is 86 μM). Four compounds possess EC₅₀ values of <30 μM against L. donovani promastigotes. These inhibitors of L. donovani proliferation also display IC₅₀ values against CYP5122A1≤30 μM and IC₅₀ values against CYP51<30 μM. Interestingly, the di-t-butyl compound (4h) displays balanced inhibition of CYP51 (IC₅₀=7.6 μM) and CYP5122A1 (IC₅₀=9.6 μM) and inhibits promastigote proliferation (EC₅₀=21 μM), while compounds 4q and 4s, which show similar IC₅₀ values to 4h against CYP51 but IC₅₀ values>100 μM against CYP5122A1, have little effect on L. donovani proliferation (EC₅₀>100 μM). It appears that inhibition of both CYP51 and CYP5122A1 is required for these target compounds to block the proliferation of L. donovani promastigotes.

Compounds 4g and 4v, the most potent molecules against L. donovani promastigote proliferation (EC₅₀ values of approximately 10 μM), were evaluated for their effect on J774 murine macrophages. Compound 4g displayed over 5-fold selectivity for promastigotes compared to J774 cells, while the selectivity index for compound 4v was less than 2-fold.

TABLE 1
Activity of Target Compounds 4a-v Against L. donovani CYP51, L. donovani
CYP5122A1, and L. donovani LV82 Promastigotes

| | |
|---|---|
| | 4a-v |
| 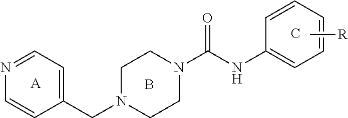 | |

| | IC₅₀ vs CYP51- | IC₅₀ vs CYP5122A1- | Selectivity Index | EC₅₀ vs | EC₅₀ vs J774 |
|---|---|---|---|---|---|
| Compound | 32c (μM)^{a} | 61c (μM)ª | (IC₅₀_32c/IC₅₀_61c) | LV82 (μM)^{b} | (μM)^{b} |
| 4a | 8.7 ± 1.0 | 10 ± 1 | 0.90 | >50 | ND^{c} |
| 4b | 28 ± 1 | >100 | <0.28 | >100 | ND |
| 4c | 85 ± 7 | >100 | <0.85 | >100 | ND |
| 4d | 12 ± 1 | >100 | <0.12 | >100 | ND |
| 4e | 28 ± 1 | >100 | <0.28 | >100 | ND |
| 4f | 7.1 ± 0.6 | 45 ± 4 | 0.16 | >>50 | ND |
| 4g | 4.4 ± 0.3 | 22 ± 1 | 0.20 | 10 | >50 |
| 4h | 7.6 ± 0.1 | 9.6 ± 2 | 0.80 | 21 | ND |
| | 16 ± 1 | >100 | <0.16 | >100 | ND |
| | 42 ± 2 | >100 | <0.42 | >100 | ND |
| | 5.3 ± 2 | >100 | <0.05 | >100 | ND |
| 4l | 14 ± 2 | 38 ± 1 | 0.38 | >50 | ND |
| 4m | 13 ± 1 | >100 | <0.13 | >100 | ND |
| 4n | 3.0 ± 0.1 | 93 ± 9 | 0.03 | >100 | ND |
| 4o | 17 ± 1 | 86 ± 8 | 0.20 | >100 | ND |
| 4p | 0.57 ± 0.01 | 30 ± 2 | 0.02 | 27 | ND |
| 4q | 9.5 ± 0.1 | >100 | <0.09 | >100 | ND |
| 4r | 29 ± 2 | >100 | <0.29 | >100 | ND |
| 4s | 3.6 ± 0.0 | >100 | <0.04 | >100 | ND |
| 4t | 3.3 ± 0.3 | 41 ± 4 | 0.08 | 48 | ND |
| 4u | 4.7 ± 0.4 | 28 ± 6 | 0.17 | >50 | ND |
| 4v | 0.77 ± 0.02 | 9.7 ± 0.8 | 0.08 | 13 | 23 |

^{a}Mean ± standard deviation (n = 3)
^{b}Determined by plotting the results of n ≥ 3 independent experiments on the same graph. In these experiments the standard compound amphotericin B displayed an EC₅₀ of 0.084 ± 0.022 μM against LV82 (mean ± std deviation, n = 23) and the standard compound podopbyllotoxin displayed an EC₅₀ of 0.013 ± 0.001 μM against J774 (mean ± std deviation, n = 3).
^{c}ND: not determined

Evaluation of analogs of compound 18a. As in the above series of compounds possessing three rings, the N-(4-(benzyloxy)phenyl)-4-(2-(pyridin-4-yl)alkyl)piperazine-1-carboxamide target compounds 18b-k based on high throughput screening hit 18a were all inhibitors of recombinant Leishmania donovani CYP51, but with IC₅₀ values ranging from 0.16 to 1.2 μM (Table 2). Thus, the addition of the benzyloxy substituent at the 4-position of the C ring imparted strong CYP51 inhibitory activity in a narrower range for these derivatives compared to the three ring target compounds, although the selection of D ring substituents may have also influenced these results. When the linker length between the A and B rings was two carbons, addition of 4-trifluoromethyl (18c), 3,5-bis-trifluoromethyl (18e), 3,5-di-t-butyl (18i), or 3-trifluoromethoxy (18k) groups to the D ring increased the potency against CYP51 by 1.9-2.8 fold compared to hit compound 18a, while addition of 3,5-dimethoxy substitutions (18g) decreased activity by 1.8-fold. The presence of a two-carbon linker between the A and B rings had a minor effect on CYP51 inhibition, increasing potency by roughly twofold in the 4-trifluoromethyl, 3,5-di-t-butyl, and 3-trifluoromethoxy series but decreasing potency by 1.9-fold in the 3,5-bis-trifluoromethyl series. Addition of the benzyloxy substituent at the 4-position of the C ring also imparted potent inhibitory activity against CYP5122A1. IC₅₀ values for inhibition of this enzyme were 0.98-10 μM for the target compounds. Again comparing target compounds with a two carbon linker between the A and B rings with the hit compound 18a, addition of the 3,5-bis-trifluoromethyl (18e) and the 3,5-di-t-butyl (18i) groups to the D ring increased inhibitory potency against CYP5122A1 by 3.4- and 2.2-fold, respectively, while addition of the 4-trifluoromethyl (18c) and the 3-trifluoromethoxy (18k) groups to the D ring had little effect on CYP5122A1 potency. Addition of 3,5-dimethoxy (18g) groups decreased potency against CYP5122A1 by 3.0-fold. The presence of a one- or two-carbon linker between the A and B rings had little effect on CYP5122A1 inhibition. In contrast, inhibition of promastigote proliferation was strongly influenced by the linker length between the A and B rings, with compounds possessing an ethylene linker being 2.2- to 6.6-fold more potent inhibitors of L. donovani promastigote growth than target compounds possessing a methylene linker. The most potent inhibitors of parasite proliferation were the compounds possessing the 3,5-bis-trifluoromethyl (18e), 3-trifluoromethoxy (18k), and 3,5-di-1-butyl (18i) groups, exhibiting EC₅₀ values of 1.4, 2.7, and 2.7 μM, respectively. While the relationship between CYP inhibition and growth inhibition is not clear, aside from hit compound 18a, the target compounds in Table 2 all display IC₅₀ values<2 μM against CYP51, IC₅₀ values C₁₀ μM against CYP5122A1, and EC₅₀ values<10 μM against L. donovani proliferation. Given these results and the superior growth inhibition for the target compounds possessing two-carbon linkers compared to one-carbon linkers, we speculate that differences in uptake/diffusion of the compounds into the parasite and modulation of other aspects of promastigote metabolism may also play a role in determining their antiparasitic potency. Since all the target compounds showed inhibition of promastigote growth with EC₅₀ values≤1 μM, the effect of compounds 18b-k on the proliferation of J774 macrophages was examined. While most of these compounds were not selective for parasites (EC (o J774/0o LV82<5), compounds 18e and 18i displayed tenfold and 5.9-fold selectivity for promastigotes compared to the mammalian macrophage cell line.

TABLE 2
Activity of N-(4-(benzyloxy)phenyl)-4-(2-(pyridin-4-yl)alkyl)piperazine-1-
carboxamides Against CYP51, CYP5122A1, and L. donovani LV82 Promastigotes

| | |
|---|---|
| | 18a-k |
| 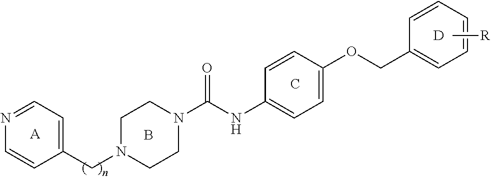 | |

| | | IC₅₀ vs | Selectivity Index | EC₅₀ vs | EC₅₀ vs |
|---|---|---|---|---|---|
| | IC₅₀ vs CYP51-32c | CYP5122A1- | (IC₅₀_32c/ | LV82 | J774 |
| Compound | (μM)^{a} | 61c (μM)^{a} | IC₅₀_61c) | (μM)^{b} | (μM)^{b} |
| 18a | 0.65 ± 0.05 | 3.3 ± 0.3 | 0.19 | >100 | ND^{c} |
| 18b | 0.77 ± 0.07 | 3.2 ± 0.3 | 0.24 | 11 | 4.3 |
| 18c | 0.35 ± 0.01 | 3.5 ± 0.2 | 0.10 | 5.1 | 2.3 |
| 18d | 0.16 ± 0.01 | 1.1 ± 0.1 | 0.14 | 9.3 | 15 |
| 18e | 0.30 ± 0.01 | 0.98 ± 0.01 | 0.30 | 1.4 | 14 |
| 18f | | | | | |
| 18g | 1.2 ± 0.0 | 10 ± 1 | 0.12 | 8.1 | 7.8 |
| 18h | 0.59 ± 0.05 | 1.6 ± 0.2 | 0.37 | 7.9 | 22 |
| 18i | 0.29 ± 0.04 | 1.5 ± 0.3 | 0.19 | 2.7 | 16 |
| 18j | 0.52 ± 0.04 | 2.8 ± 0.2 | 0.18 | 9.3 | 15 |
| 18k | 0.23 ± 0.02 | 2.9 ± 0.3 | 0.08 | 2.7 | 4.3 |

^{a}Mean # standard deviation (n = 3)
^{b}Determined by plotting the results of n ≥ 3 independent experiments on the same graph. In these experiments the standard compound amphotericin B displayed an EC₅₀ of 0.089 ± 0.025 μM against LV82 (mean ± std deviation, n = 15) and the standard compound podophyllotoxin displayed an EC₅₀ of 0.012 ± 0.001 μM against J774 (mean ± std deviation, n = 10).
^{C}ND: not determined

A series of analogs was also prepared based on 18e, the compound shown in Table 2 with the greatest potency against L. donovani promastigote proliferation. In this series of analogs, the 4-pyridyl ring present at position A in 18e was replaced by either a 3-pyridyl ring (compound 18l), a five-membered nitrogen-containing heterocycle (compounds 18m-q) or an unsubstituted or substituted phenyl ring (compounds 18r-ac). Interestingly, selectivity for CYP5122A1 compared to CYP51 is observed with compounds 18m-q, with imidazole 18p exhibiting the greatest inhibitory potency of any of the target compounds against CYP5122A1 (IC₅₀=0.20 μM). The potencies of pyrrole 18m and 1,2,4-triazole 18q against CYP5122A1 are 4.7-fold and 6.0-fold less than 18p, respectively, while the phenyl congener that also possesses a two-carbon linker (18r) is 37-fold less potent than imidazole 18p against CYP5122A1. While imidazole 18p remains a sub-micromolar CYP51 inhibitor (IC₅₀=0.77 μM), its selectivity for CYP5122A1 is 3.8-fold. Lower potency against CYP51 is seen with triazole 18q, pyrroles 18m and 18n, and phenyl derivative 18r, with the latter possessing negligible activity against CYP51 (IC₅₀>100 μM) and a selectivity index of >13 for CYP5122A1 compared to CYP51. Pyrroles 18m and 18n display selectivity indexes of 25 and 20 for CYP5122A1 compared to CYP51, respectively. The contribution of CYP inhibition toward parasite growth inhibition is not clear; 18e is a submicromolar inhibitor of both CYP51 and CYP5122A1 and inhibits L. donovani promastigote proliferation with an EC₅₀=1.4 μM, while 18p is a submicromolar inhibitor of both CYP51 and CYP5122A1 (albeit with a selectivity for CYP5122A1 over CYP51) and inhibits L. donovani promastigote proliferation with an EC₅₀=31 μM. Despite the potency of imidazole 18p against both CYP5122A1 and CYP51, pyrrole 18m is 2.8-fold more potent than 18p as an inhibitor of L. donovani promastigote proliferation regardless of its diminished effect on both CYP5122A1 and CYP51. As mentioned earlier, parasite uptake may influence growth inhibitory potency for this series, or other mechanisms of action may also be operative. Nevertheless, for the molecules reported here, activity against CYP5122A1 is necessary (although not sufficient) for antileishmanial activity.

TABLE 3
Activity of Benzyloxyphenyl-arylethylpiperazine-1-carboxamide Compounds
Against CYP51, CYP5122A1, and L. donovani LV82 Promastigotes

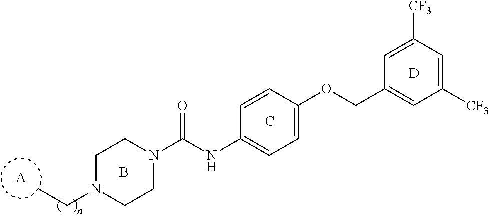

| | IC₅₀ vs | IC₅₀ vs | Selectivity Index | | |
|---|---|---|---|---|---|
| | CYP51-32c | CYP5122A1-61c | (IC₅₀_32c/ | EC₅₀ vs LV82 (μM)^{b} | EC₅₀ vs |
| Compound | (μM)^{a} | (μM)^{a} | IC₅₀_61c) | or [% inhibition @ 50 μM] | J774 (μM)^{b} |
| 18e | 0.30 ± 0.01 | 10.98 ± 0.01 | 0.30 | 1.4 | 14 |
| 18l | | | | | |
| 18m | 23 ± 3 | 0.93 ± 0.05 | 25 | 11 | 2.9 |
| 18n | 36 ± 4 | 1.8 ± 0.2 | 20 | 12 | 5.9 |
| 18o | 150 | 1.6 ± 0.1 | 9.4 | 30 | 11 |
| 18p | 0.77 ± 0.06 | 0.20 ± 0.02 | 3.8 | 31 | 19 |
| 18q | 3.3 ± 0.2 | 1.2 ± 0.2 | 2.7 | >50 | >50 |
| 18r | >100 | 7.4 ± 1.0 | >13 | >12.5 | 11 |
| 18s | | 5.1 ± 0.5 | — | 15 | 6.2 |
| 18t | | 5.6 ± 1.2 | — | 16 | 5.4 |
| 18u | 4.7 ± 1.4 | 5.4 ± 1.3 | 0.87 | 12 | 6.3 |
| 18v | | 4.1 ± 0.4 | — | 14 | 6.5 |
| 18w | 1.4 ± 0.1 | 3.4 ± 0.7 | 0.41 | 12 | 6.3 |
| 18x | | | — | | 8.3 |
| 18y | | 7.6 ± 1.0 | — | 14 | 6.6 |
| 18z | | 3.7 ± 0.2 | — | 15 | 6.2 |
| 18aa | | 5.0 ± 0.6 | — | 17 | 6.3 |
| 18ab | | 3.3 ± 1.3 | — | >50 | 4.0 |
| 18ac | 13 ± 2 | 18 ± 4 | 0.72 | 9.7 | 3.2 |

^{a}Mean ± standard deviation (n = 3)
^{b}Determined by plotting the results of n ≥ 3 independent experiments on the same graph. In these experiments the standard compound amphotericin B displayed an EC₅₀ of 0.097 ± 0.024 μM against LV82 (mean ± std deviation, n = 32) and the standard compound podophyllotoxin displayed an EC₅₀ of 0.013 ± 0.001 μM against J774 (mean ± std deviation, n = 15).

Evaluation in an infected macrophage model. Four of the compounds described above were selected for evaluation in an L. donovani infected macrophage model. These compounds were the two most active derivatives of original hit compound 4a (compounds 4g and 4v) along with the two most active analogs of screening hit 18a (compounds 18e and 18i). In addition to their potency in the promastigote assay, three of these four compounds also displayed modest selectivity for L. donovani compared to J774 macrophages. An image-based assay employing murine peritoneal macrophages as host cells and DAPI as an indicator that was based on assays reported previously in the literature²³⁻²⁴ was used to evaluate these compounds. Compounds 4g and 4v showed little effect on intracellular parasites at concentrations that were non-toxic to parasites. While compounds 18e and 18i exhibited toxicity to host cells at 50 μM concentrations, host cell toxicity was lower at concentrations of 25 μM and below, and these compounds were effective inhibitors of intracellular L. donovani. The EC₅₀ values of these compounds against intracellular L. donovani were 4.0 μM (n=3 determinations) and 1.6 μM (n=3 determinations), respectively.

Conclusions. Screening for activity against CYP51 and CYP5122A1 identified 4-pyridyl compounds 4a and 18a as potential hits. Gunatilleke et al.¹⁴ also identified 4-pyridyl-containing molecules as binders to T. cruzi CYP51, with two of these compounds being reported as an inhibitor of intracellular T. cruzi with an EC₅₀ value of <1 μM. Given the activity observed for the 4-pyridyl compounds reported here along with imidazole 14p and triazole 14q against both CYP51 and CYP5122A1, there is clearly overlap in the type of ligands accepted by both Leishmania CYP450 enzymes in addition to overlap among inhibitors of CYP51 from different organisms. N-phenyl-4-(pyridin-4-ylmethyl)piperazine-1-carboxamides based on 4a were generally strong inhibitors of CYP51 but were less potent against CYP5122A1 and typically displayed weak inhibition of L. donovani promastigote growth. N-(4-(benzyloxy)phenyl)-4-(2-(pyridin-4-yl)alkyl)piperazine-1-carboxamides based on 18a were stronger inhibitors of both CYP5122A1 and L. donovani promastigote proliferation but also remained selective for inhibition of CYP51. Two compounds in this series, 18e and 18i, showed selectivity for L. donovani promastigotes compared to J774 macrophages and were effective against intracellular L. donovani with EC₅₀ values in the low micromolar range. When a five membered nitrogen containing heterocycle was exchanged for the 4-pyridyl ring (the benzyloxyphenyl-arylethylpiperazine-1-carboxamide series), selectivity for CYP5122A1 over CYP51 was observed, but selective potency against L. donovani promastigotes was lost. Imidazole derivative 18p showed approximately fourfold selectivity for CYP5122A1 over CYP51 and inhibited both enzymes with IC₅₀ values≤1 μM, although the compound lacked potency against L. donovani promastigotes. Structure-based drug design efforts could aid in the development of more effective inhibitors of both Leishmania CYP51 and CYP5122A1.

This example builds on recent findings indicating that inhibition of both CYP51 and CYP5122A1 is necessary, but not sufficient, for antileishmanial activity.¹⁹ Based on their activity in the intracellular L. donovani assay, 18e and 18i are of interest as a new class of antileishmanial compounds that interfere with two individual steps in Leishmania sterol biosynthesis. Further analog synthesis and biological evaluation will be required to determine the value of dual inhibitors of CYP51 and CYP5122A1 as new antileishmanial drug candidates and the utility of these molecules as probes to shed further light on sterol biosynthesis in Leishmania and related parasites.

### Experimental Section

General Chemistry Methods. Reactions were monitored by TLC silica gel aluminum sheets (Sigma-Aldrich) and compounds were purified by Combi Flash chromatography using Teledyne ISCO CombiFlash (include version) using commercial grade ethyl acetate, hexanes, dichloromethane and methanol. Reagents were purchased from Sigma-Aldrich, Oakwood chemicals, Fisher Scientific, AABlocks, AmBeed, or Combi-Blocks and were used without further purification. NMR spectra were recorded on Bruker AV300 or DRX400 or Avance III HD Ascend 700 MHz spectrometers at 298 K unless noted and were calibrated using the residual solvent peak (CDCl₃: δ 7.26 ppm for ¹H NMR, 77.16 ppm for ¹³C NMR; DMSO-d₆: δ 2.50 ppm for ¹H NMR, 39.50 ppm for ¹³C NMR).²⁵ Compounds with peaks overlapping the residual solvent peak in ¹H NMR spectra were verified by obtaining an additional spectrum using a different solvent. Coupling for symmetrical ortho-coupled aromatic systems are reported, realizing that spectra are not first order. High resolution mass spectra (HRMS) were obtained using a Thermo Q-Exactive Orbitrap with Vanquish-H UHPLC. Elemental analysis was conducted by Atlantic Microlab, Inc, Norcross, GA.

General method for synthesis of 4a-v. To a stirred solution of substituted phenyl isocyanate (3a-v, 88-337 mg, 0.55-1.48 mmol, 1.0 equiv.) in DCM (10 mL) at room temperature was added 1-(pyridine-4-ylmethyl)piperazine (1, 143-438 mg, 0.81-2.47 mmol, 1.1-1.9 equiv.). The reaction mixture was stirred at room temperature overnight. After the reaction was complete as assessed by TLC analysis, the solvent was removed in vacuo and the crude product which was purified using CombiFlash chromatography eluting with MeOH/DCM to provide 4a-v as a oily liquid or white powder (139-602 mg, yield 32-99/6). Final products were recrystallized from ethyl acetate and hexanes to afford high purity compounds.

### N-(3,5-dimethylphenyl)-4-(pyridin-4-ylmethyl)piperazine-1-carboxamide (4a)

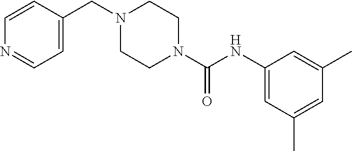 [SMILES: Cc1cc(C)cc(NC(=O)N2CCN(Cc3ccncc3)CC2)c1]

White powder, 487 mg, yield 74% starting from 298 mg of 3a (2.02 mmol); mp 141-142° C.; ¹H NMR (400 MHz, CDCl₃) δ 2.26 (s, 6H), 2.46 (t, J=5.0 Hz, 4H), 3.49 (t, J=5.0 Hz, 4H), 3.52 (s, 2H), 6.42 (br s, 1H), 6.67 (s, 1H), 6.97 (s, 2H), 7.26-7.29 (m, 2H, slightly overlapped with solvent peak), 8.53-8.57 (m, 2H). ¹³C NMR (100 MHz, CDCl₃) δ 21.5, 44.2, 52.9, 61.7, 117.9, 123.9, 125.0, 138.6, 138.9, 147.3, 150.0, 155.2. HRMS: m/z [M+H]⁺ calcd for C₁₉H₂₅N₄O, 325.20229; found, 325.20179; Elemental Analysis Calcd for C₁₉H₂₄N₄O: C, 70.34; H, 7.46; N, 17.27. Found: C, 70.32; H, 7.56; N, 17.27.

### N-phenyl-4-(pyridin-4-ylmethyl)piperazine-1-carboxamide (4b)

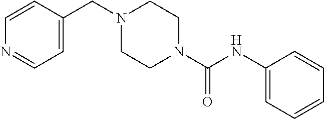 [SMILES: O=C(Nc1ccccc1)N1CCN(Cc2ccncc2)CC1]

White crystalline powder, 367 mg, yield 80% starting from 183 mg of 3b (1.54 mmol); mp 160-161° C.; ¹H NMR (400 MHz, DMSO-d₆) δ 2.37-2.43 (m, 4H), 3.43-3.50 (m, 4H), 3.55 (s, 2H), 6.89-6.96 (m, 1H), 7.18-7.25 (m, 2H), 7.32-7.37 (m, 2H), 7.41-7.47 (m, 2H), 8.48 (s, 1H), 8.50-8.54 (m, 2H). ¹³C NMR (100 MHz, CDCl₃) δ 44.2, 52.9, 61.7, 120.2, 123.2, 123.9, 128.9, 139.1, 147.3, 150.0, 155.2. HRMS: m/z [M+H]⁺ calcd for C₁₇H₂₁N₄O, 297.17099; found, 297.17052; Elemental Analysis Calcd for C₁₇H₂₀N₄O: C, 68.90; H, 6.80; N, 18.90. Found: C, 68.87; H, 6.71; N, 18.86.

### 4-(Pyridin-4-ylmethyl)-N-(o-tolyl)piperazine-1-carboxamide (4c)

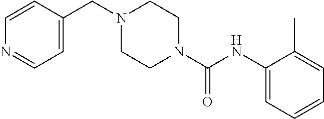 [SMILES: Cc1ccccc1NC(=O)N1CCN(Cc2ccncc2)CC1]

White powder, 279 mg, yield 76% starting from 158 mg of 3c (1.19 mmol), mp 167-168° C.; ¹H NMR (400 MHz, CDCl₃) δ 2.22 (s, 3H), 2.47 (t, J=5.1 Hz, 4H), 3.50 (t, J=5.1 Hz, 4H), 3.53 (s, 2H), 6.22 (br s, 1H), 7.00 (td, J=7.4, 1.1 Hz, 1H), 7.11-7.19 (m, 2H), 7.26-7.30 (m, 2H), 7.56 (br d, J=8.0 Hz, 1H), 8.51-8.58 (m, 2H). ¹³C NMR (100 MHz, CDCl₃) δ 17.9, 44.3, 52.9, 61.7, 123.2, 123.9, 124.3, 126.8, 129.2, 130.5, 137.0, 147.3, 150.0, 155.5. HRMS: m/z [M+H]⁺ calcd for C₁₈H₂₃N₄O, 311.18664; found, 311.18597; Elemental Analysis Calcd for C₁₈H₂₂N₄O: C, 69.65; H, 7.14; N, 18.05. Found: C, 69.59; H, 7.25; N, 18.08.

### 4-(Pyridin-4-ylmethyl)-N-(m-tolyl)piperazine-1-carboxamide (4d)

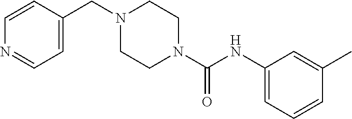 [SMILES: Cc1cccc(NC(=O)N2CCN(Cc3ccncc3)CC2)c1]

White crystalline powder, 218 mg, yield 54% starting from 175 mg of 3d (1.31 mmol); mp 153-154° C.; ¹H NMR (400 MHz, CDCl₃) δ 2.30 (s, 3H), 2.42-2.50 (m, 4H), 3.47-3.51 (m, 4H), 3.52 (s, 2H), 6.49 (br s, 1H), 6.84 (br d, J=7.2 Hz, 1H), 7.07-7.11 (m, 1H), 7.12-7.17 (m, 1H), 7.21 (br s, 1H), 7.26-7.30 (m, 2H, slight overlap with solvent peak), 8.53-8.57 (m, 2H). ¹³C NMR (100 MHz, CDCl₃) δ 21.6, 44.2, 52.9, 61.7, 117.2, 120.9, 123.9, 124.1, 128.8, 138.8, 139.0, 147.3, 150.0, 155.2. HRMS: m/z [M+H]⁺ calcd for C₁₈H₂₃N₄O, 311.18664; found, 311.18593; Elemental Analysis Calcd for C₁₈H₂₂N₄O: C, 69.65; H, 7.14; N, 18.05. Found: C, 69.61; H, 7.12; N, 17.95.

### 4-(Pyridin-4-ylmethyl)-N-(p-tolyl)piperazine-1-carboxamide (4e)

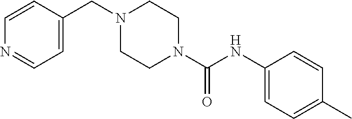 [SMILES: Cc1ccc(NC(=O)N2CCN(Cc3ccncc3)CC2)cc1]

White crystalline powder, 327 mg, yield 82% starting from 170 mg of 3e (1.28 mmol); mp 159-160° C.; ¹H NMR (400 MHz, CDCl₃) δ 2.28 (s, 3H), 2.45 (t, J=5.0 Hz, 4H), 3.49 (t, J=5.1 Hz, 4H), 3.52 (s, 2H), 6.49 (br s, 1H), 7.06 (d, 1=8.2 Hz, 2H), 7.21 (d, J=8.5 Hz, 2H), 7.26-7.29 (m, 2H, slight overlap with solvent peak), 8.53-8.57 (m, 2H). ¹³C NMR (100 MHz, CDCl₃) δ 20.9, 44.2, 52.9, 61.7, 120.4, 123.9, 129.5, 132.8, 136.5, 147.3, 150.0, 155.4. HRMS: m/z [M+H]⁺ calcd for C₁₈H₂₃N₄O, 311.18664; found, 311.18588; Elemental Analysis Calcd for C₁₈H₂₂N₄O: C, 69.65; H, 7.14; N, 18.05. Found: C, 69.63; H, 7.11; N, 17.96.

### 4-(pyridin-4-ylmethyl)-N-(3,4,5-trimethylphenyl)piperazine-1-carboxamide (4f)

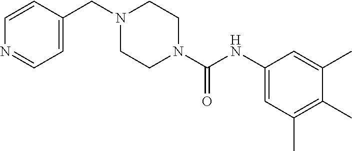 [SMILES: Cc1cc(NC(=O)N2CCN(Cc3ccncc3)CC2)cc(C)c1C]

White powder, 139 mg, yield 75% starting from 88 mg of 3f (0.55 mmol); ¹H NMR (400 MHz, CDCl₃) δ 2.09 (s, 3H), 2.22 (s, 6H), 2.45 (t, J=5.0 Hz, 4H), 3.48 (t, J=5.0 Hz, 4H), 3.52 (s, 2H), 6.36 (s, 1H), 6.99 (s, 2H), 7.26-7.29 (m, 2H, slight overlap with solvent peak), 8.53-8.56 (m, 2H). ¹³C NMR (100 MHz, CDCl₃) S 14.9, 20.8, 44.2, 52.9, 61.7, 119.7, 123.9, 130.1, 136.0, 137.0, 147.3, 150.0, 155.4. HRMS: m/z [M+H]⁺ calcd for C₂₀H₂₇N₄O, 339.21794; found, 339.21730; Elemental Analysis Calcd for C₂₀H₂₆N₄O: C, 70.98; H, 7.74; N, 16.55. Found: C, 71.02; H, 7.71; N, 16.45.

### N-(3-(tert-butyl)phenyl)-4-(pyridin-4-ylmethyl)piperazine-1-carboxamide (4g)

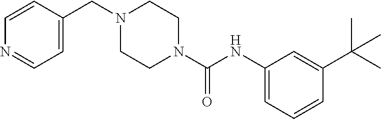 [SMILES: CC(C)(C)c1cccc(NC(=O)N2CCN(Cc3ccncc3)CC2)c1]

Off-white/pink powder, 140 mg, yield 66% starting from 106 mg of 3g (0.60 mmol); mp 125-127° C.; ¹H NMR (400 MHz, CDCl₃) δ 1.30 (s, 9H), 2.48 (t, J=5.1 Hz, 4H), 3.49-3.55 (m, 6H), 6.45 (br s, 1H), 7.06 (dt, J=7.1, 1.8 Hz, 1H), 7.15-7.24 (m, 2H), 7.27-7.30 (m, 2H), 7.34-7.37 (m, 1H), 8.53-8.58 (m, 2H). ¹³C NMR (100 MHz, CDCl₃) δ 31.4, 34.8, 44.2, 53.0, 61.7, 117.3, 117.4, 120.4, 123.9, 128.6, 138.8, 147.3, 150.0, 152.2, 155.3. HRMS: m/z [M+H]⁺ calcd for C₂₁H₂₉N₄O, 353.23359; found, 353.23152; Elemental Analysis Calcd for C₂₁H₂₈N₄O: C, 71.56; H, 8.01; N, 15.90. Found: C, 71.41; H, 8.02; N, 15.79.

### N-(3,5-di-tert-butylphenyl)-4-(pyridin-4-ylmethyl)piperazine-1-carboxamide (4h)

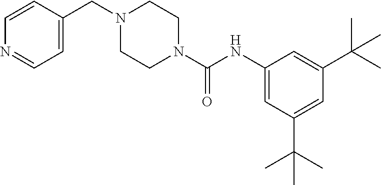 [SMILES: CC(C)(C)c1cc(NC(=O)N2CCN(Cc3ccncc3)CC2)cc(C(C)(C)C)c1]

White powder, 286 mg, yield 92% starting from 175 mg of 3h (0.76 mmol); ¹H NMR (400 MHz, CDCl₃) δ 1.30 (s, 18H), 2.49 (t, J=5.0 Hz, 4H), 3.51-3.55 (m, 6H), 6.39 (s, 1H), 7.11 (t, J=1.7 Hz, 1H), 7.20 (d, 2H, J=1.7 Hz), 7.27-7.30 (m, 2H), 8.54-8.57 (m, 2H). ¹³C NMR (100 MHz, CDCl₃) δ 31.6, 35.0, 44.3, 53.0, 61.8, 114.8, 117.6, 123.9, 138.3, 147.3, 150.0, 151.6, 155.4. HRMS: m/z [M+H]⁺ calcd for C₂₅H₃₇N₄O, 409.29619; found, 409.29560; Elemental Analysis Calcd for C₂₅H₃₆N₄O: C, 73.49; H, 8.88; N, 13.71. Found: C, 73.52; H, 8.90; N, 13.65.

### N-(3-methoxyphenyl)-4-(pyridin-4-ylmethyl)piperazine-1-carboxamide (4i)

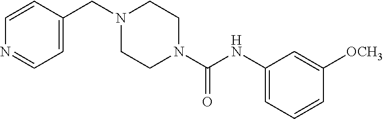 [SMILES: COc1cccc(NC(=O)N2CCN(Cc3ccncc3)CC2)c1]

Off-white powder, 365 mg, yield 76% starting from 220 mg of 3 (1.48 mmol); mp 127-129° C.; ¹H NMR (400 MHz, CDCl₃) δ 2.46 (t, J=5.0 Hz, 4H), 3.47-3.53 (m, 6H), 3.76 (s, 3H), 6.57 (ddd, J=8.3, 2.5, 0.7 Hz, 1H), 6.62 (s, 1H), 6.82 (ddd, J=8.0, 2.0, 0.8 Hz, 1H), 7.10-7.16 (m, 2H), 7.26-7.29 (m, 2H, overlapped with solvent peak), 8.53-8.56 (m, 2H). ¹³C NMR (100 MHz, CDCl₃) δ 44.2, 52.9, 55.4, 61.7, 105.6, 109.2, 112.1, 123.9, 129.6, 140.4, 147.3, 150.0, 155.0, 160.3. HRMS m/z [M+H]⁺ calcd for C₁₈H₂₃N₄O₂, 327.18155; found, 327.18088; Elemental Analysis Calcd for C₁₈H₂₂N₄O₂: C, 66.24; H, 6.79; N, 17.17. Found: C, 65.98; H, 6.69; N, 17.12.

### N-(4-methoxyphenyl)-4-(pyridin-4-ylmethyl)piperazine-1-carboxamide (4j)

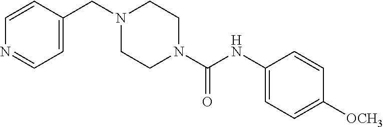 [SMILES: COc1ccc(NC(=O)N2CCN(Cc3ccncc3)CC2)cc1]

White crystalline powder, 392 mg, yield 90% starting from 199 mg of 3j (1.33 mmol); mp 173-174° C.; ¹H NMR (400 MHz, DMSO-d₆) δ 2.39 (t, J=4.9 Hz, 4H), 3.40-3.47 (m, 2H), 3.55 (s, 2H), 3.69 (s, 3H), 6.81 (d, J=9.1 Hz, 2H), 7.28-7.37 (m, 4H), 8.32 (br s, 1H), 8.50-8.54 (m, 2H). ¹³C NMR (100 MHz, CDCl₃) δ 44.1, 52.9, 55.6, 61.7, 114.1, 122.6, 123.9, 132.1, 147.3, 149.9, 155.7, 156.0. HRMS: m/z [M+H]⁺ calcd for C₁₈H₂₃N₄O₂, 327.18155; found, 327.18085; Elemental Analysis Calcd for C₁₈H₂₂N₄O₂: C, 66.24; H, 6.79; N, 17.17. Found: C, 66.10; H, 6.70; N, 17.05.

### N-(3,5-dimethoxyphenyl)-4-(pyridin-4-ylmethyl)piperazine-1-carboxamide (4k)

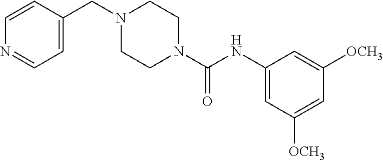 [SMILES: COc1cc(NC(=O)N2CCN(Cc3ccncc3)CC2)cc(OC)c1]

Off-white crystalline powder, 351 mg, yield 75% starting from 234 mg of 3k (1.31 mmol); mp 162-163° C.; ¹H NMR (400 MHz, DMSO-d₆) δ 2.39 (t, J=4.9 Hz, 4H), 3.41-3.47 (m, 4H), 3.55 (s, 2H), 3.68 (s, 6H), 6.08 (t, J=2.3 Hz, 1H), 6.75 (d, J=2.2 Hz, 2H), 7.31-7.36 (m, 2H), 8.43 (br s, 1H), 8.49-8.55 (m, 2H). ¹³C NMR (100 MHz, CDCl₃) δ 44.2, 52.9, 55.4, 61.7, 95.7, 98.1, 123.9, 141.1, 147.3, 150.0, 155.0, 161.1. HRMS: m/z [M+H]⁺ calcd for C₁₉H₂₅N₄O₃, 357.19212; found, 357.19050; Elemental Analysis Calcd for C₁₉H₂₄N₄O₃: C, 64.03; H, 6.79; N, 15.72. Found: C, 64.09; H, 6.71; N, 15.67.

### N-(3-isopropoxyphenyl)-4-(pyridin-4-ylmethyl)piperazine-1-carboxamide (4l)

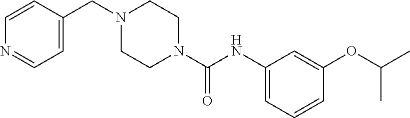 [SMILES: CC(C)Oc1cccc(NC(=O)N2CCN(Cc3ccncc3)CC2)c1]

Off-white/yellow powder, 190 mg, 90% yield starting from 106 mg of 31 (0.60 mmol); mp 134-136° C.; ¹H NMR (400 MHz, DMSO-d₆) δ 1.24 (d, J=6.0 Hz, 6H), 2.39 (t, J=4.9 Hz, 4H), 3.42-3.48 (m, 4H), 3.55 (s, 2H), 4.49 (septet, J=6.1 Hz, 1H), 6.44-6.49 (m, 1H), 6.95-7.00 (m, 1H), 7.08 (t, J=8.1 Hz, 1H), 7.12 (t, J=2.2 Hz, 1H), 7.29-7.37 (m, 2H), 8.43 (s, 1H), 8.49-8.54 (m, 2H). ¹³C NMR (100 MHz, CDCl₃) δ 22.2, 44.2, 52.9, 61.7, 70.0, 107.7, 111.1, 112.0, 123.9, 129.6, 140.4, 147.3, 150.0, 155.1, 158.6. HRMS: m/z [M+H]⁺ calcd for C₂₀H₂₇N₄O₂, 355.21285; found, 355.21053; Elemental Analysis Calcd for C₂₀H₂₆N₄O₂: C, 67.77; H, 7.39; N, 15.81. Found: C, 67.98; H, 7.36; N, 15.59.

### N-(2-chlorophenyl)-4-(pyridin-4-ylmethyl)piperazine-1-carboxamide (4m)

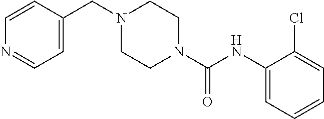 [SMILES: O=C(Nc1ccccc1Cl)N1CCN(Cc2ccncc2)CC1]

White powder, 139 mg, yield 32% starting from 204 mg of 3m (1.33 mmol); mp 127-128° C.; ¹H NMR (400 MHz, DMSO-d₆) δ 2.41 (t, J=4.9 Hz, 4H), 3.44-3.50 (m, 4H), 3.56 (s, 2H), 7.13 (td, J=7.7, 1.6 Hz, 1H), 7.27 (td, J=7.7, 1.5 Hz, 1H), 7.33-7.37 (m, 2H), 7.44 (dd, J=8.0, 1.4 Hz, 1H), 7.48 (dd, J=8.0, 1.6 Hz, 1H), 8.16 (br s, 1H), 8.49-8.55 (m, 2H). ¹³C NMR (100 MHz, CDCl₃) δ 44.2, 52.9, 61.7, 121.0, 122.4, 123.3, 123.9, 127.9, 128.9, 135.8, 147.1, 150.1, 154.2. HRMS: m/z [M+H]⁺ calcd for C₁₇H₂₀ClN₄O, 331.13202; found, 331.13129; Elemental Analysis Calcd for C₁₇H₁₉ClN₄O: C, 61.72; H, 5.79; N, 16.94. Found: C, 61.46; H, 5.80; N, 16.82.

### N-(3-chlorophenyl)-4-(pyridin-4-ylmethyl)piperazine-1-carboxamide (4n)

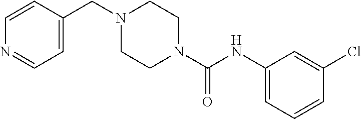 [SMILES: O=C(Nc1cccc(Cl)c1)N1CCN(Cc2ccncc2)CC1]

Off-white powder, 462 mg, yield 94% starting from 227 mg of 3n (1.48 mmol); mp 127-129° C.; ¹H NMR (400 MHz, CDCl₃) δ 2.41-2.47 (m, 4H), 3.47-3.53 (m, 6H), 6.80 (br s, 1H), 6.95-7.00 (m, 1H), 7.12-7.22 (m, 2H), 7.26-7.30 (m, 2H, overlapped with solvent peak), 7.41-7.44 (m, 1H), 8.52-8.57 (m, 2H). ¹³C NMR (100 MHz, CDCl₃) δ 44.2, 52.9, 61.7, 118.1, 120.1, 123.1, 123.9, 129.9, 134.5, 140.5, 147.3, 150.0, 154.8. HRMS: m/z [M+H]⁺ calcd for C₁₇H₂₀ClN₄O, 331.13202; found, 331.13117; Elemental Analysis Calcd for C₁₇H₁₉ClN₄O: C, 61.72; H, 5.79; N, 16.94. Found: C, 61.61; H, 5.77; N, 16.68.

### N-(4-chlorophenyl)-4-(pyridin-4-ylmethyl)piperazine-1-carboxamide (4o)

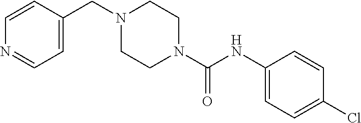 [SMILES: O=C(Nc1ccc(Cl)cc1)N1CCN(Cc2ccncc2)CC1]

White crystals, 308 mg, yield 82% starting from 174 mg of 3o (1.13 mmol); mp 184-185° C.; ¹H NMR (400 MHz, DMSO-d₆) δ 2.40 (t, J=5.0 Hz, 4H), 3.43-3.49 (m, 4H), 3.55 (s, 2H), 7.26 (d, J=9.0 Hz, 2H), 7.32-7.37 (m, 2H), 7.48 (d, J=9.0 Hz, 2H), 8.48-8.55 (m, 2H), 8.62 (br s, 1H). ¹³C NMR (100 MHz, CDCl₃) δ 44.2, 52.9, 61.7, 121.4, 123.9, 128.2, 128.9, 137.8, 147.3, 150.0, 155.0. HRMS: m/z [M+H]⁺ calcd for C₁₇H₂₀ClN₄O, 331.13202; found, 331.13140; Elemental Analysis Calcd for C₁₇H₁₉ClN₄O: C, 61.72; H, 5.79; N, 16.94. Found: C, 61.75; H, 5.71; N, 16.94.

### N-(3,5-dichlorophenyl)-4-(pyridin-4-ylmethyl)piperazine-1-carboxamide (4p)

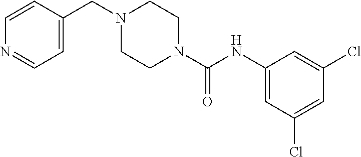 [SMILES: O=C(Nc1cc(Cl)cc(Cl)c1)N1CCN(Cc2ccncc2)CC1]

White powder, 365 mg, yield 85% starting from 220 mg of 3p (1.17 mmol); mp 183-184° C.; ¹H NMR (400 MHz, CDCl₃) δ 2.44 (t, J=5.0 Hz, 4H), 3.47-3.54 (m, 6H), 6.97 (t, J=1.8 Hz, 1H), 7.01 (br s, 1H), 7.26-7.30 (m, 4H, minimal overlap with solvent peak), 8.52-8.56 (m, 2H). ¹³C NMR (100 MHz, CDCl₃) δ 44.2, 52.8, 61.6, 118.2, 122.9, 124.0, 135.0, 141.3, 147.3, 150.0, 154.4. HRMS: m/z [M+H]⁺ calcd for C₁₇H₁₉Cl₂N₄O, 365.09304; found, 365.09229; Elemental Analysis Calcd for C₁₇H₁₉Cl₂N₄O: C, 55.90; H, 4.97; N, 15.34. Found: C, 55.72; H, 5.03; N, 15.22.

### N-(3-fluorophenyl)-4-(pyridin-4-ylmethyl)piperazine-1-carboxamide (4q)

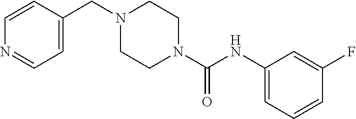 [SMILES: O=C(Nc1cccc(F)c1)N1CCN(Cc2ccncc2)CC1]

White crystalline powder, 293 mg, yield 82% starting from 155 mg of 3q (1.13 mmol); mp 157-158° C.; ¹H NMR (400 MHz, DMSO-d₆) δ 2.40 (t, J=4.9 Hz, 4H), 3.42-3.51 (m, 4H), 3.55 (s, 2H), 6.67-6.77 (m, 1H), 7.20-7.28 (m, 2H), 7.31-7.38 (m, 2H), 7.38-7.45 (m, 1H), 8.48-8.56 (m, 2H), 8.70 (s, 1H). ¹³C NMR (100 MHz, CDCl₃) δ 44.2, 52.9, 61.7, 107.3 (d, J=26.2 Hz), 109.8 (d, J=21.3 Hz), 115.1 (d, J=2.7 Hz), 123.9, 129.9 (d, 1=9.5 Hz), 140.9 (d, J=11.0 Hz), 147.2, 150.0, 154.7, 163.2 (d, J=244.1 Hz). HRMS: m/z [M+H]⁺ calcd for C₁₇H₂₀FN₄O, 315.16157, found, 315.16084; Elemental Analysis Calcd for C₁₇H₁₉FN₄O: C, 64.95; H, 6.09; N, 17.82. Found: C, 64.68; H, 6.08; N, 17.63.

### N-(4-fluorophenyl)-4-(pyridin-4-ylmethyl)piperazine-1-carboxamide (4r)

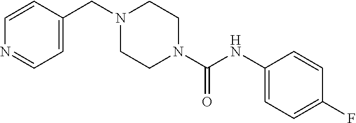 [SMILES: O=C(Nc1ccc(F)cc1)N1CCN(Cc2ccncc2)CC1]

White crystalline powder, 364 mg, yield 83% starting from 190 mg of 3r (1.39 mmol); mp 165-166° C.; ¹H NMR (400 MHz, DMSO-d₆) δ 2.39 (t, J=4.9 Hz, 4H), 3.42-3.49 (m, 4H), 3.55 (s, 2H), 7.02-7.09 (m, 2H), 7.30-7.37 (m, 2H), 7.40-7.48 (m, 2H), 8.50-8.54 (m, 3H). ¹³C NMR (100 MHz, CDCl₃) δ 44.1, 52.9, 61.7, 115.5 (d, J=22.4 Hz), 122.3 (d, J=7.8 Hz), 123.9, 135.1 (J=2.3 Hz), 147.3, 150.0, 155.4, 159.0 (d, J=244.1 Hz). HRMS: m/z [M+H]⁺ calcd for C₁₇H₂₀FN₄O, 315.16157; found, 315.16077; Elemental Analysis Calcd for C₁₇H₁₉FN₄O: C, 64.95; H, 6.09; N, 17.82. Found: C, 65.07; H, 6.05; N, 17.69.

### N-(3,5-difluorophenyl)-4-(pyridin-4-ylmethyl)piperazine-1-carboxamide (4s)

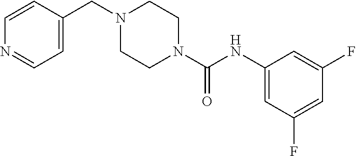 [SMILES: O=C(Nc1cc(F)cc(F)c1)N1CCN(Cc2ccncc2)CC1]

White powder, 602 mg, yield 99% starting from 283 mg of 3s (1.82 mmol); mp 168-169° C.; ¹H NMR (400 MHz, CDCl₃) S ¹H NMR (400 MHz, CDCl₃) δ 2.41-2.50 (m, 4H), 3.45-3.57 (m, 6H), 6.44 (tt, J=8.9, 2.3 Hz, 1H), 6.90-7.02 (m, 3H), 7.26-7.30 (m, 2H), 8.49-8.60 (m, 2H). ¹³C NMR (100 MHz, CDCl₃) δ 44.2, 52.9, 61.6, 98.1 (t, J=25.7 Hz), 102.6 (dd, J=20.7, 8.7 Hz), 124.0, 141.7 (t, J=13.5 Hz), 147.3, 150.0, 154.4, 163.3 (dd, J=245.4, 14.9 Hz). HRMS: m/z [M+H]⁺ calcd for C₁₇H₁₉F₂N₄O, 333.15214; found, 333.15147; Elemental Analysis Calcd for C₁₇H₁₈F₂N₄O: C, 61.44; H, 5.46; N, 16.86. Found: C, 61.48; H, 5.46; N, 16.81.

### 4-(Pyridin-4-ylmethyl)-N-(3-(trifluoromethyl)phenyl)piperazine-1-carboxamide (4t)

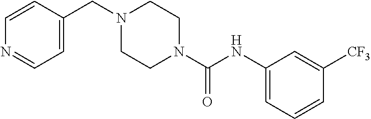 [SMILES: O=C(Nc1cccc(C(F)(F)F)c1)N1CCN(Cc2ccncc2)CC1]

White crystalline powder, 386 mg, yield 88% starting from 227 mg of 3t (1.21 mmol); mp 148-150° C.; ¹H NMR (400 MHz, DMSO-d₆) δ 2.41 (t, J=4.9 Hz, 4H), 3.45-3.52 (m, 4H), 3.56 (s, 2H), 7.25 (br d, J=7.7 Hz, 1H), 7.32-7.37 (m, 2H), 7.45 (t, J=8.0 Hz, 1H), 7.73 (br d, J=8.8 Hz, 1H), 7.91 (br s, 1H), 8.48-8.55 (m, 2H), 8.84 (br s, 1H). ¹³C NMR (100 MHz, CDCl₃) δ 44.2, 52.9, 61.7, 116.7 (q, J=4.0 Hz), 119.7 (q, J=3.8 Hz), 123.1-123.2 (m), 123.9, 124.1 (q, J=272.4 Hz), 129.4, 131.2 (q, J=32.2 Hz), 139.8, 147.3, 150.0, 154.8. HRMS: m/z [M+H]⁺ calcd for C₁₈H₂₀F₃N₄O, 365.15837; found, 365.15679; Elemental Analysis Calcd for C₁₈H₁₉F₃N₄O: C, 59.33; H, 5.26; N, 15.38. Found: C, 59.20; H, 5.39; N, 15.46.

### 4-(Pyridin-4-ylmethyl)-N-(4-(trifluoromethyl)phenyl)piperazine-1-carboxamide (4u)

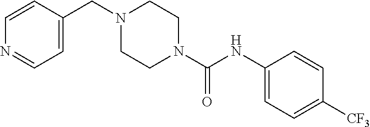 [SMILES: O=C(Nc1ccc(C(F)(F)F)cc1)N1CCN(Cc2ccncc2)CC1]

Off-white/orange powder, 218 mg, yield 70% starting from 161 mg of 3u (0.86 mmol); mp 121-123° C.; ¹H NMR (400 MHz, CDCl₃) S 2.46 (t, J=5.0 Hz, 4H), 3.48-3.57 (m, 6H), 6.92 (br s, 1H), 7.26-7.30 (m, 2H), 7.43-7.53 (m, 4H), 8.52-8.57 (m, 2H). ¹³C NMR (100 MHz, CDCl₃) δ 44.2, 52.9, 61.6, 119.3, 123.9, 124.4 (q, J=271.3 Hz), 124.8 (q, J=32.7 Hz), 126.2 (q, J=3.8 Hz), 142.5, 147.2, 150.0, 154.6. HRMS: m/z [M+H]⁺ calcd for C₁₈H₂₀F₃N₄O, 365.15837; found, 365.15692; Elemental Analysis Calcd for C₁₈H₁₉F₃N₄O: C, 59.33; H, 5.26; N, 15.38. Found: C, 59.11; H, 5.31; N, 15.42.

### N-(3,5-bis(trifluoromethyl)phenyl)-4-(pyridin-4-ylmethyl)piperazine-1-carboxamide (4v)

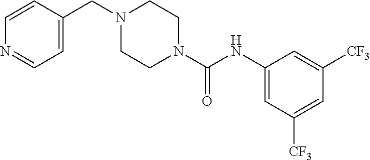 [SMILES: O=C(Nc1cc(C(F)(F)F)cc(C(F)(F)F)c1)N1CCN(Cc2ccncc2)CC1]

Light orange/off-white powder, 518 mg, yield 81% starting from 377 mg of 3v (1.48 mmol); mp 161-162° C.; ¹H NMR (400 MHz, DMSO-d₆) δ 2.42 (brt, J=4.9 Hz, 4H), 3.49-3.53 (m, 4H), 3.56 (s, 2H), 7.34-7.36 (m, 2H), 7.59 (s, 1H), 8.20 (s, 2H), 8.51-8.54 (m, 2H), 9.18 (s, 1H). ¹³C NMR (100 MHz, DMSO-d₆) δ 43.7, 52.4, 60.4, 113.9-114.1 (m), 118.5-118.7 (m), 123.4 (q, J_{C-F}=272.8 Hz), 123.7, 130.3 (q, J_{C-F}=32.5 Hz), 142.7, 147.1, 149.6, 154.1. HRMS: m/z [M+H]⁺ calcd for C₁₉H₁₉F₆N₄O, 433.14576; found, 433.14508; Elemental Analysis Calcd for C₁₉H₁₈F₆N₄O: C, 52.78; H, 4.20; N, 12.96. Found: C, 52.94; H, 4.15; N, 12.96.

### 4-(Pyridin-4-ylmethyl)-N-(4-((4-trifluoromethyl)benzyl)oxy)phenyl)piperazine-1-carboxamide (PS2-103, 18b)

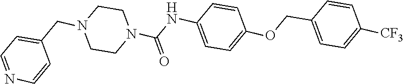 [SMILES: O=C(Nc1ccc(OCc2ccc(C(F)(F)F)cc2)cc1)N1CCN(Cc2ccncc2)CC1]

White solid, 160 mg, yield 45% from 80 mg of isonicotinaldehyde (0.75 mmol); mp 143-144° C.; ¹H NMR (400 MHz, CDCl₃) δ 2.46 (d, J=5.0 Hz, 4H), 3.46-3.51 (m, 4H), 3.52 (s, 2H), 5.07 (s, 2H), 6.46 (s, 1H), 6.87 (d, J=9.0 Hz, 2H), 7.22-7.30 (m, 4H, overlapped with residual solvent peak), 7.52 (d, J=8.1 Hz, 2H), 7.62 (d, J=8.2 Hz, 2H), 8.55 (d, J=5.8 Hz, 2H); ¹³C NMR (100 MHz, CDCl₃) δ 44.1, 52.9, 61.7, 69.5, 115.3, 122.4, 123.9, 124.2 (q, J_{C-F}=272.1 Hz), 125.6 (q, J_{C-F}=3.6 Hz), 127.5, 130.1 (q, J_{C-F}=32.5 Hz), 132.7, 141.3-141.4 (m), 147.3, 150.0, 154.7, 155.5; HRMS m/z [M+H]+ calcd for C₂₅H₂₆F₃N₄O₂, 471.2002; found, 471.1999; Anal. Calcd for C₂₅H₂₅F₃N₄O₂: C, 63.82; H, 5.36; N, 11.91; Found: C, 63.79; H, 5.31; N, 11.90.

General Procedure for synthesis of 18c-ac. To a stirred solution of isocyanates 17a-e (100-280 mg, 0.28-0.91 mmol, 1.0 equiv.) in DCM (10-20 mL) at room temperature was added the arylalkylpiperazine (1-2 equiv.). The reaction mixture was stirred at room temperature overnight. After the reaction was complete as assessed by TLC analysis, the solvent was removed in vacuo and the crude product which was purified using CombiFlash chromatography eluting with MeOH/DCM to provide 18a-ac as a oily liquid or powder (109-330 mg, yield 30-98%). Final products were recrystallized from ethyl acetate and hexanes to afford high purity compounds.

### 4-(2-(Pyridin-4-yl)ethyl)-N-(4-((4(trifluoromethyl)benzyl)oxy)phenyl)piperazine-1-carboxamide (18c)

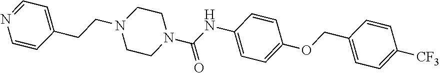 [SMILES: O=C(Nc1ccc(OCc2ccc(C(F)(F)F)cc2)cc1)N1CCN(CCc2ccncc2)CC1]

White solid, 240 mg, yield 80% from 180 mg of 17a (0.61 mmol); mp 137-138° C.; ¹H NMR (400 MHz, DMSO-d₆) 2.44 (brt, J=4.7 Hz, 4H), 2.56-2.61 (m, 2H), 2.75-2.81 (m, 2H), 3.40 (brt, J=4.6 Hz, 4H), 5.17 (s, 2H), 6.90 (d, J=9.1 Hz, 2H), 7.28 (d, J=5.9 Hz, 2H), 7.34 (d, J=9.0 Hz, 2H), 7.65 (d, J=8.1 Hz, 2H), 7.75 (d, J=8.2 Hz, 2H), 8.34 (s, 1H), 8.45 (d, J=5.9 Hz, 2H); ¹³C NMR (100 MHz, CDCl₃) S 32.9, 44.1, 52.9, 58.9, 69.6, 115.3, 122.4, 124.20 (q, J_{C-F}=272.3 Hz, overlapped with q at 125.6 ppm), 124.25, 125.6 (q, J_{C-F}=3.6 Hz), 127.5, 130.2 (q, J_{C-F}=32.3 Hz), 132.7, 141.3, 149.2, 149.9, 154.7, 155.5; HRMS m/z [M+H]⁺ calcd for C₂₆H₂₈F₃N₄O₂, 485.2159; found, 485.2157; Anal. Calcd for C₂₆H₂₇F₃N₄O₂: C, 64.45; H, 5.62; N, 11.56; Found: C, 64.47; H, 5.63; N, 11.55.

### N-(4-((3,5-bis(trifluoromethyl)benzyl)oxy)phenyl)-4-(pyridin-4-ylmethyl)piperazine-1-carboxamide (18d)

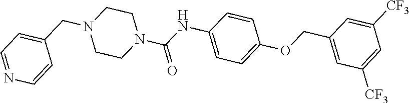 [SMILES: O=C(Nc1ccc(OCc2cc(C(F)(F)F)cc(C(F)(F)F)c2)cc1)N1CCN(Cc2ccncc2)CC1]

White solid, 280 mg, yield 75% from 250 mg of 17b (0.69 mmol); mp 170-171° C.; ¹H NMR (400 MHz, CDCl₃) 2.48 (t, J=5.1 Hz, 4H), 3.51 (t, J=5.1 Hz, 4H), 3.54 (s, 2H), 5.12 (s, 2H), 6.43 (s, 1H), 6.91 (d, J=9.0 Hz, 2H), 7.26-7.30 (m, 4H), 7.83 (s, 1H), 7.89 (s, 2H), 8.56 (d, J=6.0 Hz, 2H); ¹³C NMR (100 MHz, CDCl₃) δ 44.2, 52.9, 61.7, 68.9, 115.3, 121.8-122.0 (m, overlapped with q peak centered at 123.4), 122.4, 123.4 (q, J_{C-F}=272.6 Hz), 123.9, 127.2-127.4 (m), 132.0 (q, J_{C-F}=33.4 Hz), 133.1, 139.9, 147.2, 150.0, 154.3, 155.4; HRMS m/z [M+H]⁺ calcd for C₂₆H₂₅F₆N₄O₂, 539.1876; found, 539.1875; Elemental Analysis Calcd for C₂₆H₂₄F₆N₄O₂: C, 57.99; H, 4.49; N, 10.40; Found: C, 57.75; H, 4.66; N, 10.21.

### N-(4-((3,5-bis(trifluoromethyl)benzyl)oxy)phenyl)-4-(2-(pyridin-4-yl)ethyl)piperazine-1-carboxamide (18e)

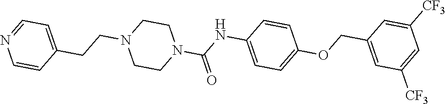 [SMILES: O=C(Nc1ccc(OCc2cc(C(F)(F)F)cc(C(F)(F)F)c2)cc1)N1CCN(CCc2ccncc2)CC1]

White solid, 330 mg, yield 86% from 250 mg of 17b (0.69 mmol); mp 91-92° C.; ¹H NMR (400 MHz, CDCl₃) 2.52 (t, J=5.1 Hz, 4H), 2.61-2.68 (m, 2H), 2.77-2.83 (m, 2H), 3.47-3.53 (m, 4H), 5.11 (s, 2H), 6.52 (brs, 1H), 6.90 (d, J=9.0 Hz, 2H), 7.14 (d, J=6.0 Hz, 2H), 7.28 (d, J=9.0, 2H, partially overlapped with residual solvent peak), 7.83 (s, 1H), 7.88 (s, 2H), 8.49 (d, J=6.0 Hz, 2H); ¹³C NMR (100 MHz, CDCl₃) δ 32.9, 44.2, 52.9, 58.9, 68.9, 115.3, 121.8-122.1 (m, overlapped with q peak centered at 123.4), 122.4, 123.4 (q, J_{C-F}=272.9 Hz), 124.3, 127.3 (b), 132.0 (q, J_{C-F}=33.5 Hz), 133.1, 139.9, 149.3, 149.8, 154.3, 155.5; HRMS m/z [M+H]⁺ calcd for C₂₇H₂₇F₆N₄O₂, 553.2033; found, 553.2030; Elemental Analysis Calcd for C₂₇H₂₆F₆N₄O₂: C, 58.69; H, 4.74; N, 10.14; Found: C, 58.78; H, 4.71; N, 10.18.

### N-(4-((3,5-dimethoxybenzyl)oxy)phenyl)-4-(pyridin-4-ylmethyl)piperazine-1-carboxamide (18f)

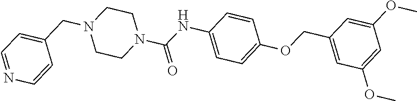 [SMILES: COc1cc(COc2ccc(NC(=O)N3CCN(Cc4ccncc4)CC3)cc2)cc(OC)c1]

Off-white powder, 170 mg, yield 52% starting from 200 mg of 17c (0.70 mmol); mp 123-124° C.; ¹H NMR (400 MHz, CDCl₃) δ 2.45 (t, J=5.0 Hz, 4H), 3.48 (t, J=5.0 Hz, 4H), 3.52 (s, 2H), 3.78 (s, 6H), 4.96 (s, 2H), 6.39 (t, J=2.3 Hz, 1H), 6.43 (br s, 1H), 6.56 (d, J=2.3 Hz, 2H), 6.88 (d, J=9.0 Hz, 2H), 7.22 (d, J=9.0 Hz, 2H), 7.27 (d, J=5.9 Hz, 2H), 8.54 (d, J=5.7 Hz, 2H). ¹³C NMR (100 MHz, CDCl₃) δ 44.2, 52.9, 55.5, 61.7, 70.4, 100.0, 105.3, 115.3, 122.4, 123.9, 132.4, 139.6, 147.3, 150.0, 155.1, 155.6, 161.1. HRMS m/z [M+H]⁺ calcd for C₂₆H₃₁N₄O₄, 463.23398; found, 463.23316; Elemental Analysis Calcd for C₂₆H₃₀N₄O₄: C, 67.51; H, 6.54; N, 12.11. Found: C, 67.36; H, 6.67; N, 11.88.

### N-(4-((3,5-dimethoxybenzyl)oxy)phenyl)-4-(2-(pyridin-4-yl)ethyl)piperazine-1-carboxamide (18g)

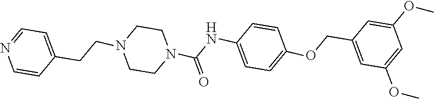 [SMILES: COc1cc(COc2ccc(NC(=O)N3CCN(CCc4ccncc4)CC3)cc2)cc(OC)c1]

White solid, 150 mg, yield 50% starting from 180 mg of 17c (0.63 mmol); mp 131-132° C.; ¹H NMR (400 MHz, CDCl₃) δ 2.50 (t, J=5.0 Hz, 4H), 2.60-2.66 (m, 2H), 2.76-2.82 (m, 2H), 3.48 (t, J=5.0 Hz, 4H), 3.77 (s, 6H), 4.95 (s, 2H), 6.39 (t, J=2.2 Hz, 1H), 6.48 (brs, 1H), 6.56 (d, J=2.2 Hz, 2H), 6.88 (d, J=8.9 Hz, 2H), 7.13 (d, J=5.8 Hz, 2H), 7.22 (m, J=8.9 Hz, 2H), 8.49 (d, J=5.9 Hz, 2H); ¹³C NMR (100 MHz, CDCl₃) δ 32.9, 44.1, 52.9, 55.5, 58.9, 70.4, 100.0, 105.3, 115.3, 122.4, 124.2, 132.4, 139.6, 149.3, 149.8, 155.1, 155.6, 161.1; HRMS m/z [M+H]+ calcd for C₂₇H₃₃N₄O₄, 477.2496; found, 477.2494; Elemental Analysis Calcd for C₂₇H₃₂N₄O₄: C, 68.05; H, 6.77; N, 11.76; Found: C, 67.85; H, 6.81; N, 11.75.

### N-(4-((3,5-di-tert-butylbenzyl)oxy)phenyl)-4-(pyridin-4-ylmethyl)piperazine-1-carboxamide (18h)

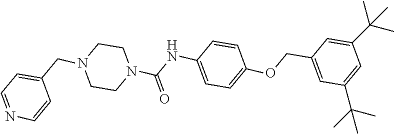 [SMILES: CC(C)(C)c1cc(COc2ccc(NC(=O)N3CCN(Cc4ccncc4)CC3)cc2)cc(C(C)(C)C)c1]

White solid, 260 mg, yield 63% from 270 mg of 17d (0.80 mmol); mp 145-146° C.; ¹H NMR (400 MHz, DMSO-d₆) 1.29 (s, 18H), 2.39 (t, J=4.9 Hz, 4H), 3.42-3.46 (m, 4H), 3.55 (s, 2H), 4.99 (s, 2H), 6.90 (d, J=9.1 Hz, 2H), 7.26 (d, J=1.8 Hz, 2H), 7.31-7.36 (m, 5H), 8.34 (s, 1H), 8.52 (d, J=5.9 Hz, 2H); ¹³C NMR (100 MHz, DMSO-d₆) δ 31.2, 34.5, 43.7, 52.5, 60.5, 70.2, 114.5, 121.3, 121.4, 122.0, 123.8, 133.6, 136.3, 147.2, 149.6, 150.3, 153.7, 155.2; HRMS m/z [M+H]⁺ calcd for C₃₂H₄₃N₄O₂, 515.3381; found, 515.3377; Elemental Analysis Calcd for C₃₂H₄₂N₄O₂: C, 74.67; H, 8.23; N, 10.89; Found: C, 74.79; H, 8.30; N, 10.88.

### N-(4-((3,5-di-tert-butylbenzyl)oxy)phenyl)-4-(2-(pyridin-4-yl)ethyl)piperazine-1-carboxamide (18i)

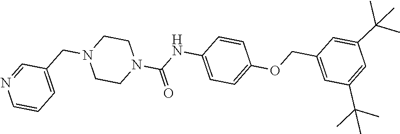 [SMILES: CC(C)(C)c1cc(COc2ccc(NC(=O)N3CCN(Cc4cccnc4)CC3)cc2)cc(C(C)(C)C)c1]

White solid, 305 mg, yield 78% from 250 mg of 17d (0.74 mmol); mp 165-166° C.; ¹H NMR (400 MHz, DMSO-d₆) 1.29 (s, 18H), 2.44 (t, J=4.9 Hz, 4H), 2.56-2.61 (m, 2H), 2.76-2.81 (m, 2H), 3.38-3.43 (m, 4H), 4.99 (s, 2H), 6.90 (d, J=9.1 Hz, 2H), 7.25-7.29 (m, 4H), 7.31-7.36 (m, 3H), 8.33 (s, 1H), 8.45 (d, J=6.0 Hz, 2H); ¹³C NMR (100 MHz, CDCl₃) δ 31.6, 32.9, 35.0, 44.2, 52.9, 58.9, 71.4, 115.3, 122.23, 122.29, 122.37, 124.3, 132.2, 136.1, 149.3, 149.9, 151.1, 155.5, 155.6; HRMS m/z [M+H]⁺ calcd for C₃₃H₄₅N₄O₂, 529.3537; found, 529.3534; Elemental Analysis Calcd for C₃₃H₄₄N₄O₂: C, 74.96; H, 8.39; N, 10.60; Found: C, 74.99; H, 8.56; N, 10.62.

### 4-(Pyridin-4-ylmethyl)-N-(4-((3-(trifluoromethoxy)benzyl)oxy)phenyl)piperazine-1-carboxamide (18j)

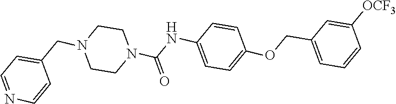 [SMILES: O=C(Nc1ccc(OCc2cccc(OC(F)(F)F)c2)cc1)N1CCN(Cc2ccncc2)CC1]

White solid, 230 mg, yield 52% from 280 mg of 17e (0.91 mmol); mp 131-132° C.; ¹H NMR (400 MHz, DMSO-d₆) δ 2.39 (t, J=4.9 Hz, 4H), 3.41-3.46 (m, 4H), 3.55 (s, 2H), 5.11 (s, 2H), 6.90 (d, J=9.1 Hz, 2H), 7.29-7.36 (m, 5H), 7.42 (br s, 1H), 7.45-7.49 (m, 1H), 7.53 (br t, J=7.8 Hz, 1H), 8.35 (s, 1H), 8.52 (d, J=5.9 Hz, 2H); ¹³C NMR (100 MHz, CDCl₃) δ 44.2, 52.9, 61.7, 69.5, 115.3, 119.9, 120.4, 120.6 (q, J_{C-F}=257.2 Hz), 122.4, 123.9, 125.6, 130.1, 132.6, 139.7, 147.3, 149.5-149.6 (m), 150.1, 154.8, 155.5; HRMS m/z [M+H]⁺ calcd for C₂₅H₂₆F₃N₄O₃, 487.1952; found, 487.1946; Elemental Analysis Calcd for C₂₅H₂₅F₃N₄O₃: C, 61.72; H, 5.18; N, 11.52, Found: C, 61.85; H, 5.19; N, 11.43.

### 4-(2-(pyridin-4-yl)ethyl)-N-(4-((3-(trifluoromethoxy)benzyl)oxy)phenyl)piperazine-1-carboxamide (18k)

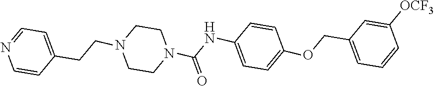 [SMILES: O=C(Nc1ccc(OCc2cccc(OC(F)(F)F)c2)cc1)N1CCN(CCc2ccncc2)CC1]

White solid, 250 mg, yield 70% from 220 mg of 17e (0.71 mmol); mp 112-113° C.; ¹H NMR (400 MHz, DMSO-d₆) δ 2.41-2.46 (m, 4H), 2.56-2.61 (m, 2H), 2.75-2.81 (m, 2H), 3.38-3.42 (m, 4H), 5.11 (s, 2H), 6.90 (d, J=9.1 Hz, 2H), 7.26-7.36 (m, 5H), 7.42 (brs, 1H), 7.45-7.49 (m, 1H), 7.53 (t, J=7.8 Hz, 1H), 8.34 (s, 1H), 8.45 (d, J=6.0 Hz, 2H); ¹³C NMR (100 MHz, CDCl₃) δ 32.9, 44.2, 52.9, 58.9, 69.6, 115.4, 119.9, 120.3, 120.6 (q, J_{C-F}=257.6 Hz), 122.4, 124.2, 125.6, 130.1, 132.6, 139.7, 149.2, 149.6 (br), 149.9, 154.8, 155.5; HRMS m/z [M+H]⁺ calcd for C₂₆H₂₈F₃N₄O₃, 501.2108; found, 501.2103; Elemental Analysis Calcd for C₂₆H₂₇F₃N₄O₃: C, 62.39; H, 5.44; N, 11.19; Found: C, 62.41; H, 5.44; N, 11.22.

### N-(4-((3,5-bis(trifluoromethyl)benzyl)oxy)phenyl)-4-(2-(pyridin-3-yl)ethyl)piperazine-1-carboxamide (18l)

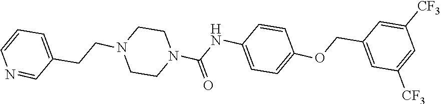 [SMILES: O=C(Nc1ccc(OCc2cc(C(F)(F)F)cc(C(F)(F)F)c2)cc1)N1CCN(CCc2cccnc2)CC1]

Light orange/white crystals, 221 mg, yield 73% starting from 200 mg of 17b (0.55 mmol); mp 146-147° C.; ¹H NMR (400 MHz, CDCl₃) δ 2.54 (t, J=5.0 Hz, 4H), 2.60-2.66 (m, 2H), 2.77-2.84 (m, 2H), 3.50 (t, J=5.0 Hz, 4H), 5.11 (s, 2H), 6.45 (br s, 1H), 6.91 (d, J=9.0 Hz, 2H), 7.22 (dd, J=7.7, 4.8 Hz, 1H), 7.29 (d, J=9.0 Hz, 2H), 7.53 (br d, J=7.8 Hz, H), 7.83 (s, 1H), 7.88 (s, 2H), 8.41-8.53 (m, 2H). ¹³C NMR (100 MHz, CDCl₃) δ 30.7, 44.2, 52.9, 59.6, 69.0, 115.3, 121.9 (septet, J=3.8 Hz, overlapped with q signal centered at 123.4), 122.4, 123.4 (q, J=272.7 Hz), 123.5, 127.3-127.4 (m), 132.0 (q, J=33.4 Hz), 133.2, 135.6, 136.3, 139.9, 147.8, 150.2, 154.4, 155.5. HRMS m/z [M+H]⁺ calcd for C₂₇H₂₇F₆N₄O₂, 533.20327; found, 553.20304; Elemental Analysis Calcd for C₂₇H₂₆F₆N₄O₂: C, 58.69; H, 4.74; N, 10.14. Found: C, 58.75; H, 4.93; N, 10.13.

### 4-(2-(1H-pyrrol-1-yl)ethyl)-N-(4-((3,5-bis(trifluoromethyl)benzyl)oxy)phenyl)piperazine-1-carboxamide (18m)

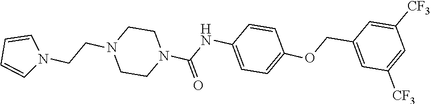 [SMILES: O=C(Nc1ccc(OCc2cc(C(F)(F)F)cc(C(F)(F)F)c2)cc1)N1CCN(CCn2cccc2)CC1]

White solid, 320 mg, yield 82% from 260 mg of 17b (0.72 mmol); mp 104-105° C.; ¹H NMR (400 MHz, CDCl₃) 2.47 (t, J=5.1 Hz, 4H), 2.74 (t, J=6.7 Hz, 2H), 3.45-3.50 (m, 4H), 4.02 (t, J=6.7 Hz, 2H), 5.12 (s, 2H), 6.15 (t, J=2.1 Hz, 2H), 6.32 (s, 1H), 6.70 (t, J=2.1 Hz, 2H), 6.91 (d, J=9.0 Hz, 2H), 7.28 (d, J=9.0 Hz, 2H), 7.84 (s, 1H), 7.89 (s, 2H); ¹³C NMR (100 MHz, CDCl₃) δ 44.2, 47.6, 53.1, 59.2, 69.0, 108.4, 115.3, 120.9, 121.8-122.0 (m, overlapped with q signal centered at 123.4), 122.4, 123.4 (q, J_{C-F}=272.7 Hz), 127.2-127.4 (m), 132.0 (q, J_{C-F}=33.4 Hz), 133.1, 139.9, 154.4, 155.4; HRMS m/z [M+H]⁺ calcd for C₂₆H₂₇F₆N₄O₂, 541.2033; found, 541.2026; Elemental Analysis Calcd for C₂₆H₂₆F₆N₄O₂: C, 57.78; H, 4.85; N, 10.37; Found: C, 57.74; H, 4.84; N, 10.33.

### N-(4-((3,5-bis(trifluoromethyl)benzyl)oxy)phenyl)-4-(2-(2,5-dimethyl-1H-pyrrol-1-yl)ethyl)piperazine-1-carboxamide (18n)

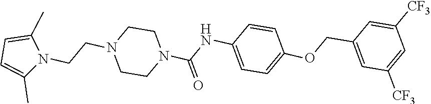 [SMILES: Cc1ccc(C)n1CCN1CCN(C(=O)Nc2ccc(OCc3cc(C(F)(F)F)cc(C(F)(F)F)c3)cc2)CC1]

Off-white powder, 109 mg, yield 46% starting from 150 mg of 17b (0.42 mmol); mp 93-98° C.; ¹H NMR (400 MHz, CDCl₃) δ 2.25 (s, 6H), 2.51 (t, J=5.0 Hz, 4H), 2.54-2.59 (m, 2H), 3.49 (t, J=5.0 Hz, 4H), 3.87-3.93 (m, 2H), 5.13 (s, 2H), 5.77 (s, 2H), 6.29 (s, 1H), 6.92 (d, J=9.0 Hz, 2H), 7.28 (d, J=9.0 Hz, 2H), 7.84 (br s, 1H), 7.90 (br s, 2H). ¹³C NMR (100 MHz, CDCl₃) 12.7, 41.7, 44.2, 53.5, 58.4, 69.0, 105.6, 115.4, 121.9-122.1 (m), 122.4, 123.4 (q, J=272.6 Hz), 127.3-127.4 (m), 127.6, 132.0 (q, J=33.4 Hz), 133.1, 139.9, 154.4, 155.4. HRMS: m/z [M+H]⁺ calcd for C₂₈H₃₁F₆N₄O₂, 569.23457; found, 569.23436; Elemental Analysis Calcd for C₂₈H₃₀F₆N₄O₂: C, 59.15; H, 5.32; N, 9.85. Found: C, 59.04; H, 5.47; N, 9.84.

### 4-(2-(1H-pyrazol-1-yl)ethyl)-N-(4-((3,5-bis(trifluoromethyl)benzyl)oxy)phenyl)piperazine-1-carboxamide (18o)

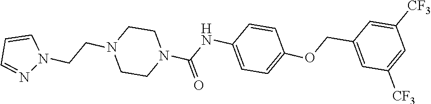 [SMILES: O=C(Nc1ccc(OCc2cc(C(F)(F)F)cc(C(F)(F)F)c2)cc1)N1CCN(CCn2cccn2)CC1]

White powder, 115 mg, yield 51% starting from 150 mg of 17b (0.42 mmol); mp 144-145° C.; ¹H NMR (400 MHz, DMSO-d₆) δ 2.41 (t, J=4.9 Hz, 4H), 2.73 (t, J=6.7 Hz, 2H), 3.36-3.42 (m, 4H), 4.24 (t, J=6.6 Hz, 2H), 5.26 (s, 2H), 6.21 (t, J=2.0 Hz, 1H), 6.94 (d, J=9.1 Hz, 2H), 7.36 (d, J=9.1 Hz, 2H), 7.41-7.43 (m, 1H), 7.72-7.75 (m, 1H), 8.07 (br s, 1H), 8.14 (br s, 2H), 8.35 (s, 1H). ¹³C NMR (100 MHz, CDCl₃) δ 44.2, 50.0, 53.0, 57.9, 69.0, 105.6, 115.3, 121.9-122.1 (m), 122.4, 123.4 (q, J=272.7 Hz), 127.2-127.4 (m), 129.6, 132.0 (q, J=33.4 Hz), 133.1, 139.5, 139.9, 154.4, 155.4. HRMS: m/z [M+H]⁺ calcd for C₂₅H₂₆F₆N₅O₂, 542.19852; found, 542.19802; Elemental Analysis Calcd for C₂₅H₂₅F₆N₅O₂: C, 55.45; H, 4.65; N, 12.93. Found: C, 55.37; H, 4.73; N, 13.01.

### 4-(2-(1H-imidazol-1-yl)ethyl)-N-(4-((3,5-bis(trifluoromethyl)benzyl)oxy)phenyl)piperazine-1-carboxamide (18p)

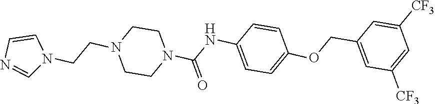 [SMILES: O=C(Nc1ccc(OCc2cc(C(F)(F)F)cc(C(F)(F)F)c2)cc1)N1CCN(CCn2ccnc2)CC1]

White solid, 120 mg, yield 58% from 138 mg of 17b (0.38 mmol); mp 174-175° C.; ¹H NMR (400 MHz, CDCl₃) 2.48 (t, J=4.9 Hz, 4H), 2.72 (t, J=6.2 Hz, 2H), 3.45-3.51 (m, 4H, overlapped with diethyl ether solvent peak), 4.05 (t, 0.1=6.1 Hz, 2H), 5.12 (s, 2H), 6.55 (s, 1H), 6.91 (d, J=8.9 Hz, 2H), 6.97 (s, 1H), 7.05 (s, 1H), 7.28 (d, J=8.9 Hz, 2H), 7.54 (s, 1H), 7.83 (s, 1H), 7.89 (s, 2H); ¹³C NMR (100 MHz, CDCl₃) δ 44.2, 44.9, 53.1, 58.6, 68.9, 115.3, 119.3 (overlapped with q peak centered at 123.4), 121.8-122.0 (m, overlapped with q peak centered at 123.4), 122.4, 123.4 (q, J_{C-F}=272.6 Hz), 127.3-127.4 (m), 129.5, 132.0 (q, J_{C-F}=33.4 Hz), 133.1, 137.5, 139.9, 154.3, 155.4; HRMS m, [M+H]⁺ calcd for C₂₅H₂₆F₆N₅O₂, 542.1985; found, 542.1982; Elemental Analysis Calcd for C₂₅H₂₅F₆N₅O₂: C, 55.45; H, 4.65; N, 12.93; Found: C, 55.51; H, 4.83; N, 12.76.

### 4-(2-(1H-1,2,4-triazol-1-yl)ethyl)-N-(4-((3,5-bis(trifluoromethyl)benzyl)oxy)phenyl)piperazine-1-carboxamide (18q)

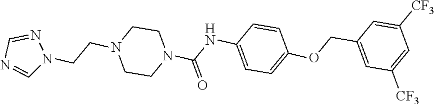 [SMILES: O=C(Nc1ccc(OCc2cc(C(F)(F)F)cc(C(F)(F)F)c2)cc1)N1CCN(CCn2cncn2)CC1]

White solid, 120 mg, yield 30% from 270 mg of 17b (0.75 mmol); mp 179-180° C.; ¹H NMR (400 MHz, DMSO-d₅) 2.42 (t, 1=4.9 Hz, 4H), 2.73 (t, J=6.3 Hz, 2H), 3.35-3.40 (m, 4H), 4.32 (t, J=6.3 Hz, 2H), 5.26 (s, 2H), 6.94 (d, J=9.1 Hz, 2H), 7.35 (d, J=9.1 Hz, 2H), 7.94 (s, 1H), 8.08 (br s, 1H), 8.14 (br s, 2H), 8.35 (s, 1H), 8.51 (s, 1H); ¹³C NMR (100 MHz, CDCl₃) δ 44.2, 47.5, 52.9, 57.0, 69.0, 115.4, 121.9-122.1 (m), 122.4, 123.4 (q, J_{C-F}=272.6 Hz), 127.3-127.5 (m), 132.0 (q, J_{C-F}=33.4 Hz), 133.0, 139.9, 143.6, 151.9, 154.4, 155.3; HRMS m/z [M+H]⁺ calcd for C₂₄H₂₅F₆N₆O₂, 543.1938; found, 543.1934; Elemental Analysis Calcd for C₂₄H₂₄F₆N₆O₂: C, 53.14; H, 4.46; N, 15.49; Found: C, 53.03; H, 4.48; N, 15.38.

### N-(4-((3,5-bis(trifluoromethyl)benzyl)oxy)phenyl)-4-phenethylpiperazine-1-carboxamide (18r)

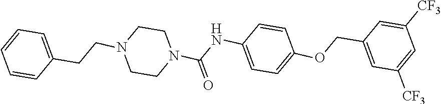 [SMILES: O=C(Nc1ccc(OCc2cc(C(F)(F)F)cc(C(F)(F)F)c2)cc1)N1CCN(CCc2ccccc2)CC1]

Off-white powder, 167 mg, yield 69% starting from 160 mg of 17b (0.44 mmol); mp 130-131° C.; ¹H NMR (400 MHz, CDCl₃) δ 2.54-2.59 (m, 4H), 2.62-2.68 (m, 2H), 2.80-2.86 (m, 2H), 3.49-3.56 (m, 4H), 5.13 (s, 2H), 6.29 (s, 1H), 6.92 (d, J=9.0 Hz, 2H), 7.18-7.24 (m, 3H), 7.27-7.32 (m, 4H), 7.84 (s, 1H), 7.90 (s, 2H). ¹³C NMR (100 MHz, CDCl₃) S 33.7, 44.2, 53.0, 60.4, 69.0, 115.4, 121.8-122.1 (m), 122.3, 123.4 (q, J_{C-F}=272.8 Hz), 126.3, 127.3-127.4 (m), 128.6, 128.8, 132.0 (q, J_{C-F}=33.4 Hz), 133.1, 140.0, 140.2, 154.4, 155.4. HRMS: m/z [M+H]⁺ calcd for C₂₈H₂₈FN₃O₂, 552.20802; found, 552.20783; Elemental Analysis Calcd for C₂₈H₂₇F₆N₃O₂: C, 60.98; H, 4.93; N, 7.62. Found: C, 61.10; H, 5.05; N, 7.65.

### N-(4-((3,5-bis(trifluoromethyl)benzyl)oxy)phenyl)-4-(2-methylphenethyl)piperazine-1-carboxamide (18s)

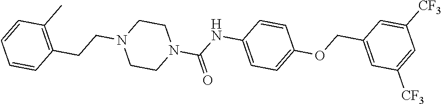 [SMILES: Cc1ccccc1CCN1CCN(C(=O)Nc2ccc(OCc3cc(C(F)(F)F)cc(C(F)(F)F)c3)cc2)CC1]

White powder, 223 mg, yield 94% starting from 150 mg of 17b (0.42 mmol); mp 76-88° C.; ¹H NMR (400 MHz, CDCl₃) δ 2.34 (s, 3H), 2.55-2.62 (m, 6H), 2.79-2.87 (m, 2H), 3.50-3.58 (m, 4H), 5.12 (s, 2H), 6.32 (s, 1H), 6.90-6.96 (m, 2H), 7.09-7.18 (m, 4H), 7.27-7.33 (m, 2H), 7.84 (br s, 1H), 7.90 (br s, 2H). ¹³C NMR (100 MHz, CDCl₃) δ 19.5, 30.9, 44.2, 53.0, 59.2, 69.0, 115.3, 122.0 (m), 122.4, 123.4 (q, J=272.7 Hz), 126.2, 126.5, 127.3 (m), 129.4, 130.4, 132.0 (q, J=33.4 Hz), 133.1, 136.1, 138.2, 140.0, 154.4, 155.4. HRMS: m/z [M+H]⁺ calcd for C₂₉H₃₀F₆N₃O₂, 566.22367; found, 566.22294; Elemental Analysis Calcd for C₂₉H₂₉F₆N₃O₂: C, 61.59; H, 5.17; N, 7.43. Found: C, 61.68; H, 5.39; N, 7.48.

### N-(4-((3,5-bis(trifluoromethyl)benzyl)oxy)phenyl)-4-(3-methylphenethyl)piperazine-1-carboxamide (18t)

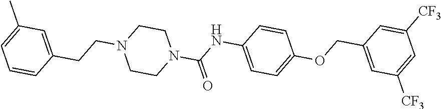 [SMILES: Cc1cccc(CCN2CCN(C(=O)Nc3ccc(OCc4cc(C(F)(F)F)cc(C(F)(F)F)c4)cc3)CC2)c1]

White powder, 110 mg, yield 46% starting from 150 mg of 17b (0.42 mmol); mp 112-113° C.; ¹H NMR (400 MHz, CDCl₃) δ 2.33 (s, 3H), 2.53-2.59 (m, 4H), 2.60-2.67 (m, 2H), 2.75-2.83 (m, 2H), 3.49-3.57 (m, 4H), 5.12 (s, 2H), 6.31 (s, 1H), 6.89-6.95 (m, 2H), 6.98-7.05 (m, 3H), 7.15-7.22 (m, 1H), 7.27-7.33 (m, 2H), 7.84 (br s, 1H), 7.90 (br s, 2H). ¹³C NMR (100 MHz, CDCl₃) δ 21.5, 33.6, 44.2, 53.0, 60.5, 69.0, 115.3, 122.0 (m), 122.3, 123.4 (q, J=273.0 Hz), 125.8, 127.1, 127.3 (m), 128.5, 129.6, 132.0 (q, J=33.4 Hz), 133.1, 138.2, 140.0, 140.1, 154.4, 155.4. HRMS: m/z [M+H]⁺ calcd for C₂₉H₃₀F₆N₃O₂, 566.22367; found, 566.22324; Elemental Analysis Calcd for C₂₉H₂₉F₆N₃O₂: C, 61.59; H, 5.17; N, 7.43. Found: C, 61.48; H, 5.43; N, 7.48.

### N-(4-((3,5-bis(trifluoromethyl)benzyl)oxy)phenyl)-4-(4-methylphenethyl)piperazine-1-carboxamide (18u)

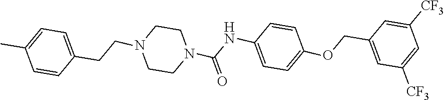 [SMILES: Cc1ccc(CCN2CCN(C(=O)Nc3ccc(OCc4cc(C(F)(F)F)cc(C(F)(F)F)c4)cc3)CC2)cc1]

White powder, 185 mg, yield 59% starting from 200 mg of 17b (0.55 mmol); mp 130-131° C.; ¹H NMR (400 MHz, CDCl₃) δ 2.32 (s, 3H), 2.52-2.59 (m, 4H), 2.59-2.66 (m, 2H), 2.75-2.82 (m, 2H), 3.48-3.56 (m, 4H), 5.13 (s, 2H), 6.29 (s, 1H), 6.90-6.95 (m, 2H), 7.10 (s, 4H), 7.27-7.32 (m, 2H), 7.84 (br s, 1H), 7.90 (br s, 2H). ¹³C NMR (100 MHz, CDCl₃) δ 21.1, 33.2, 44.2, 53.0, 60.6, 69.0, 115.4, 121.9-122.0 (m), 122.3, 123.4 (q, J=272.8 Hz), 127.3-127.4 (m), 128.7, 129.3, 132.0 (q, J=33.5 Hz), 133.1, 135.8, 137.0, 140.0, 154.4, 155.4. HRMS: m/z [M+H]⁺ calcd for C₂₉H₃₀F₆N₃O₂, 566.22367; found, 566.22338; Elemental Analysis Calcd for C₂₉H₂₉F₆N₃O₂: C, 61.59; H, 5.17; N, 7.43. Found: C, 61.52; H, 5.30; N, 7.57.

### N-(4-((3,5-bis(trifluoromethyl)benzyl)oxy)phenyl)-4-(2-methoxyphenethyl)piperazine-1-carboxamide (18v)

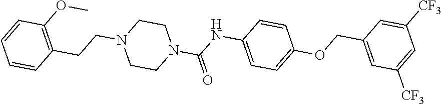 [SMILES: COc1ccccc1CCN1CCN(C(=O)Nc2ccc(OCc3cc(C(F)(F)F)cc(C(F)(F)F)c3)cc2)CC1]

White powder, 140 mg, yield 58% starting from 150 mg of 17b (0.42 mmol); mp 123-124° C.; H NMR (400 MHz, DMSO-d₆) δ 2.39-2.49 (m, 6H, overlapped with solvent peak), 2.69-2.77 (m, 2H), 3.38-3.46 (m, 4H), 3.78 (s, 3H), 5.26 (s, 2H), 6.86 (td, 0.1=7.4, 1.0 Hz, 1H), 6.91-6.97 (m, 3H), 7.13-7.20 (m, 2H), 7.34-7.40 (m, 2H), 8.07 (br s, 1H), 8.14 (br s, 2H), 8.36 (s, 1H). ¹³C NMR (100 MHz, CDCl₃) δ 28.0, 44.2, 52.9, 55.4, 58.7, 69.0, 110.5, 115.3, 120.6, 121.9 (m), 122.3, 123.4 (q, J=272.8 Hz), 127.3 (m), 127.6, 128.5, 130.3, 132.0 (q, J=33.4 Hz), 133.2, 140.0, 154.3, 155.4, 157.6. HRMS: m/z [M+H]⁺ calcd for C₂₉H₃₀F₆N₃O₃, 582.21859; found, 582.21789; Elemental Analysis Calcd for C₂₉H₂₉F₆N₃O₃: C, 59.89; H, 5.03; N, 7.23. Found: C, 59.78; H, 5.09; N, 7.25.

### N-(4-((3,5-bis(trifluoromethyl)benzyl)oxy)phenyl)-4-(3-methoxyphenethyl)piperazine-1-carboxamide (18w)

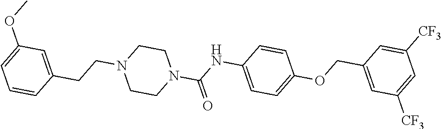 [SMILES: COc1cccc(CCN2CCN(C(=O)Nc3ccc(OCc4cc(C(F)(F)F)cc(C(F)(F)F)c4)cc3)CC2)c1]

White powder, 158 mg, yield 65% starting from 150 mg of 17b (0.42 mmol); mp 101-103° C.; ¹H NMR (400 MHz, CDCl₃) δ 2.53-2.59 (m, 4H), 2.61-2.68 (m, 2H), 2.76-2.84 (m, 2H), 3.49-3.56 (m, 4H), 3.80 (s, 3H), 5.13 (s, 2H), 6.29 (s, 1H), 6.73-6.78 (m, 2H), 6.78-6.82 (m, 1H), 6.89-6.96 (m, 2H), 7.18-7.24 (m, 1H), 7.27-7.32 (m, 2H), 7.84 (br s, 1H), 7.89 (br s, 2H). ¹³C NMR (100 MHz, CDCl₃) δ 33.7, 44.2, 53.0, 55.3, 60.3, 69.0, 111.5, 114.7, 115.4, 121.2, 121.9-122.0 (m), 122.3, 123.4 (q, J=272.8 Hz), 127.3-127.5 (m), 129.6, 132.0 (q, J=33.4 Hz), 133.1, 140.0, 141.8, 154.4, 155.4, 159.8. HRMS: m/z [M+H]⁺ calcd for C₂₉H₃₀F₆N₃O₃, 582.21859; found, 582.21826; Elemental Analysis Calcd for C₂₉H₂₉F₆N₃O₃: C, 59.89; H, 5.03; N, 7.23. Found: C, 59.75; H, 5.06; N, 7.14.

### N-(4-((3,5-bis(trifluoromethyl)benzyl)oxy)phenyl)-4-(4-methoxyphenethyl)piperazine-1-carboxamide (18x)

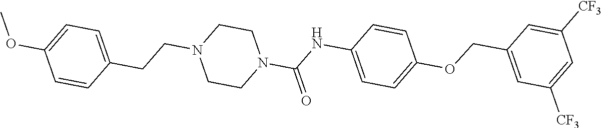 [SMILES: COc1ccc(CCN2CCN(C(=O)Nc3ccc(OCc4cc(C(F)(F)F)cc(C(F)(F)F)c4)cc3)CC2)cc1]

White powder, 143 mg, yield 59% starting from 150 mg of 17b (0.42 mmol); mp 122-123° C.; ¹H NMR (400 MHz, CDCl₃) δ 2.51-2.57 (m, 4H), 2.57-2.64 (m, 2H), 2.72-2.80 (m, 2H), 3.48-3.56 (m, 4H), 3.79 (s, 3H), 5.12 (s, 2H), 6.33 (s, 1H), 6.81-6.86 (m, 2H), 6.89-6.95 (m, 2H), 7.09-7.15 (m, 2H), 7.27-7.32 (m, 2H), 7.84 (br s, 1H), 7.89 (br s, 2H). ¹³C NMR (100 MHz, CDCl₃) δ 32.7, 44.2, 53.0, 55.4, 60.7, 69.0, 114.0, 115.3, 121.8-122.0 (m), 122.4, 123.4 (q, J=272.7 Hz), 127.2-127.5 (m), 129.7, 132.0 (q, J=33.4 Hz), 132.2, 133.1, 140.0, 154.4, 155.4, 158.2. HRMS: m/z [M+H]⁺ calcd for C₂₉H₃₀F₆N₃O₃, 582.21859; found, 582.21829; Elemental Analysis Calcd for C₂₉H₂₉F₆N₃O₃: C, 59.89; H, 5.03; N, 7.23. Found: C, 59.72; H, 5.07; N, 7.18.

### N-(4-((3,5-bis(trifluoromethyl)benzyl)oxy)phenyl)-4-(2-chlorophenethyl)piperazine-1-carboxamide (18y)

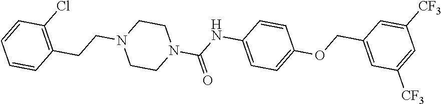 [SMILES: O=C(Nc1ccc(OCc2cc(C(F)(F)F)cc(C(F)(F)F)c2)cc1)N1CCN(CCc2ccccc2Cl)CC1]

White powder, 154 mg, yield 69% starting from 137 mg of 17b (0.38 mmol); mp 110-111° C.; ¹H NMR (400 MHz, DMSO-d₆) δ 2.43-2.48 (m, 4H, overlapped with DMSO peak), 2.51-2.57 (m, 2H, overlapped with DMSO peak), 2.85-2.92 (m, 2H), 3.39-3.45 (m, 4H), 5.26 (s, 2H), 6.91-6.97 (m, 2H), 7.21-7.30 (m, 2H), 7.34-7.43 (m, 4H), 8.07 (br s, 1H), 8.14 (br s, 2H), 8.36 (s, 1H). ¹³C NMR (100 MHz, CDCl₃) δ 31.2, 44.2, 52.9, 58.4, 69.0, 115.3, 121.9-122.0 (m), 122.4, 123.4 (q, J=272.7 Hz), 127.0, 127.3-127.4 (m), 127.9, 129.7, 130.9, 132.0 (q, J=33.4 Hz), 133.2, 134.1, 137.7, 140.0, 154.4, 155.4. HRMS: m/z: [M+H]+ calcd for C₂₈H₂₇ClF₆N₃O₂, 586.16905; found, 586.16916; Elemental Analysis Calcd for C₂₈H₂₆ClF₆N₃O₂: C, 57.39; H, 4.47; N, 7.17. Found: C, 57.35; H, 4.55; N, 7.21.

### N-(4-((3,5-bis(trifluoromethyl)benzyl)oxy)phenyl)-4-(3-chlorophenethyl)piperazine-1-carboxamide (18z)

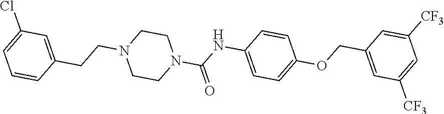 [SMILES: O=C(Nc1ccc(OCc2cc(C(F)(F)F)cc(C(F)(F)F)c2)cc1)N1CCN(CCc2cccc(Cl)c2)CC1]

Off-white powder, 120 mg, yield 74% starting from 100 mg of 17b (0.28 mmol); mp 133-134° C.; ¹H NMR (400 MHz, CDCl₃) δ 2.51-2.58 (m, 4H), 2.59-2.67 (m, 2H), 2.75-2.83 (m, 2H), 3.48-3.55 (m, 4H), 5.12 (s, 2H), 6.33 (s, 1H), 6.89-6.95 (m, 2H), 7.06-7.11 (m, 1H), 7.16-7.25 (m, 3H), 7.27-7.33 (m, 2H), 7.84 (br s, 1H), 7.89 (br s, 2H). ¹³C NMR (100 MHz, CDCl₃) δ 33.3, 44.2, 52.9, 59.9, 69.0, 115.3, 122.0 (m), 122.4, 123.4 (q, J=272.7 Hz), 126.5, 127.0, 127.3-127.4 (m), 128.9, 129.8, 132.0 (q, J=33.4 Hz), 133.1, 134.3, 140.0, 142.2, 154.4, 155.4. HRMS m/z [M+H]⁺ calcd for C₂₈H₂₇ClF₆N₃O₂, 586.16905; found, 586.16942; Elemental Analysis Calcd for C₂₈H₂₆ClF₆N₃O₂: C, 57.39; H, 4.47; N, 7.17. Found: C, 57.36; H, 4.59; N, 7.22.

### N-(4-((3,5-bis(trifluoromethyl)benzyl)oxy)phenyl)-4-(4-chlorophenethyl)piperazine-1-carboxamide (18aa)

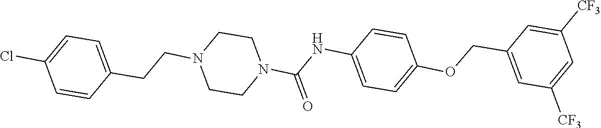 [SMILES: O=C(Nc1ccc(OCc2cc(C(F)(F)F)cc(C(F)(F)F)c2)cc1)N1CCN(CCc2ccc(Cl)cc2)CC1]

Off-white/yellow powder, 159 mg, yield 98% starting from 100 mg of 17b (0.28 mmol); mp 106-108° C.; ¹H NMR (400 MHz, DMSO-d₆) δ 2.39-2.47 (m, 4H), 2.50-2.57 (m, 2H, overlapped with DMSO peak), 2.71-2.79 (m, 2H), 3.37-3.45 (m, 4H), 5.26 (s, 2H), 6.89-7.00 (m, 2H), 7.24-7.29 (m, 2H), 7.30-7.41 (m, 4H), 8.07 (br s, 1H), 8.14 (br s, 2H), 8.36 (s, 1H). ¹³C NMR (100 MHz, DMSO-d₆) δ 31.8, 43.7, 52.5, 59.2, 67.8, 114.6, 121.4, 121.5, 123.3 (q, J=272.8 Hz), 128.1, 128.1 (m, overlapped), 130.3 (q, J=32.9 Hz), 130.4, 130.5, 134.2, 139.5, 141.0, 152.9, 155.2. HRMS m/z [M+H]⁺ calcd for C₂₈H₂₇ClF₆N₃O₂, 586.16905; found, 586.16881; Elemental Analysis Calcd for C₂₈H₂₆ClF₆N₃O₂: C, 57.39; H, 4.47; N, 7.17. Found: C, 57.11; H, 4.53; N, 7.18.

### N-(4-((3,5-bis(trifluoromethyl)benzyl)oxy)phenyl)-4-(3-phenylpropyl)piperazine-1-carboxamide (18ab)

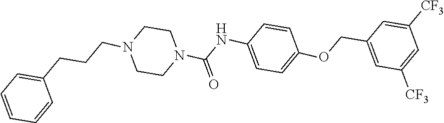 [SMILES: O=C(Nc1ccc(OCc2cc(C(F)(F)F)cc(C(F)(F)F)c2)cc1)N1CCN(CCCc2ccccc2)CC1]

White powder, 144 mg, yield 62% starting from 150 mg of 17b (0.41 mmol); mp 138-139° C.; ¹H NMR (400 MHz, DMSO-d₆) δ 1.75 (quint, J=7.48 Hz, 2H), 2.27-2.38 (m, 6H), 2.60 (t, J=7.84 Hz, 2H), 3.38-3.44 (m, 4H), 5.26 (s, 2H), 6.91-6.98 (m, 2H), 7.14-7.24 (m, 3H), 7.24-7.31 (m, 2H), 7.32-7.39 (m, 2H), 8.07 (br s, 1H), 8.14 (br s, 2H), 8.34 (s, 1H). ¹³C NMR (100 MHz, CDCl₃) δ 28.6, 33.7, 44.2, 53.0, 57.9, 69.0, 115.3, 121.8-122.0 (m), 122.4, 123.4 (q, J=272.7 Hz), 126.0, 127.3-127.4 (m), 128.48, 128.52, 132.0 (q, J=33.4 Hz), 133.2, 140.0, 142.1, 154.3, 155.4. HRMS: m/z [M+H]⁺ calcd for C₂₉H₃₀F₆N₃O₂, 566.22367; found, 566.22318; Elemental Analysis Calcd for C₂₉H₂₉F₆N₃O₂: C, 61.59; H, 5.17; N, 7.43. Found: C, 61.81; H, 5.18; N, 7.29.

### N-(4-((3,5-bis(trifluoromethyl)benzyl)oxy)phenyl)-4-(4-phenylbutyl)piperazine-1-carboxamide (18ac)

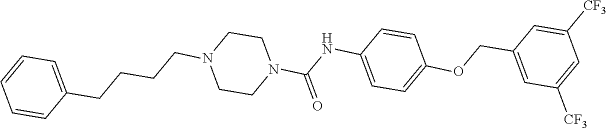 [SMILES: O=C(Nc1ccc(OCc2cc(C(F)(F)F)cc(C(F)(F)F)c2)cc1)N1CCN(CCCCc2ccccc2)CC1]

Off-white powder, 158 mg, yield 65% starting from 150 mg of 17b (0.42 mmol); mp 101-102° C.; ¹H NMR (400 MHz, DMSO-d₆) δ 1.40-1.50 (m, 2H), 1.53-1.64 (m, 2H), 2.25-2.37 (m, 6H), 2.59 (t, J=7.5 Hz, 2H), 3.35-3.44 (m, 4H), 5.26 (s, 2H), 6.91-6.97 (m, 2H), 7.13-7.22 (m, 3H), 7.24-7.30 (m, 2H), 7.32-7.40 (m, 2H), 8.07 (br s, 1H), 8.14 (br s, 2H), 8.34 (s, 1H). ¹³C NMR (100 MHz, CDCl₃) δ 26.5, 29.4, 35.9, 44.2, 53.0, 58.5, 69.0, 115.3, 121.8-122.0 (m), 122.3, 123.4 (q, J=272.7 Hz), 125.9, 127.3-127.5 (m), 128.4, 128.5, 132.0 (q, J=33.4 Hz), 133.2, 140.0, 142.5, 154.3, 155.4. HRMS: m/z [M+H]⁺ calcd for C₃₀H₃₂F₆N₃O₂, 580.23932; found, 580.23909; Elemental Analysis Calcd for C₃₀H₃₁F₆N₃O₂: C, 62.17; H, 5.39; N, 7.25. Found: C, 62.37; H, 5.44; N, 7.44.

### Biological Assays

Promastigote assay. The effect of the compounds on the proliferation of Leishmania donovani LV82 strain parasites (MHOM/ET/67:LV82) was carried out as outlined by Feng et al.²⁶

J774 macrophage assay. This assay was performed as reported by Zhu et al.²⁷

L. donovani-infected macrophage assay. The efficacy of selected target compounds against intracellular L. donovani LV82 parasites was conducted using murine peritoneal macrophages as the host cell in a high content imaging assay essentially as described by Joice et al.²⁴ and Abdelhameed et al.²⁸, with the following modifications. The density of peritoneal macrophages was 5×10⁴ host cells/well, with the parasite/macrophage ratio remaining at 5:1. The concentration of 4,6-diamidino-2-phenylindole (DAPI) employed for nuclear staining was 4 μM. A PerkinElmer Opera Phenix Plus High Content Imaging System (Waltham, MA) was employed for image collection and analysis, with DAPI excitation at 405 nm and emission at 435-480 nm. For the quantification of the infection, macrophage nuclei were selected first by DAPI staining with area larger than 30 μm². The cytoplasm was selected consequentially based on the nuclei with the digital phase contrast (DPC) channel. Parasites in the cytoplasm were selected based on relative intensity. Parasites per macrophage in each well were exported to a CSV file to generate dose responses, with data analysis performed as described by Joice et al.²⁴

Leishmania CYP51 and CYP5122A1 assays. The cloning, expression, purification, and inhibition assays performed with L. donovani CYP51 and CYP5122A1 were conducted as described in Wang et al.¹⁹

### REFERENCES FOR EXAMPLE 1

1. Jin, H.; McCaffery, J. M.: Grote, E., Ergosterol promotes pheromone signaling and plasma membrane fusion in mating yeast. J. Cell Biol. 2008, 180 (4), 813-826.
2. Shafiei, M.; Peyton, L.; Hashemzadeh, M.; Foroumadi, A., History of the development of antifungal azoles: A review on structures, SAR, and mechanism of action. Bioorg. Chem. 2020, 104, 104240.
3. Monk, B. C.; Tomasiak, T. M.; Keniya, M. V; Huschmann, F. U.; Tyndall, J. D.; O'Connell, J. D., 3rd; Cannon, R. D.; McDonald, J. G.; Rodriguez, A.; Finer-Moore, J. S.; Stroud, R. M., Architecture of a single membrane spanning cytochrome P450 suggests constraints that orient the catalytic domain relative to a bilayer. Proc. Natl. Acad. Sci. USA 2014, 111, 3865-3870.
4. Tyndall, J. D.; Sabherwal, M.; Sagatova, A. A.; Keniya, M. V.; Negroni, J.; Wilson, R. K.; Woods, M. A.; Tietjen, K.; Monk, B. C., Structural and functional elucidation of yeast lanosterol 14α-demethylase in complex with agrochemical antifungals. PLoS One 2016, 11, e0167485.
5. Urbina, J. A.; Payares, G.; Contreras, L. M.; Liendo, A.; Sanoja, C.; Molina, J.; Piras, M.; Piras, R.; Perez, N.; Wincker, P.; Loebenberg, D., Antiproliferative effects and mechanism of action of SCH 56592 against Trypanosoma (Schizotrypanum) cruzi: in vitro and in vivo studies. Antimicrob. Agents Chemother 1998, 42, 1771-1777.
6. Molina, J.; Martins-Filho, O.; Brener, Z.; Romanha, A.; Loebenberg, D.; Urbina, J., Activities of the triazole derivative SCH 56592 (Posaconazole) against drug-resistant strains of the protozoan parasite Trypanosoma (Schizotrypanum) cruzi in immunocompetent and immunosuppressed murine hosts. Antimicrob. Agents Chemother. 2000, 44, 150-155.
7. Molina, I.; Gómez i Prat, J.; Salvador, F.; Treviño, B.; Sulleiro, E.; Serre, N.; Pou, D.; Roure, S.; Cabezos, J.; Valerio, L.; Blanco-Grau, A.; Sánchez-Montalvá, A.; Vidal, X., Pahissa, A., Randomized trial of posaconazole and benznidazole for chronic Chagas' disease. N. Engl. J. Med 2014, 370, 1899-1908.
8. Galvão, E. L.; Rabello, A.; Cota, G. F., Efficacy of azole therapy for tegumentary leishmaniasis: A systematic review and meta-analysis. PLoS One 2017, 12, e0186117.
9. Mashayekhi Goyonlo, V.; Derakhshan, Z.; Darchini-Maragheh, E., Treatment of Cutaneous Leishmaniasis with Allopurinol Plus Itraconazole in Iran. Am. J. Trop. Med. Hyg. 2023, 108, 1164-1166.
10. Sundar, S.; Kumar, K.; Singh, V. P., Ketoconazole in visceral leishmaniasis. Lancet 1990, 336 (8730), 1582-1583.
11. Sundar, S.; Singh, V.; Agrawal, N.; Gibbs, D.; Murray, H., Treatment of kala-azar with oral fluconazole. Lancet 1996, 348, 614.
12. Suryadevara, P.; Olepu, S.; Lockman, J.; Ohkanda, J.; Karimi, M.; Verlinde, C.; Kraus, J.; Schoepe, J.; Van Voorhis, W.; Hamilton, A.; Buckner, F.; Gelb, M., Structurally simple inhibitors of lanosterol 14α-demethylase are efficacious in a rodent model of acute Chagas disease. J. Med Chem. 2009, 52, 3703-3715.
13. Buckner, F. S.; Bahia, M. T.; Suryadevara, P. K.; White, K. L.; Shackleford, D. M.; Chennamaneni, N. K.; Hulverson, M. A.; Laydbak, J. U.; Chatelain, E.; Scandale, I.; Verlinde, C. L.; Charman, S. A.; Lepesheva, G. I.; Gelb, M. H., Pharmacological characterization, structural studies, and in vivo activities of anti-Chagas disease lead compounds derived from tipifarnib. Antimicrob. Agents Chemother. 2012, 56, 4914-4921.
14. Gunatilleke, S.; Calvet, C.; Johnston, J.; Chen, C.; Erenburg, G.; Gut, J.; Engel, J.; Ang, K.; Mulvaney, J.; Chen, S.; Arkin, M.; McKerrow, J.; Podust, L., Diverse inhibitor chemotypes targeting Trypanosoma cruzi CYP51. PLoS Negl. Drop. Dis. 2012, 6, e1736.
15. Lepesheva, G.; Hargrove, T.; Rachakonda, G.; Wawrzak, Z.; Pomel, S.; Cojean, S.; Nde, P.; Nes, W.; Locuson, C.; Calcutt, M.; Waterman, M.; Daniels, J.; Loiseau, P.; Villalta, F., VFV as a new effective CYP51 structure-derived drug candidate for Chagas disease and visceral leishmaniasis. J. Infect. Dis. 2015, 212, 1439-1448.
16. Friggeri, L., Hargrove, T.; Rachakonda, G.; Blobaum, A.; Fisher, P.; de Oliveira, G.; da Silva, C.; Soeiro, M.; Nes, W.; Lindsley, C.; Villalta, F.; Guengerich, F.; Lepesheva, G., Sterol 14α-demethylase structure-based optimization of drug candidates for human infections with the protozoan trypanosomatidae. J. Med. Chem. 2018, 61, 10910-10921.
17. Abdelhameed, A.; Feng, M.; Joice, A. C.; Zywot, E. M.; Jin, Y; La Rosa, C.; Liao, X.; Meeds, H. L.; Kim, Y.; Li, J.; McElroy, C. A.; Wang, M. Z.; Werbovetz, K. A., Synthesis and antileishmanial evaluation of arylimidamide-azole hybrids containing a phenoxyalkyl linker. ACS Infect. Dis. 2021, 7, 1901-1922.
18. Verma, S.; Mehta, A.; Shaha, C., CYP5122A1, a novel cytochrome P450 is essential for survival of Leishmania donovani. PLoS One 2011, 6, e25273.
19. Wang, M.; Jin, Y.; Basu, S.; Feng, M.; Ning, Y.; Munasinghe, I.; Joachim, A.; Li, J.; Madden, R.; Burks, H.; Gao, P.; Perera, C.; Werbovetz, K.; Zhang, K., CYP5122A1 encodes an essential sterol C4-methyl oxidase in Leishmania donovani and determines the antileishmanial activity of antifungal azoles. Res. Sq. 2023, doi: 10.21203/rs.3.rs-3185204/v1.
20. Bard, M.; Bruner, D. A.; Pierson, C. A.; Lees, N. D.; Biermann, B.; Frye, L.; Koegel, C.; Barbuch, R., Cloning and characterization of ERG25, the Saccharomyces cerevisiae gene encoding C4 sterol methyl oxidase. Proc. Natl. Acad. Sci. USA 1996, 93, 186-190.
21. Lee, A. K.; Banta, A. B.; Wei, J. H.; Kiemle, D. J.; Feng, J.; Giner, J. L.; Welander, P. V., C4 sterol demethylation enzymes distinguish bacterial and eukaryotic sterol synthesis. Proc. Natl. Acad. Sci. USA 2018, 115, 5884-5889.
22. Knaggs, S.; Malkin, H.; Osborn, H. M.; Williams, N. A.; Yaqoob, P., New prodrugs derived from 6-aminodopamine and 4-aminophenol as candidates for melanocyte-directed enzyme prodrug therapy (MDEPT). Org. Biomol. Chem. 2005, 3, 4002-4010.
23. Siqueira-Neto, J.; Moon, S.; Jang, J.; Yang, G.; Lee, C.; Moon, H.; Chatelain, E.; Genovesio, A.; Cechetto, J.; Freitas-Junior, L., An image-based high-content screening assay for compounds targeting intracellular Leishmania donovani amastigotes in human macrophages. PLoS Negl. Trop. Dis. 2012, 6, e1671.
24. Joice, A.; Yang, S.; Farahat, A.; Meeds, H., Feng, M.; Li, J.; Boykin, D.; Wang, M.; Werbovetz, K., Antileishmanial efficacy and pharmacokinetics of DB766-azole combinations. Antimicrob. Agents Chemother. 2018, 62, e01129-17.
25. Gottlieb, H.; Kotlyar, V.; Nudelman, A., NMR chemical shifts of common laboratory solvents as trace impurities. J. Org. Chem. 1997, 62, 7512-7515.
26. Feng, M.; Jin, Y.; Yang, S.; Joachim, A.; Ning, Y.; Mori-Quiroz, L.; Fromm, J.; Perera, C.; Zhang, K.; Werbovetz, K.; Wang, M. Z., Sterol profiling of Leishmania parasites using a new HPLC-tandem mass spectrometry-based method and antifungal azoles as chemical probes reveals a key intermediate sterol that supports a branched ergosterol biosynthetic pathway. Int. J. Parasitol. Drugs Drug Resist. 2022, 20, 27-42.
27. Zhu, X.; Van Horn, K.; Barber, M.; Yang, S.; Wang, M. Z.; Manetsch, R.; Werbovetz, K., SAR refinement of antileishmanial N²,N⁴-disubstituted quinazoline-2,4-diamines. Bioorg. Med. Chem. 2015, 23, 5182-5189.
28. Abdelhameed, A.; Liao, X.; McElroy, C.; Joice, A.; Rakotondraibe, L.; Li, J.; Slebodnick, C.; Guo, P.; Wilson, W.; Werbovetz, K., Synthesis and antileishmanial evaluation of thiazole orange analogs. Bioorg. Med. Chem. Lett. 2020, 30, 126725.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

1. A compound of Formula I 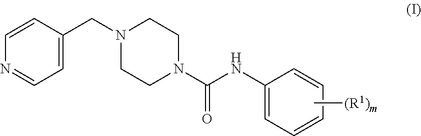
or a pharmaceutically acceptable salt or derivative thereof;
wherein:
R¹ is selected from halo, cyano, azido, C₁-C₆ alkyl, C₁-C₆ haloalkyl, C₂-C₆ alkenyl, C₂-C₆ alkynyl, (C₃-C₆ cycloalkyl)-(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;
m is 0, 1, 2, 3, 4, or 5;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol;
with the proviso that 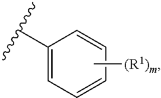
cannot be 3,5-dimethylphenyl.

2. The compound of claim 1, wherein 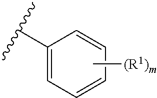
is selected from:

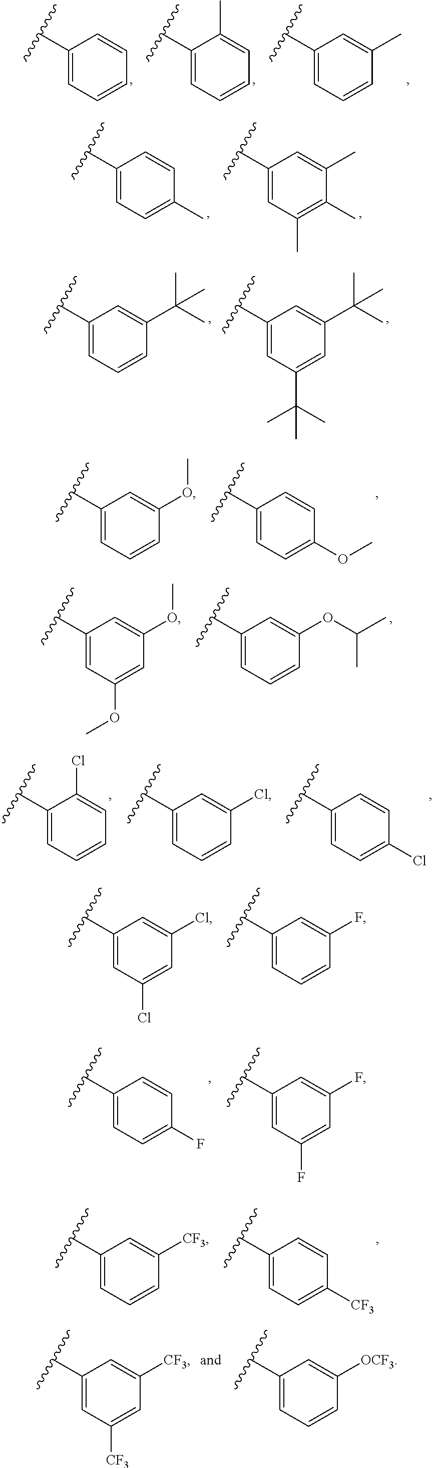

3. A compound of claim 1 selected from 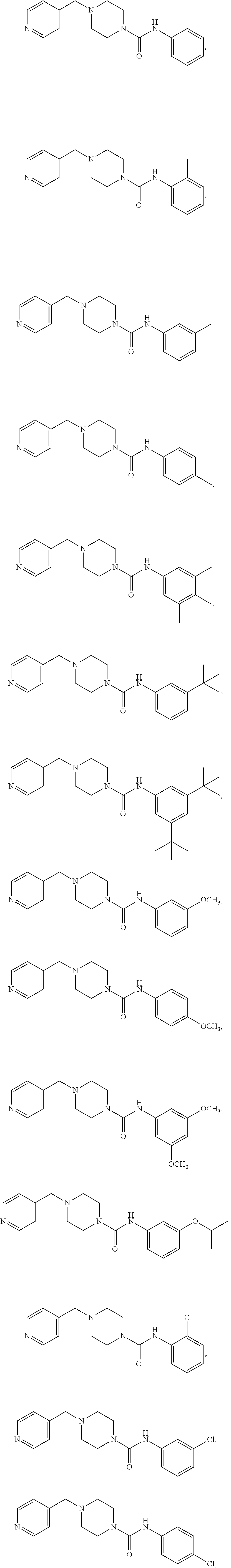 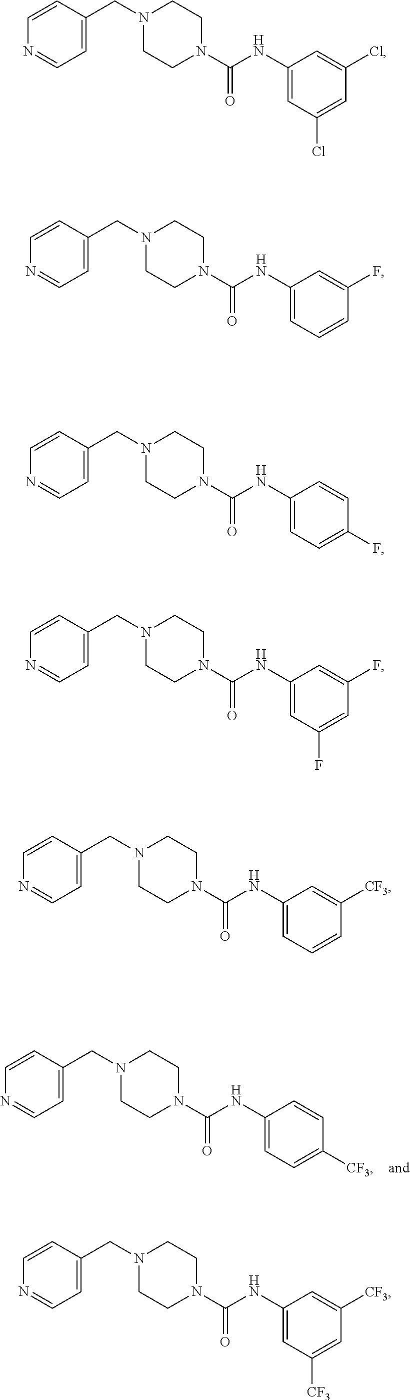
or a pharmaceutically acceptable salt or derivative thereof.

4. A compound of Formula II 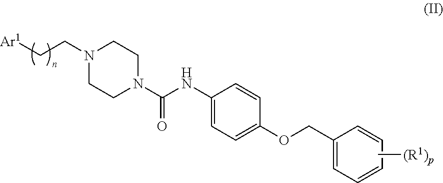
or a pharmaceutically acceptable salt thereof,
wherein:
R¹ is selected halo, cyano, azido, C₁-C₆ alkyl, C₁-C₆ haloalkyl, C₂-C₆ alkenyl, C₂-C₆ alkynyl, (C₃-C₆ cycloalkyl)-(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N)S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{x}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;
n is 0, 1, or 2;
p is 1, 2, 3, 4, or 5;
Ar¹ is 6- to 10-membered monocyclic or bicyclic aryl or 5- to 10-membered monocyclic or bicyclic heteroaryl, wherein Ar¹ is optionally substituted with one or more Z groups as allowed by valency;
Z is selected from hydrogen, halo, cyano, azido, C₁-C₆ alkyl, C₁-C₆ haloalkyl, C₂-C₆ alkenyl, C₂-C₆ alkynyl, (C₃-C₆ cycloalkyl)-(C₀-C₃ alkyl)-, (3- to 8-membered monocyclic or bicyclic heterocycle)-(C₀-C₃ alkyl)-, (6- to 10-membered monocyclic or bicyclic aryl)-(C₀-C₃ alkyl)-, (5- to 10-membered monocyclic or bicyclic heteroaryl)-(C₀-C₃ alkyl)-, R^{x}O—(C₀-C₃ alkyl)-, R^{x}S—(C₀-C₃ alkyl)-, (R^{x}R^{y}N)—(C₀-C₃ alkyl)-, R^{x}O—C(O)—(C₀-C₃ alkyl)-, R^{x}S—C(O)—(C₀-C₃ alkyl)-, (R^{x}R^{y}N) C(O)—(C₀-C₃ alkyl)-, R^{x}O—S(O)₂—(C₀-C₃ alkyl)-, (R^{x}R^{y}N) S(O)₂—(C₀-C₃ alkyl)-, R^{z}C(O)—O—(C₀-C₃ alkyl)-, R^{z}C(O)—(R^{x}N)—(C₀-C₃ alkyl)-, R^{z}S(O)₂—O—(C₀-C₃ alkyl)-, R^{z}S(O)₂—(R^{z}N)—(C₀-C₃ alkyl)-, R^{z}C(O)—(C₀-C₆ alkyl)-, R^{z}S(O)—(C₀-C₃ alkyl)-, and R^{z}S(O)₂—(C₀-C₃ alkyl)-;
R^{x} and R^{y} are independently selected at each occurrence from hydrogen, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, and (5- to 10-membered monocyclic or bicyclic heteroaryl)-(C₀-C₃ alkyl)-, each of which may be optionally substituted with one or more Y groups as allowed by valency;
R^{z} is independently selected at each occurrence from hydrogen, halo, C₁-C₆alkyl, C₁-C₆haloalkyl, C₂-C₆alkenyl, C₂-C₆alkynyl, (C₃-C₇cycloalkyl)-(C₀-C₃ alkyl)-, (4- to 6-membered heterocycle)-(C₀-C₃ alkyl)-, (5- to 10-membered monocyclic or bicyclic aryl)-(C₀-C₃ alkyl)-, (5- to 10-membered monocyclic or bicyclic heteroaryl)-(C₀-C₃ alkyl)-, —OR^{x}, —SR^{x}, and —NR^{x}R^{y}, each of which may be optionally substituted with one or more Y groups as allowed by valency; and
Y is independently selected at each occurrence from alkyl, haloalkyl, alkoxy, alkenyl, alkynyl, aryl, heteroaryl, cycloalkyl, heterocycle, aldehyde, amino, carboxylic acid, ester, ether, halo, hydroxy, keto, nitro, cyano, azido, oxo, silyl, sulfo-oxo, sulfonyl, sulfone, sulfoxide, sulfonylamino, or thiol.

5. The compound of claim 4, wherein Ar¹ is selected from:

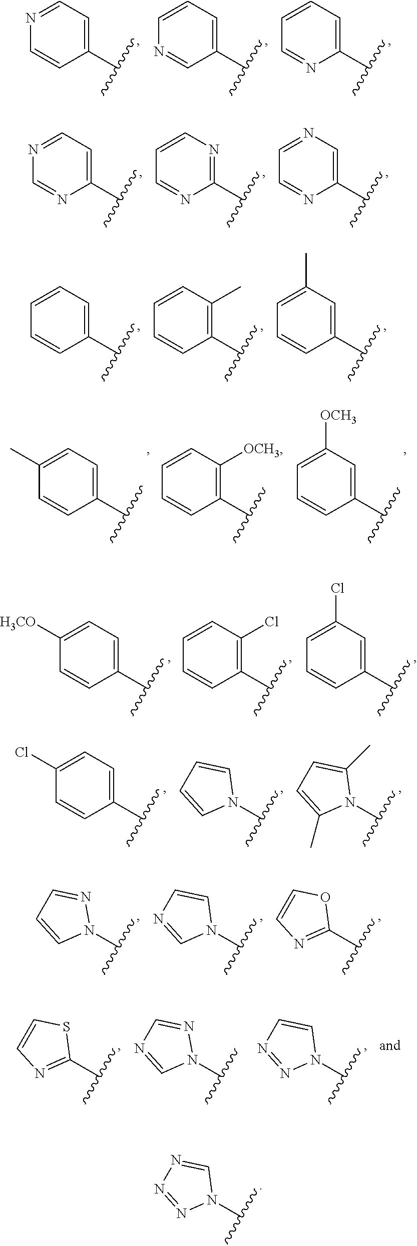

6. The compound of claim 4, wherein n is 0.

7. The compound of claim 4, wherein n is 1.

8. The compound of claim 4, wherein n is 2.

9. The compound of claim 3, wherein 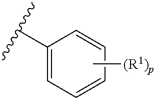
is selected from:

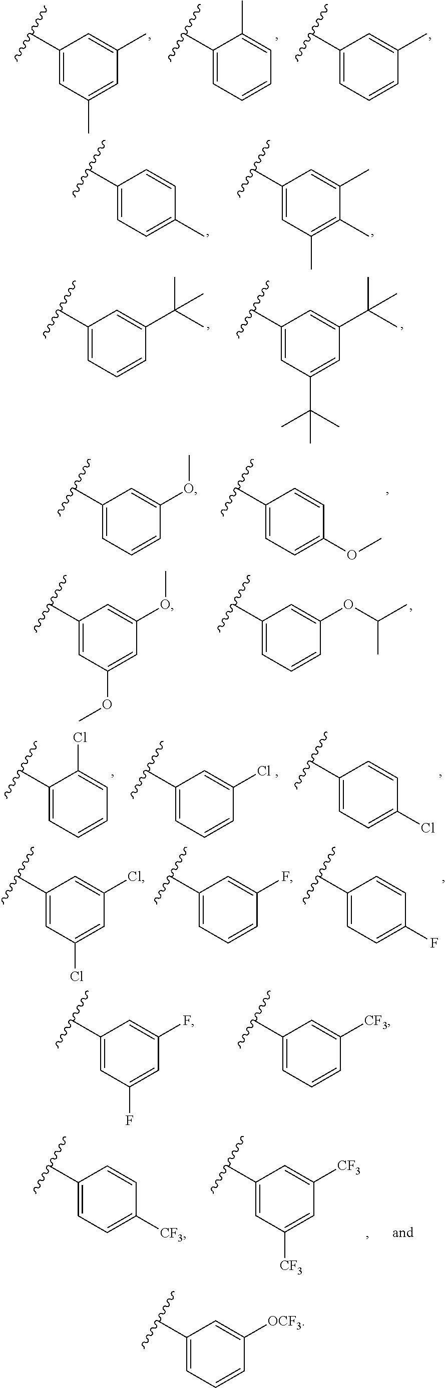

10. A compound selected from: 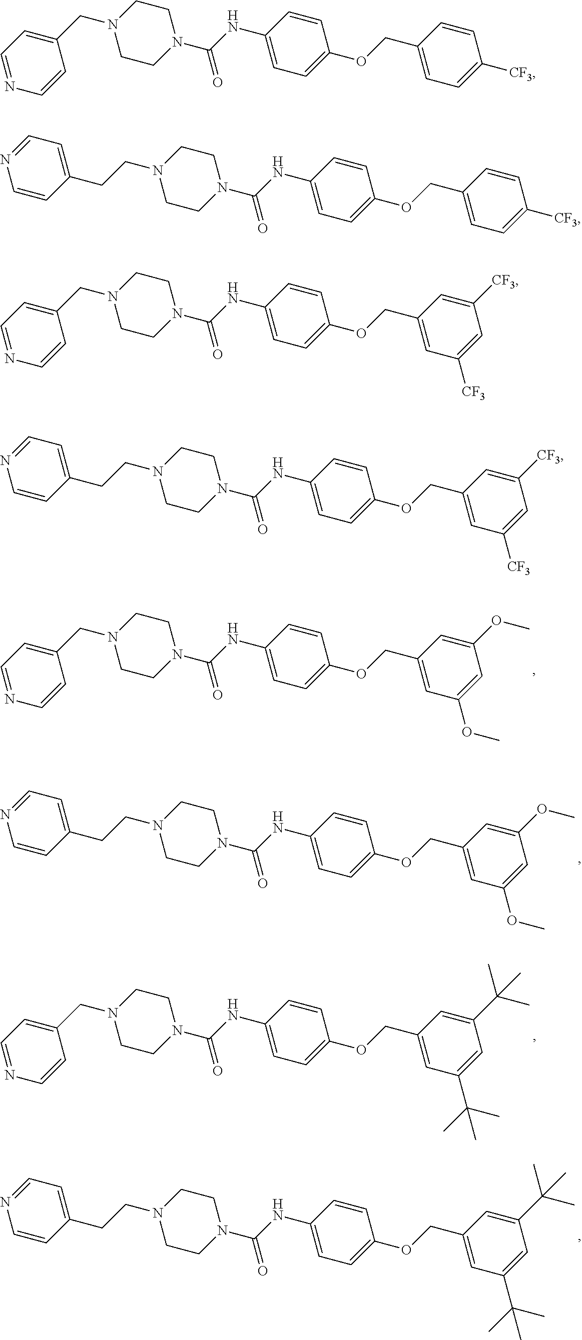 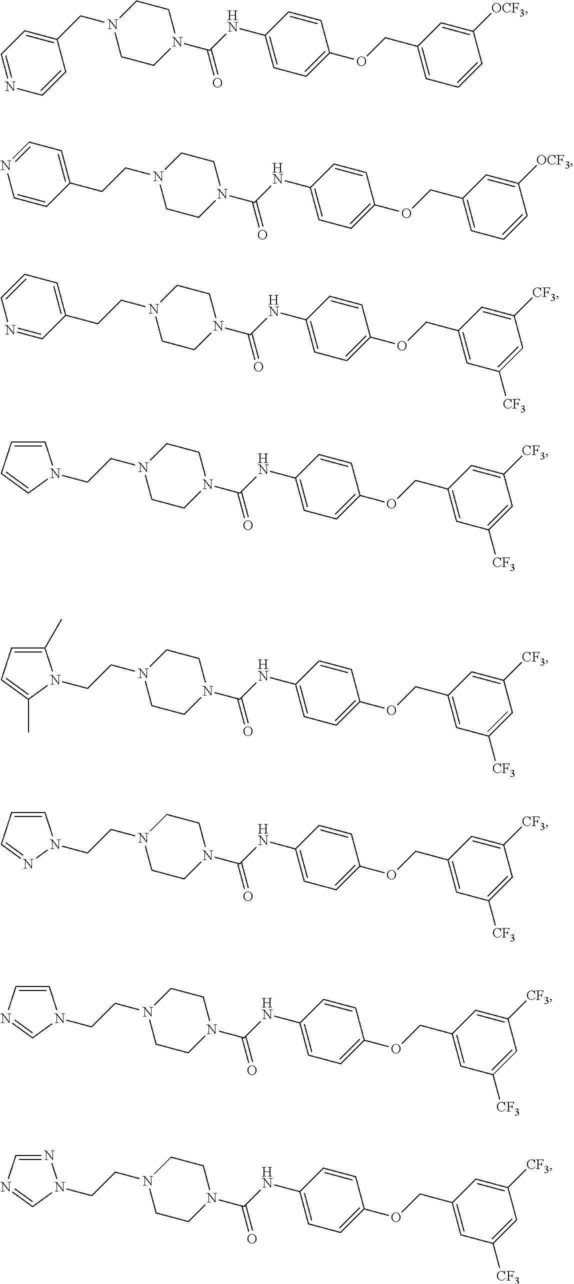 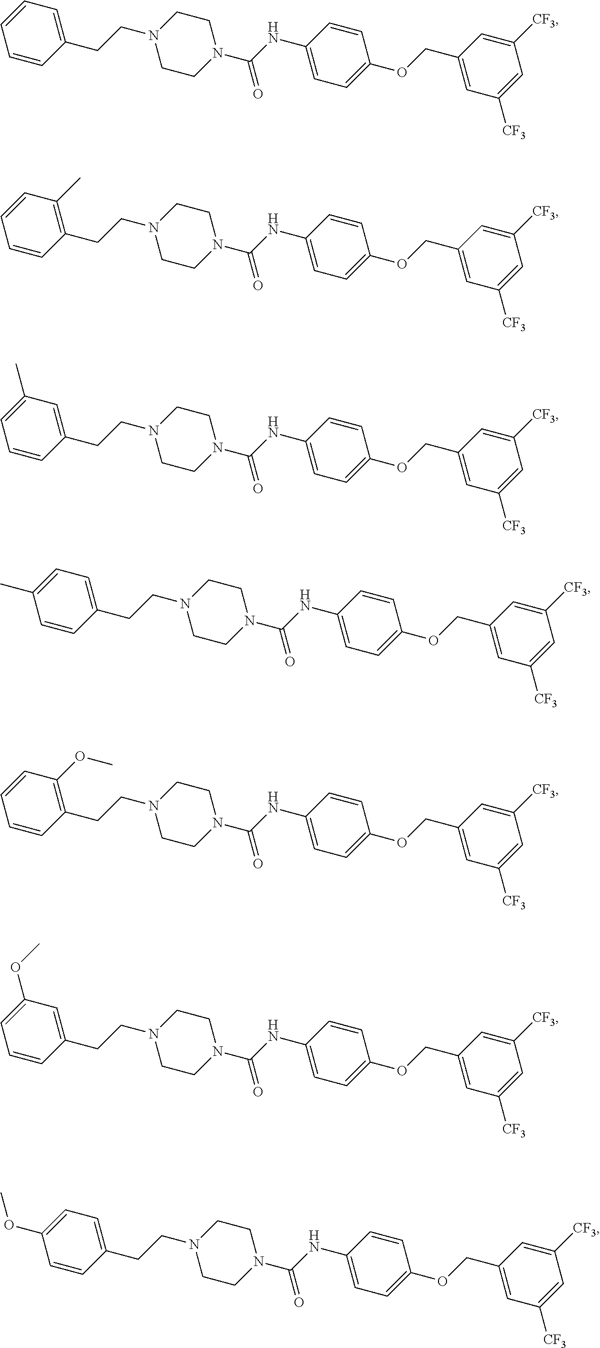 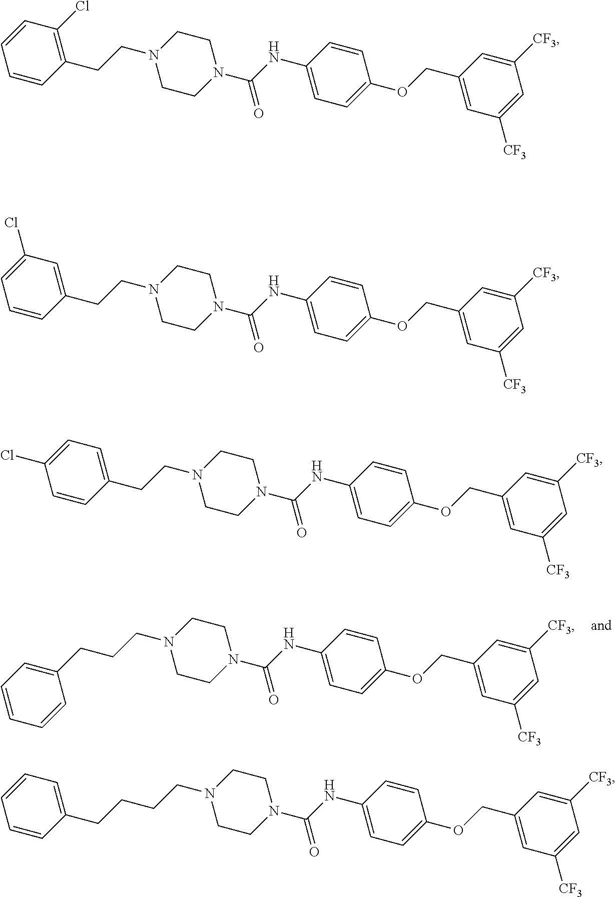 [SMILES: O=C(Nc1ccc(OCc2cc(C(F)(F)F)cc(C(F)(F)F)c2)cc1)N1CCN(CCc2ccccc2Cl)CC1 ; O=C(Nc1ccc(OCc2cc(C(F)(F)F)cc(C(F)(F)F)c2)cc1)N1CCN(CCc2cccc(Cl)c2)CC1 ; O=C(Nc1ccc(OCc2cc(C(F)(F)F)cc(C(F)(F)F)c2)cc1)N1CCN(CCc2ccc(Cl)cc2)CC1 ; O=C(Nc1ccc(OCc2cc(C(F)(F)F)cc(C(F)(F)F)c2)cc1)N1CCN(CCCc2ccccc2)CC1 ; O=C(Nc1ccc(OCc2cc(C(F)(F)F)cc(C(F)(F)F)c2)cc1)N1CCN(CCCCc2ccccc2)CC1]
or a pharmaceutically acceptable salt or derivative thereof.

11. A pharmaceutical composition comprising a compound of claim 1, or a pharmaceutically acceptable salt or derivative thereof, and a pharmaceutically acceptable carrier or excipient.

12. A method of treating or preventing an infection with a parasitic protozoa in a subject in need thereof comprising administering to the subject a therapeutically effective amount of a compound of claim 1, or a pharmaceutically acceptable salt or derivative thereof.

13. The method of claim 12, wherein the parasitic protozoa expresses CYP51 and/or CYP5122A1.

14. The method of claim 12, wherein the parasitic protozoa comprises a Leishmania parasite.

15. The method of claim 14, wherein the Leishmania parasite comprises L. aethiopica, L. amazonensis, L. arabica, L. archibaldi, L. aristedesi, L. viannia, L. braziliensis, L. chagasi, L. colombiensis, L. deanei, L. donovani, L. enriettii, L. equatorensis, L. forattinii, L. garnhami, L. gerbili, L. guyanensis, L. herreri, L. hertigi, L. infantum, L. killicki, L. lainsoni, L. major, L. mexicana, L. naiffi, L. panamensis, L. peruviana, L. pifanoi, L. shawi, L. tarentolae, L. tropica, L. turanica, or L. venezuelensis.

16. The method of claim 12, wherein the infection comprises cutaneous leishmaniasis, mucocutaneous leishmaniasis, or visceral leishmaniasis.

17. The method of claim 12, wherein the parasitic protozoa comprises a Trypanosoma parasite.

18. The method of claim 17, wherein the Trypanosoma parasite comprises T. ambystomae, T. avium, T. boissoni, T. brucei, T. cruzi, T. congolense, T. equinum, T. equiperdum, T. evansi, T. everetti, T. hosei, T. irwini, T. lewisi, T. melophagium, T. paddae, T. parroti, T. percae, T. rangeli, T. rotatorium, T. rugosae, T. sergenti, T. simiae, T. sinipercae, T. suis, T. theileri, T. triglae, or T. vivax.

19. The method of claim 12, wherein the infection comprises African trypanosomiasis, Chagas disease, nagana, and surra.

20. The method of claim 12, wherein the subject is a human, dog, cat, cow, horse, sheep, pig, bird, amphibian, or fish.
